(19) United States
(12) Patent Application Publication
(10) Pub. No.: US 2022/0102643 A1
(43) Pub. Date: Mar. 31, 2022

(54) COMPOUND FOR ORGANIC ELECTRONIC ELEMENT, ORGANIC ELECTRONIC ELEMENT USING THE SAME, AND AN ELECTRONIC DEVICE THEREOF

(71) Applicant: DUK SAN NEOLUX CO., LTD., Cheonan-si (KR)

(72) Inventors: Dong Hee SHIN, Cheonan-si (KR); Hyung Dong LEE, Cheonan-si (KR); Sun Hee LEE, Cheonan-si (KR); Dae Sic KIM, Cheonan-si (KR); Soung Yun MUN, Cheonan-si (KR); Young Hoon KANG, Cheonan-si (KR)

(73) Assignee: DUK SAN NEOLUX CO., LTD., Cheonan-si (KR)

(21) Appl. No.: 17/305,850

(22) Filed: Jul. 15, 2021

(30) Foreign Application Priority Data
Jul. 16, 2020 (KR) 10-2020-0087951

(51) Int. Cl. H01L 51/00; C07D 498/08; C09K 11/06; C07D 413/04; C07D 413/10; C07D 247/00

(52) U.S. Cl. CPC H01L 51/0061; C07D 498/08; C09K 11/06; H01L 51/5016; C07D 413/10; C07D 247/00; C07D 413/04

(57) ABSTRACT

Provides are a compound capable of improving the luminous efficiency, stability and lifespan of a device, an organic electronic element employing the same, and an electronic device thereof.

### BACKGROUND

### Technical Field

The present invention relates to a compound for an organic electronic element, an organic electronic element using the same, and an electronic device thereof.

### Background Art

In general, organic light emitting phenomenon refers to a phenomenon that converts electric energy into light energy by using an organic material. An organic electronic element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer and the like.

A material used as an organic material layer in an organic electronic element may be classified into a light emitting material and a charge transport material, such as a hole injection material, a hole transport material, an electron transport material, an electron injection material and the like depending on its function.

Lifespan and efficiency are the most problematic in organic electroluminescent device, and as displays become larger, these problems of efficiency and lifespan must be solved. Efficiency, lifespan, and driving voltage are related to each other, and when the efficiency is increased, the driving voltage is relatively decreased, and as the driving voltage is decreased, crystallization of the organic material due to Joule heating generated during driving decreases, and as a result, the lifespan tends to increase.

However, the efficiency cannot be maximized simply by improving the organic material layer. This is because, when the energy level and T1 value between each organic material layer, and the intrinsic properties of the material (mobility, interfacial properties, etc.) are optimally combined, a long lifespan and high efficiency can be achieved at the same time.

Also, in order to solve the problem of light emission in the hole transport layer in recent organic electroluminescent devices, an emitting-auxiliary layer must exist between the hole transport layer and the emitting layer, and it is time to develop different emitting-auxiliary layers according to each emitting layer (R, G, B).

In general, electrons are transferred from the electron transport layer to the emitting layer, and holes are transferred from the hole transport layer to the emitting layer, and excitons are generated by recombination.

However, since the material used for the hole transport layer should have a low HOMO value, most have a low T1 value. As a result, excitons generated in the emitting layer are transferred to the hole transport layer, resulting in charge unbalance in the emitting layer to emit light at the hole transport layer interface.

When light is emitted at the hole transport layer interface, the color purity and efficiency of the organic electronic element are lowered, and the lifespan is shortened. Therefore, it is urgently required to develop an emitting-auxiliary layer having a high T1 value and having a HOMO level between the HOMO energy level of the hole transport layer and the HOMO energy level of the emitting layer.

Furthermore, it is necessary to develop a hole injection layer material that delays the penetration and diffusion of metal oxides from the anode electrode (ITO) into the organic layer, which is one of the causes of shortening the lifespan of organic electronic element, and that has stable characteristics, that is, a high glass transition temperature, even against Joule heating generated during device driving. The low glass transition temperature of the hole transport layer material has a characteristic of lowering the uniformity of the thin film surface during device driving, which is reported to have a significant effect on device lifespan. Moreover, OLED devices are mainly formed by a deposition method, and it is necessary to develop a material that can withstand a long time during deposition, that is, a material with strong heat resistance.

That is, in order to sufficiently exhibit the excellent characteristics of the organic electronic element, it should be preceded that materials constituting the organic material layer in the element, such as a hole injection material, a hole transport material, an emitting material, an electron transport material, an electron injection material, an emitting-auxiliary layer material, etc. are supported by a stable and efficient material, but the development of a stable and efficient organic material layer material for an organic electronic element has not yet been sufficiently made.

Accordingly, the development of new materials continues to be demanded.

### DETAILED DESCRIPTION OF THE INVENTION

### Summary

In order to solve the problems of the background art described above, the present invention has revealed a compound having a novel structure, and also found that when this compound is applied to an organic electronic element, the luminous efficiency, stability and lifespan of the element can be greatly improved.

Accordingly, an object of the present invention is to provide a novel compound, an organic electronic element using the same, and an electronic device thereof.

### Technical Solution

The present invention provides a compound represented by Formula 1.

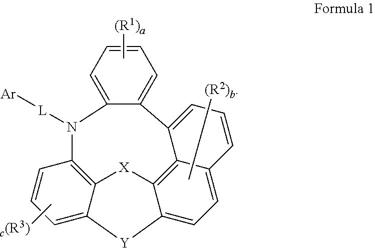

In another aspect, the present invention provides an organic electronic element comprising the compound represented by Formula 1 and an electronic device thereof.

### Effects of the Invention

By using the compound according to the present invention, high luminous efficiency, low driving voltage and high heat resistance of the element can be achieved, and color purity and lifespan of the element can be greatly improved.

### BRIEF DESCRIPTION OF THE DRAWINGS

FIG. 1 to FIG. 3 are each an exemplary view of an organic electroluminescent device according to the present invention.

FIG. 4 shows a Formula according to an aspect of the present invention.

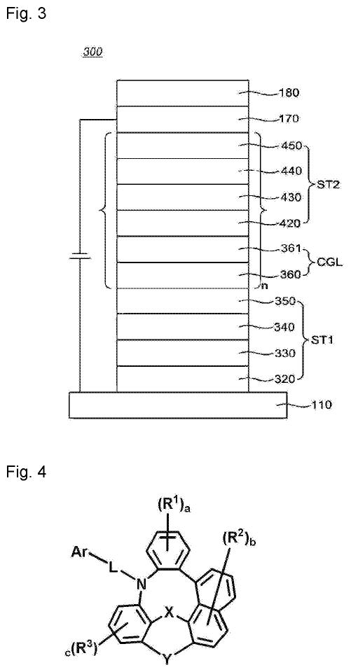

### DETAILED DESCRIPTION

Hereinafter, some embodiments of the present invention will be described in detail.

Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected coupled” or “connected” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

The terms “aryl group” and “arylene group” used in the present invention have 6 to 60 carbon atoms, respectively, unless otherwise specified, but are not limited thereto. In the present invention, an aryl group or an arylene group means a single ring or multiple ring aromatic, and includes an aromatic ring formed by an adjacent substituent joining or participating in a reaction.

For example, the aryl group may be a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, but is not limited thereto, has 2 to 60 carbon atoms, includes any one of a single ring or multiple ring, and may include heteroaliphadic ring and heteroaromatic ring. Also, the heterocyclic group may also be formed in conjunction with an adjacent group.

Unless otherwise stated, the term “heteroatom”, as used herein, represents at least one of N, O, S, P, or Si.

Also, the term “heterocyclic group” may include a ring including SO₂ instead of carbon consisting of cycle. For example, “heterocyclic group” includes the following compound.

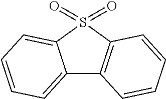 [SMILES: O=S1(=O)c2ccccc2-c2ccccc21]

Unless otherwise stated, the term “fluorenyl group” or “fluorenylene group”, as used herein, means a monovalent or divalent functional group, in which R, R′ and R″ are all hydrogen in the following structures, and the term “substituted fluorenyl group” or “substituted fluorenylene group” means that at least one of the substituents R, R′, R″ is a substituent other than hydrogen, and include those in which R and R′ are bonded to each other to form a spiro compound together with the carbon to which they are bonded.

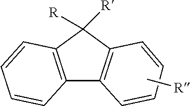

The term “spiro compound”, as used herein, has a ‘spiro union’, and a spiro union means a connection in which two rings share only one atom. At this time, atoms shared in the two rings are called ‘spiro atoms’, and these compounds are called ‘monospiro-’, ‘di-spiro-’ and ‘tri-spiro-’, respectively, depending on the number of spiro atoms in a compound.

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds include, but are not limited thereto, one or more heteroatoms.

Also, unless expressly stated, as used herein, “substituted” in the term “substituted or unsubstituted” means substituted with one or more substituents selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited to these substituents.

Also, unless there is an explicit explanation, the formula used in the present invention is the same as the definition of the substituent by the exponent definition of the following formula.

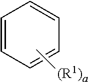

here, when a is an integer of zero, the substituent R¹ is absent, when a is an integer of 1, the sole substituent R¹ is linked to any one of the carbon constituting the benzene ring, when a is an integer of 2 or 3, each is combined as follows, where R¹ may be the same or different from each other, when a is an integer of 4 to 6, it is bonded to the carbon of the benzene ring in a similar manner, while the indication of the hydrogen bonded to the carbon forming the benzene ring is omitted.

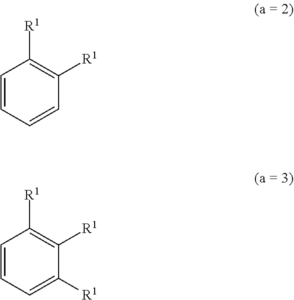

As used herein, the term “absence” means no binding unless otherwise specified.

Hereinafter, a compound according to an aspect of the present invention and an organic electronic device including the same will be described.

The present invention provides a compound represented by Formula 1.

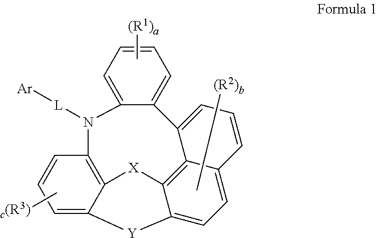

wherein each symbol may be defined as follows.

1) X and Y are independently of each other are O, S, NR′, CR′R″ or absent;

2) Wherein R′ and R″ are independently of each other hydrogen; deuterium; halogen; C₁-C₆ alkyl group; a C₂-C₆₀ alkenyl group; a C₂-C₆₀ alkynyl group; a C₁-C₆₀ alkoxy group; a C₆-C₆₀ aryloxy group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; or R′ and R″ may be bonded to each other to form a spiro.

When R′ and R″ are an alkyl group, it may be preferably a C₁-C₃₀ alkyl group, and more preferably a C₁-C₂₄ alkyl group.

When R′ and R″ are an alkenyl group, it may be preferably a C₂-C₃₀ alkenyl group, and more preferably a C₂-C₂₄ alkenyl group.

When R′ and R″ are an alkynyl group, it may be preferably a C₂-C₃₀ alkynyl group, and more preferably a C₂-C₂₄ alkynyl group.

When R′ and R″ are an alkoxyl group, it may be preferably a C₁-C₃₀ alkoxyl group, and more preferably a C₁-C₂₄ alkoxyl group.

When R′ and R″ are an aryloxy group, it may be preferably a C₆-C₃₀ aryloxy group, and more preferably a C₆-C₂₄ aryloxy group.

When R′ and R″ are an aryl group, it may be preferably a C₆-C₃₀ aryl group, and more preferably a C₆-C₂₄ aryl group, for example, it may be phenylene, biphenyl, naphthalene, terphenyl, etc.

When R′ and R″ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazole, dibenzofuran, benzothienopyrimidine, benzofuropyrimidine, phenothiazine, phenylphenothiazine, etc.

When R′ and R″ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

3) R¹, R² and R³ are each the same or different, and each independently selected from a group consisting of hydrogen; deuterium; halogen; cyano group; nitro group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₆₀ alkyl group; a C₂-C₆₀ alkenyl group; a C₂-C₆₀ alkynyl group; a C₁-C₆₀ alkoxy group; a C₆-C₆₀ aryloxy group; and -L′-N(R^{a})(R^{b}); or a plurality of adjacent R¹s, or a plurality of R²s, or a plurality of R³s may be bonded to each other to form a ring.

When R¹, R² and R³ are an aryl group, they may be preferably a C₆-C₃₀ aryl group, most preferably a C₆-C₂₅ aryl group, exemplarily, they may be phenyl, biphenyl, naphthyl, phenanthrene, terphenyl, and the like.

When R¹, R² and R³ are a heterocyclic group, they may be preferably a C₂-C₃₀ heterocyclic group, and more preferably a C₂-C₂₄ heterocyclic group, for example, they may be pyrazine, thiophene, pyridine, pyrimidoindole, 5-phenyl-5H-pyrimido[5,4-b]indole, quinazoline, benzoquinazoline, carbazole, dibenzoquinazole, dibenzofuran, benzothienopyrimidine, benzofuropyrimidine, phenothiazine, phenylphenothiazine, etc.

When R¹, R² and R³ are a fused ring group, they may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

When R¹, R² and R³ are an alkyl group, they may be preferably an C₁-C₃₀ alkyl group, more preferably an C₁-C₂₄ alkyl group.

When R¹, R² and R³ are an alkenyl group, they may be preferably an C₂-C₃₀ alkenyl group, more preferably an C₂-C₂₄ alkenyl group.

When R¹, R² and R³ are an alkynyl group, they may be preferably a C₂-C₃₀ alkynyl group, and more preferably a C₂-C₂₄ alkynyl group.

When R¹, R² and R³ are an alkoxy group, they may be preferably a C₁-C₃₀ alkoxy group, and more preferably a C₁-C₂₄ alkoxy group.

When R¹, R² and R³ are an aryloxy group, they may be preferably a C₆-C₃₀ aryloxy group, and more preferably a C₆-C₂₄ aryloxy group.

4) wherein L and L′ are each independently selected from the group consisting of single bond; a C₆-C₆₀ arylene group; a fluorenylene group; a C₂-C₆₀ heterocyclic group including at least one heteroatom of O, N, S, Si or P; and a C₃-C₆₀ aliphatic ring;

wherein L and L′ are an arylene group, it may be preferably a C₆-C₃₀ arylene group, more preferably a C₆-C₂₄ arylene group, for example, phenylene, biphenyl, naphthalene, terphenyl, etc.
wherein L and L′ are a heterocyclic group, it may be preferably a C₂-C₃₀ heterocyclic group, and more preferably a C₂-C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazole, dibenzofuran, benzothienopyrimidine, benzofuropyrimidine, phenothiazine, phenylphenothiazine, etc.
wherein L and L′ are an aliphatic ring group, it may be preferably an C₃-30 aliphatic ring, and more preferably an C₃-C₂₄ aliphatic ring,

5) When Ar, R^{a} and R^{b} are each independently a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring;

When Ar, R^{a} and R^{b} are an aryl group, it may be preferably a C₆-C₃₀ aryl group, and more preferably a C₆-C₂₅ aryl group, for example, phenyl, biphenyl, naphthyl, phenanthrene, terphenyl, etc.

When Ar, R^{a} and R^{b} are a heterocyclic group, it may be preferably a C₂-C₃₀ heterocyclic group, and more preferably a C₂-C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazole, dibenzofuran, benzothienopyrimidine, benzofuropyrimidine, phenothiazine, phenylphenothiazine, etc.

When Ar, R^{a} and R^{b} are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

6) a is an integer from 0 to 4, b is an integer from 0 to 5, and c is an integer from 0 to 3.

7) wherein, the aryl group, arylene group, heterocyclic group, fluorenyl group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxy group; C₁-C₂₀alkyl group; C₂-C₂₀alkenyl group; C₂-C₂₀alkynyl group; C₆-C₂₀ aryl group; C₆-C₂₀ aryl group substituted with deuterium; a fluorenyl group; C₂˜C₂₀ heterocyclic group; C₃-C₂₀ cycloalkyl group; C₇-C₂₀ arylalkyl group; C₈-C₂₀ arylalkenyl group; and -L′-NR^{a}R^{b}; and the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, the compound represented by Formula 1 is represented by any one of Formulas 2 to 4

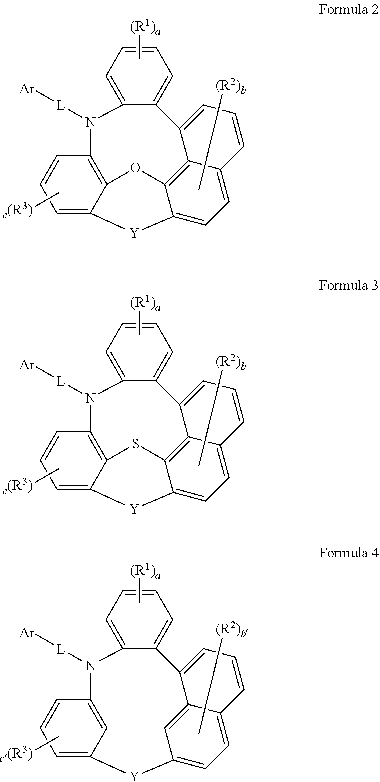

### {Wherein,

1) Y, L, Ar, R¹, R², R³, a, b and c are the same as defined in Formula 1,

2) b′ is an integer from 0 to 6, and c′ is an integer from 0 to 4.}Also, the compound represented by Formula 1 is represented by any one of Formulas 5 to 9:

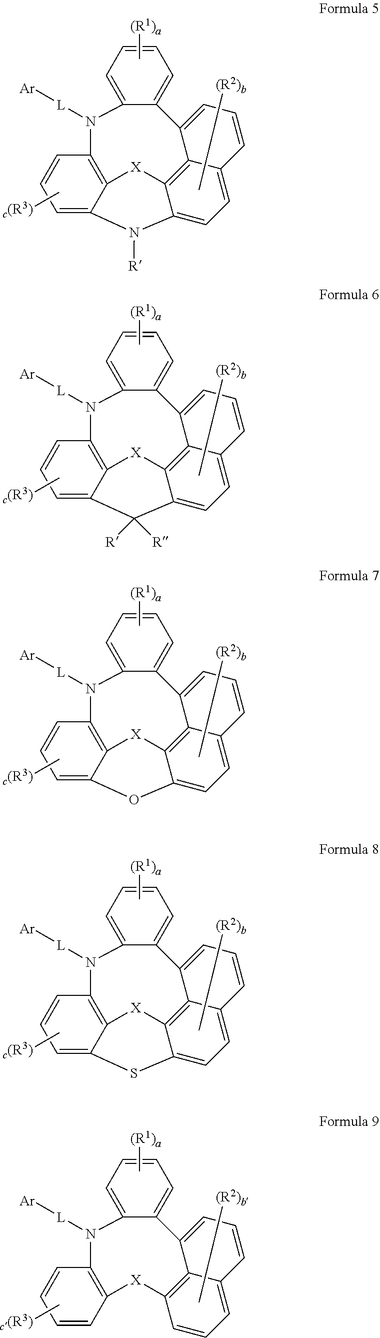

{wherein,

1) X, L, Ar, R¹, R², R³, R′, R″, a, b and c are the same as defined in Formula 1}

2) b′ and c′ are the same as defined in Formula 2}

Also, Ar of Formula 1 is represented by Formula (A)

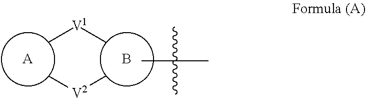

{wherein,

1) A and B are each independently a C₆-C₂₀ aryl group; or C₄-C₂₀ heterocyclic group; and

2) V¹ and V² are each independently a single bond, O, S, NR′ or CR′R″, except that both V¹ and V² are single bonds;

3) R and R″ are as defined in Formula 1 above,

4) [symbol] indicates the binding position.}

Also, at least one of R¹ to R³ in Formula 1 is represented by Formula (B) or Formula (C)

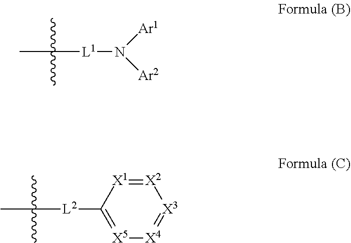

In Formula (B) or Formula (C), each symbol may be defined as follows.

1) L¹ and L² are the same as the definition of L in Formula 1,

2) Ar¹ and Ar² are the same as the definition of Ar in Formula 1,

3) X¹, X², X³, X⁴ and X⁵ are independently of each other CRx or N, provided that at least one of X¹, X², X³, X⁴ and X⁵ is N,

4) wherein Rx is hydrogen; deuterium; halogen; cyano group; nitro group; a C₆-C₆₀ aryl group; or a C₂-C₆₀ heterocyclic group including at least one hetero atom of O, N, S, Si or P; a fluorenyl group; a fused ring group of a C₃-C₆₀ aliphatic ring and a C₆-C₆₀ aromatic ring; C₁-C₆₀ alkyl group; a C₂-C₆₀ alkenyl group; a C₂-C₆₀ alkynyl group; a C₁-C₀₀ alkoxyl group; a C₆-C₆₀ aryloxy group; and a plurality of adjacent Rx may be bonded to each other to form a ring.

When Rx is an aryl group, it may be preferably a C₆-C₃₀ aryl group, and more preferably a C₆-C₂₄ aryl group, for example, phenyl, biphenyl, naphthyl, phenanthrene, terphenyl, etc.

When Rx is a heterocyclic group, it may be preferably a C₂-C₃₀ heterocyclic group, and more preferably a C₂-C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazole, dibenzofuran, benzothienopyrimidine, benzofuropyrimidine, phenothiazine, phenylphenothiazine, etc.

When Rx is a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

When Rx is an alkyl group, it may be preferably an C₁˜C₃₀ alkyl group, more preferably an C₁˜C₂₄ alkyl group.

When Rx is an alkenyl group, it may be preferably an C₂˜C₃₀ alkenyl group, more preferably an C₂˜C₂₄ alkenyl group.

When Rx is an alkynyl group, it may be preferably a C₂-C₃₀ alkynyl group, and more preferably a C₂-C₂₄ alkynyl group.

When Rx is an alkoxyl group, it may be preferably a C₁-C₃₀ alkoxyl group, and more preferably a C₁-C₂₄ alkoxyl group.

When Rx is an aryloxy group, it may be preferably a C₆-C₃₀ aryloxy group, and more preferably a C₆-C₂₄ aryloxy group.

5) [symbol] indicates the binding position.}

Also, the compound represented by Formula 1 is represented by any one of the following compounds P-1 to P-110:

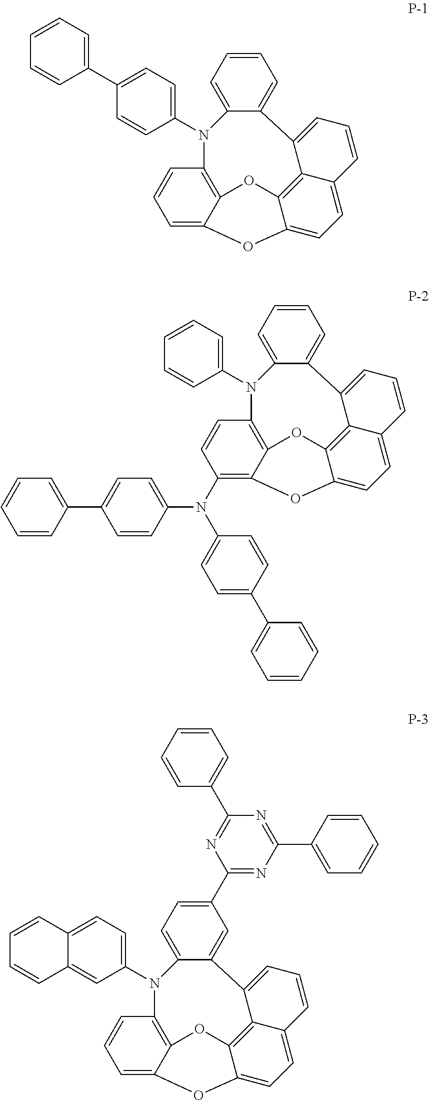 [SMILES: c1ccc(-c2ccc(N3c4ccccc4-c4cccc5ccc6c(c45)Oc4c(cccc43)O6)cc2)cc1 ; c1ccc(-c2ccc(N(c3ccc(-c4ccccc4)cc3)c3ccc4c5c3Oc3ccc6cccc(c6c3O5)-c3ccccc3N4c3ccccc3)cc2)cc1 ; c1ccc(-c2nc(-c3ccccc3)nc(-c3ccc4c(c3)-c3cccc5ccc6c(c35)Oc3c(cccc3N4c3ccc4ccccc4c3)O6)n2)cc1]

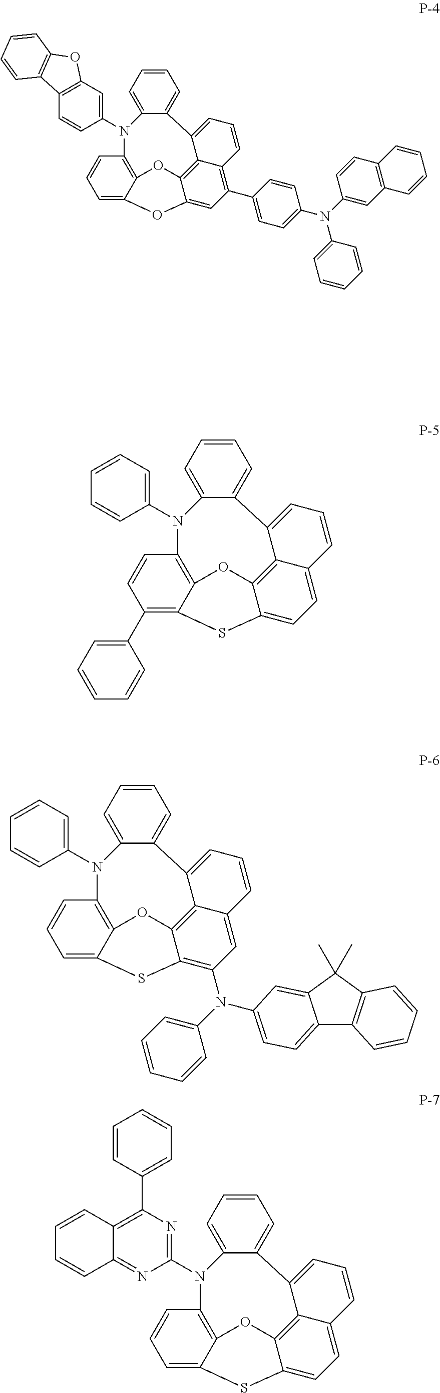

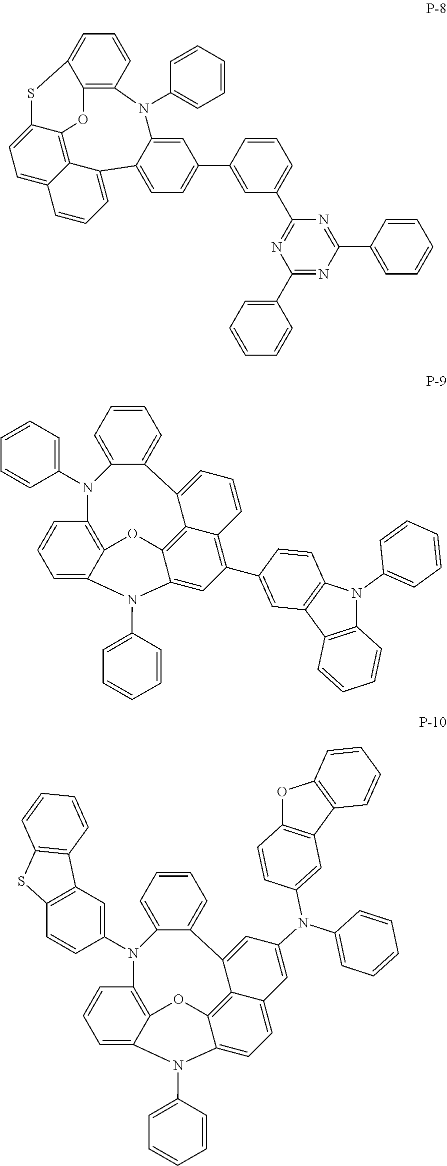 [SMILES: c1ccc(-c2nc(-c3ccccc3)nc(-c3cccc(-c4ccc5c(c4)N(c4ccccc4)c4cccc6c4Oc4c(ccc7cccc-5c47)S6)c3)n2)cc1 ; c1ccc(-n2c3ccccc3c3cc(-c4cc5c6oc7c(cccc7n5-c5ccccc5)n(-c5ccccc5)c5ccccc5c5cccc4c56)ccc32)cc1 ; c1ccc(N(c2ccc3oc4ccccc4c3c2)c2cc3ccc4c5oc6c(cccc6n4-c4ccccc4)n(-c4ccc6sc7ccccc7c6c4)c4ccccc4c(c2)c35)cc1]

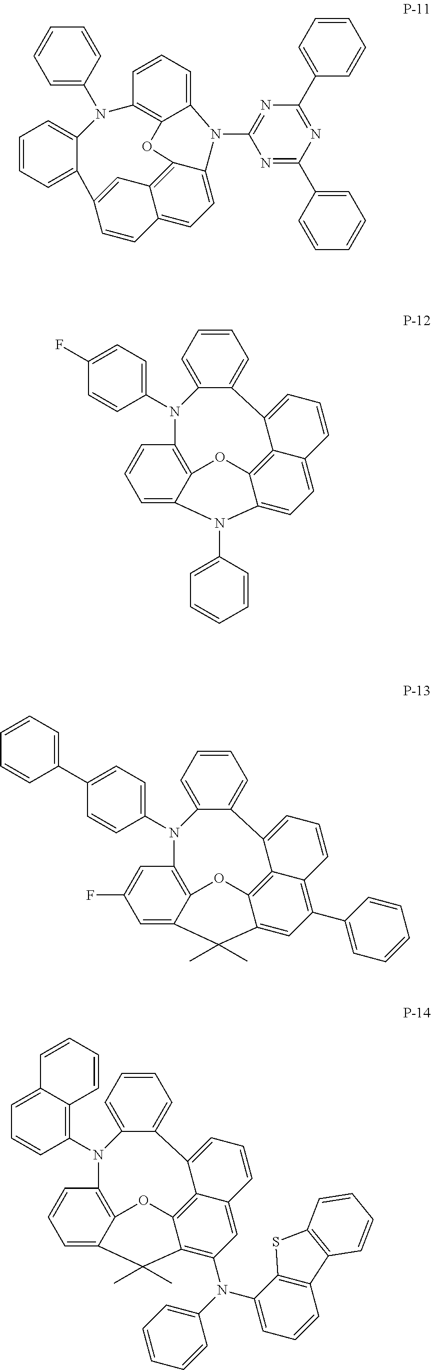 [SMILES: c1ccc(-c2nc(-c3ccccc3)nc(-n3c4ccc5ccc6cc5c4Oc4c3cccc4n(-c3ccccc3)c3ccccc63)n2)cc1 ; Fc1ccc(-n2c3ccccc3c3cccc4ccc5c(oc6c2cccc6n5-c2ccccc2)c43)cc1 ; CC1(C)c2cc(F)cc3c2Oc2c1cc(-c1ccccc1)c1cccc(c21)-c1ccccc1N3c1ccc(-c2ccccc2)cc1 ; CC1(C)c2cccc3c2Oc2c1c(N(c1ccccc1)c1cccc4c1sc1ccccc14)cc1cccc(c21)-c1ccccc1N3c1cccc2ccccc12]

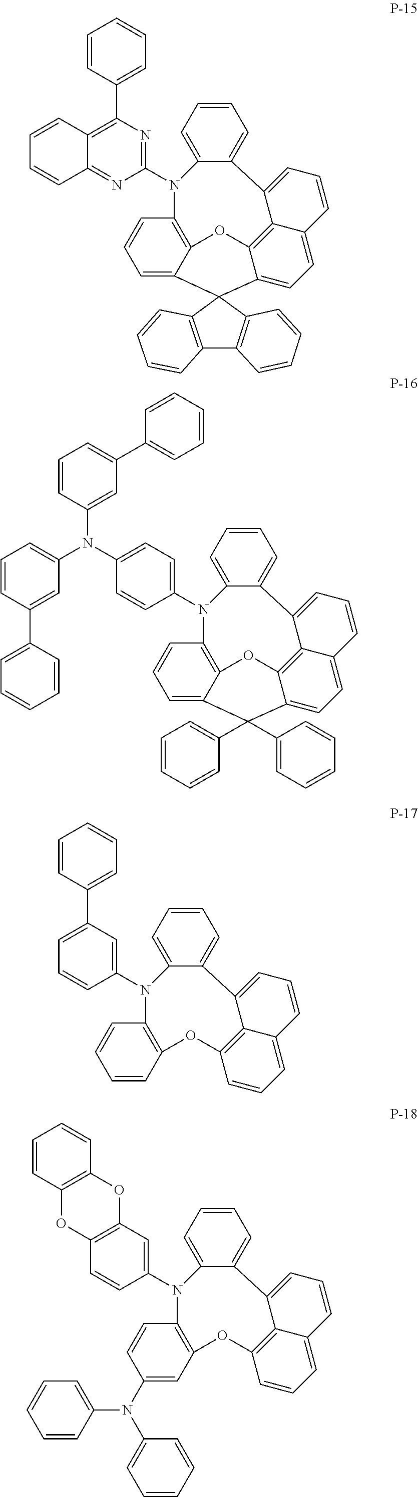

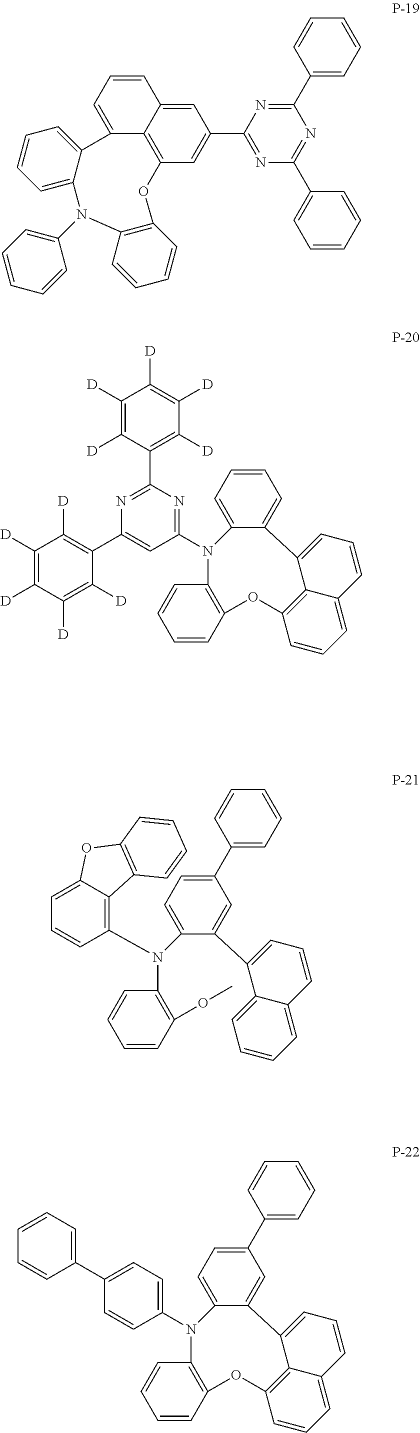 [SMILES: c1ccc(-c2nc(-c3ccccc3)nc(-c3cc4c5c(cccc5c3)-c3ccccc3N(c3ccccc3)c3ccccc3O4)n2)cc1 ; [2H]c1c([2H])c([2H])c(-c2cc(N3c4ccccc4Oc4cccc5cccc(c45)-c4ccccc43)nc(-c3c([2H])c([2H])c([2H])c([2H])c3[2H])n2)c([2H])c1[2H] ; COc1ccccc1N(c1ccc(-c2ccccc2)cc1-c1cccc2ccccc12)c1cccc2oc3ccccc3c12 ; c1ccc(-c2ccc(N3c4ccccc4Oc4cccc5cccc(c45)-c4cc(-c5ccccc5)ccc43)cc2)cc1]

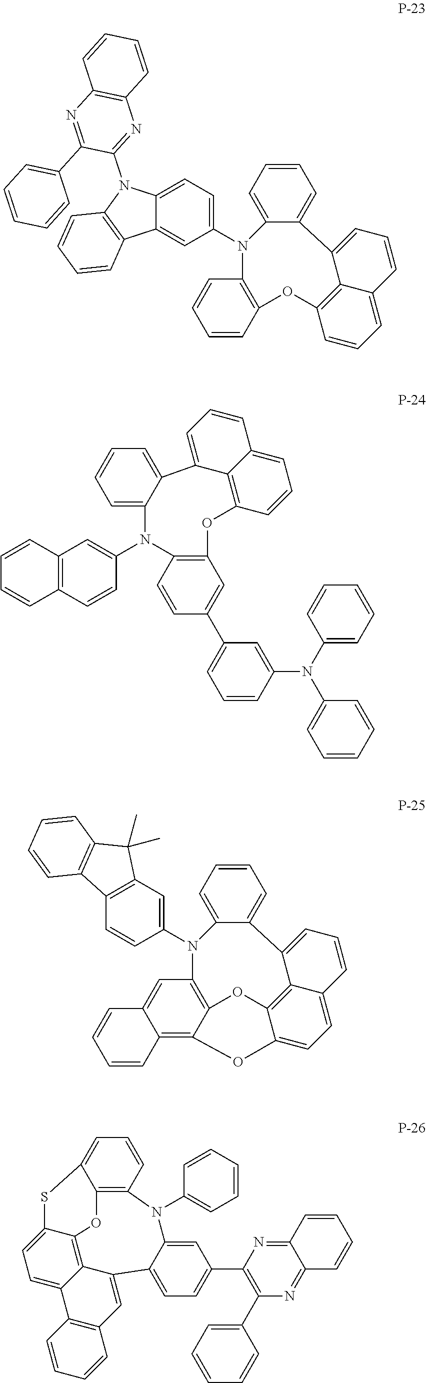 [SMILES: c1ccc(-c2nc3ccccc3nc2-n2c3ccccc3c3cc(N4c5ccccc5Oc5cccc6cccc(c56)-c5ccccc54)ccc32)cc1 ; c1ccc(N(c2ccccc2)c2cccc(-c3ccc4c(c3)Oc3cccc5cccc(c35)-c3ccccc3N4c3ccc4ccccc4c3)c2)cc1 ; CC1(C)c2ccccc2-c2ccc(N3c4ccccc4-c4cccc5ccc6c(c45)Oc4c3cc3ccccc3c4O6)cc21 ; c1ccc(-c2nc3ccccc3nc2-c2ccc3c(c2)N(c2ccccc2)c2cccc4c2Oc2c(ccc5c2c-3cc2ccccc25)S4)cc1]

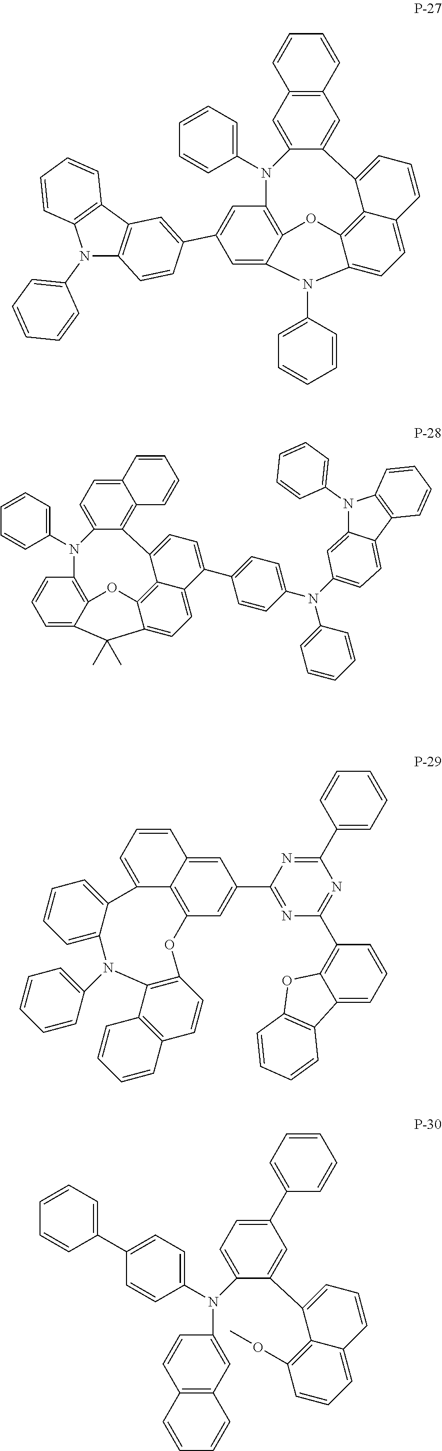 [SMILES: c1ccc(-n2c3ccccc3c3cc(-c4cc5c6oc7c8c(cccc8c8cc9ccccc9cc8n5-c5ccccc5)ccc7n(-c5ccccc5)c6c4)ccc32)cc1 ; CC1(C)c2cccc3c2Oc2c1ccc1c(-c4ccc(N(c5ccccc5)c5ccc6c7ccccc7n(-c7ccccc7)c6c5)cc4)ccc(c21)-c1c(ccc2ccccc12)N3c1ccccc1 ; c1ccc(-c2nc(-c3cc4c5c(cccc5c3)-c3ccccc3N(c3ccccc3)c3c(ccc5ccccc35)O4)nc(-c3cccc4c3oc3ccccc34)n2)cc1 ; COc1cccc2cccc(-c3cc(-c4ccccc4)ccc3N(c3ccc(-c4ccccc4)cc3)c3ccc4ccccc4c3)c12]

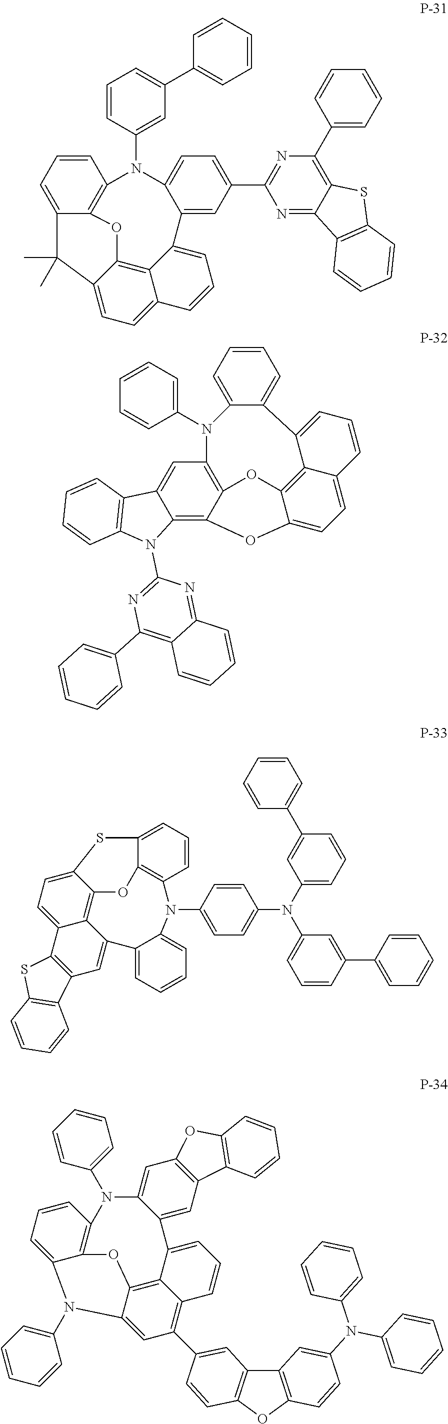

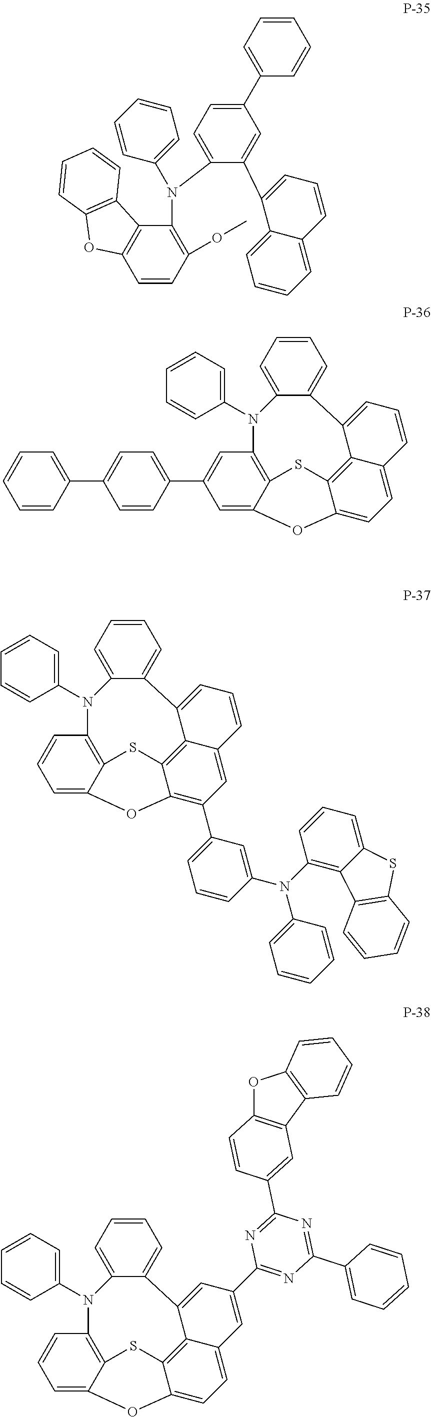

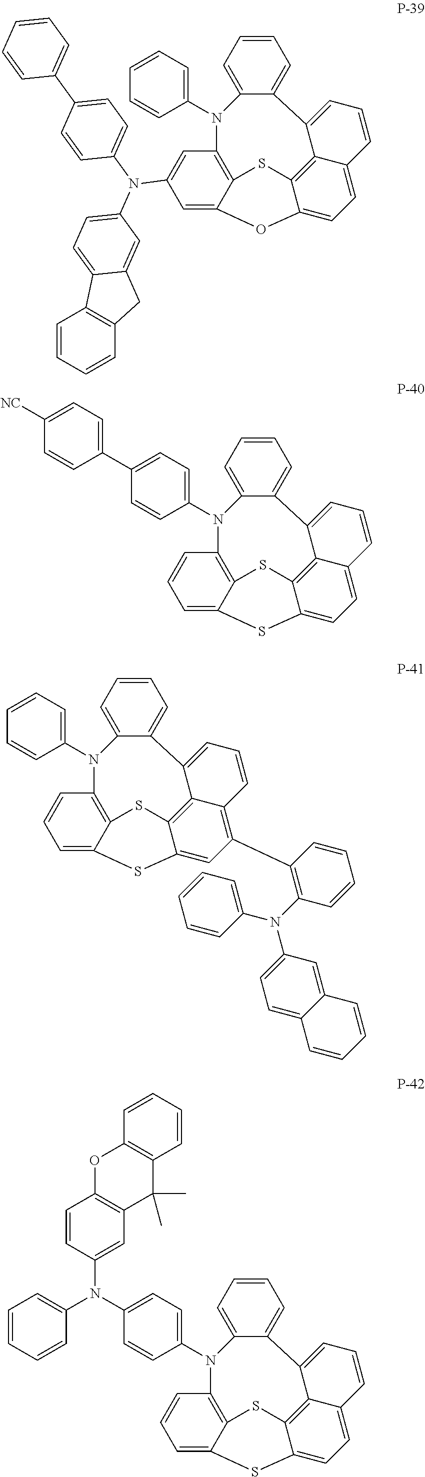

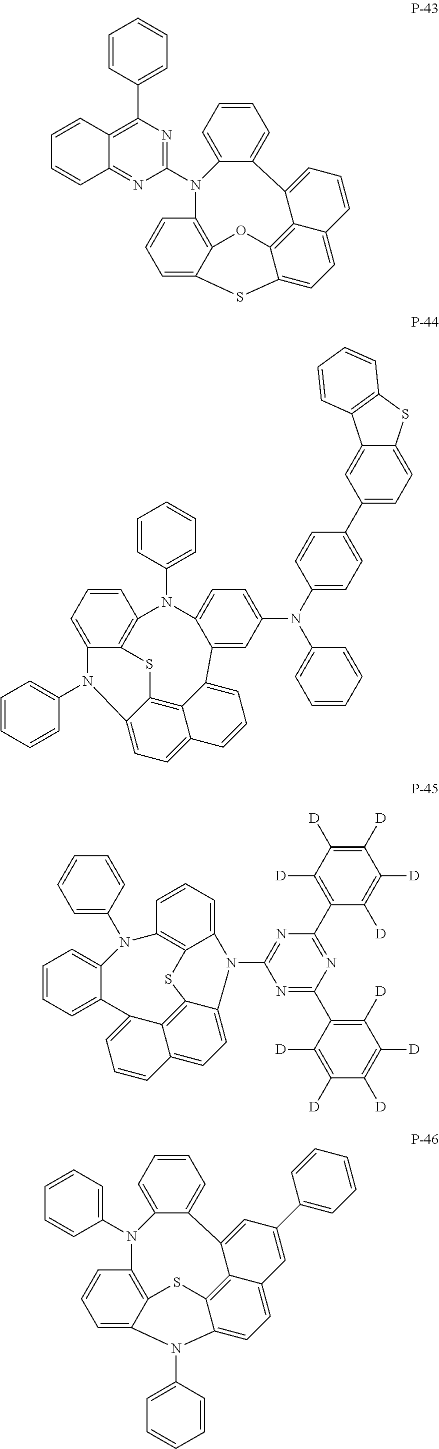

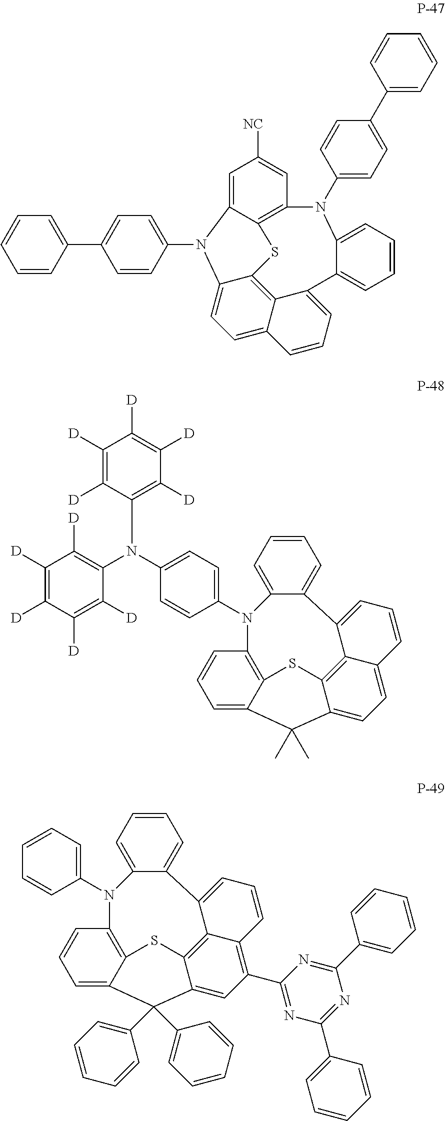 [SMILES: N#Cc1cc2c3sc4c5c(cccc5c5ccccc5n2-c2ccc(-c5ccccc5)cc2)ccc4n(-c2ccc(-c4ccccc4)cc2)c3c1 ; [2H]c1c([2H])c([2H])c(N(c2ccc(N3c4ccccc4-c4cccc5ccc6c(c45)Sc4c3cccc4C6(C)C)cc2)c2c([2H])c([2H])c([2H])c([2H])c2[2H])c([2H])c1[2H] ; c1ccc(-c2nc(-c3ccccc3)nc(-c3cc4c5c6c(cccc36)-c3ccccc3N(c3ccccc3)c3cccc(c3S5)C4(c3ccccc3)c3ccccc3)n2)cc1]

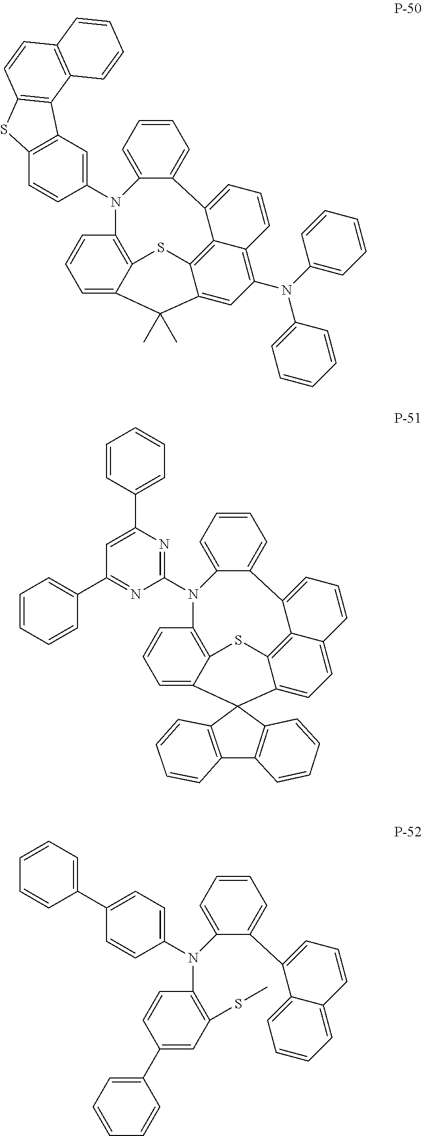 [SMILES: CC1(C)c2cccc3c2Sc2c1cc(N(c1ccccc1)c1ccccc1)c1cccc(c21)-c1ccccc1N3c1ccc2sc3ccc4ccccc4c3c2c1 ; c1ccc(-c2cc(-c3ccccc3)nc(N3c4ccccc4-c4cccc5ccc6c(c45)Sc4c3cccc4C63c4ccccc4-c4ccccc43)n2)cc1 ; CSc1cc(-c2ccccc2)ccc1N(c1ccc(-c2ccccc2)cc1)c1ccccc1-c1cccc2ccccc12]

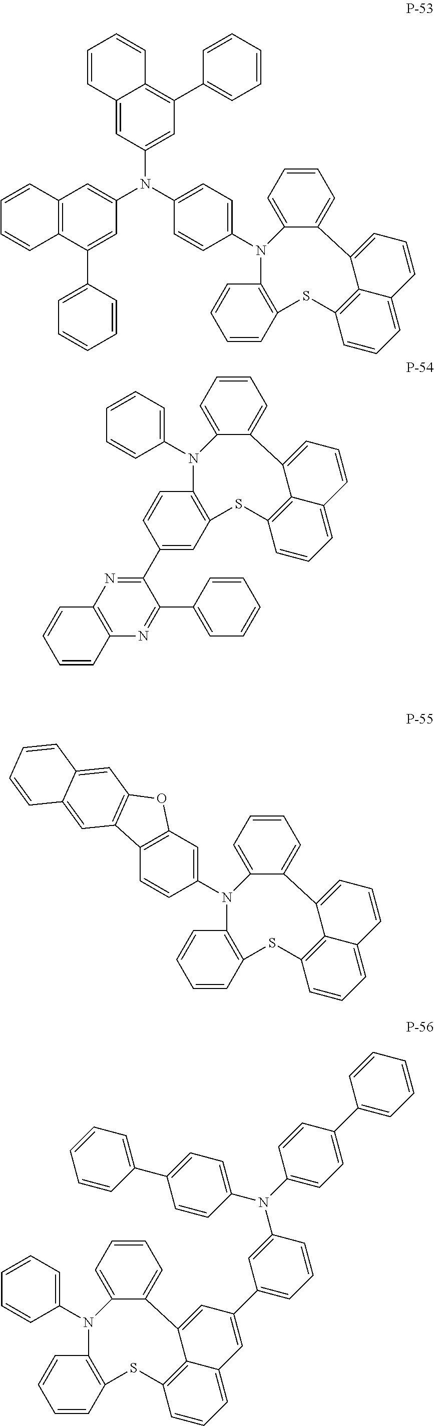

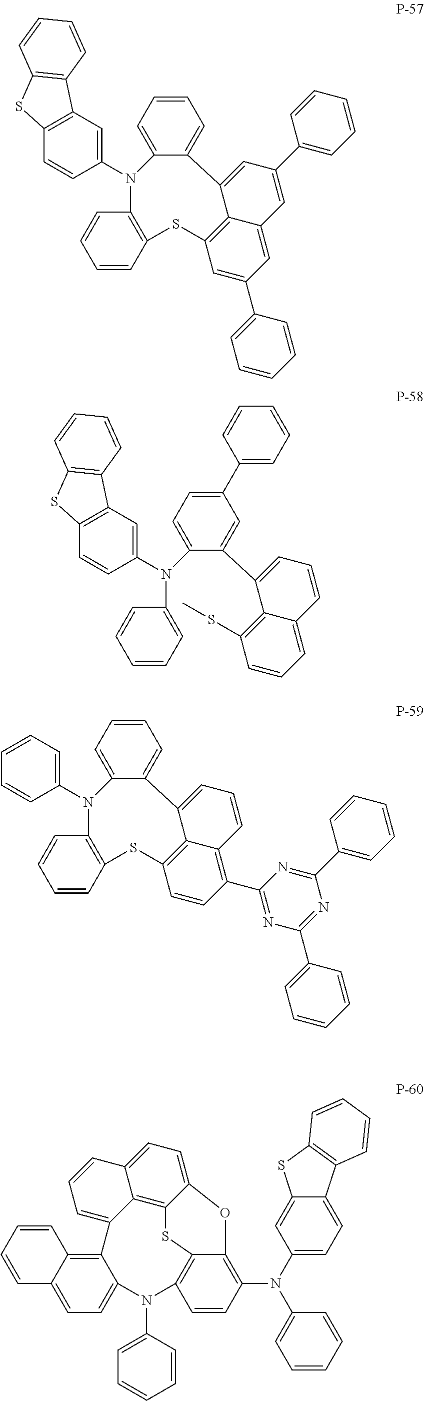

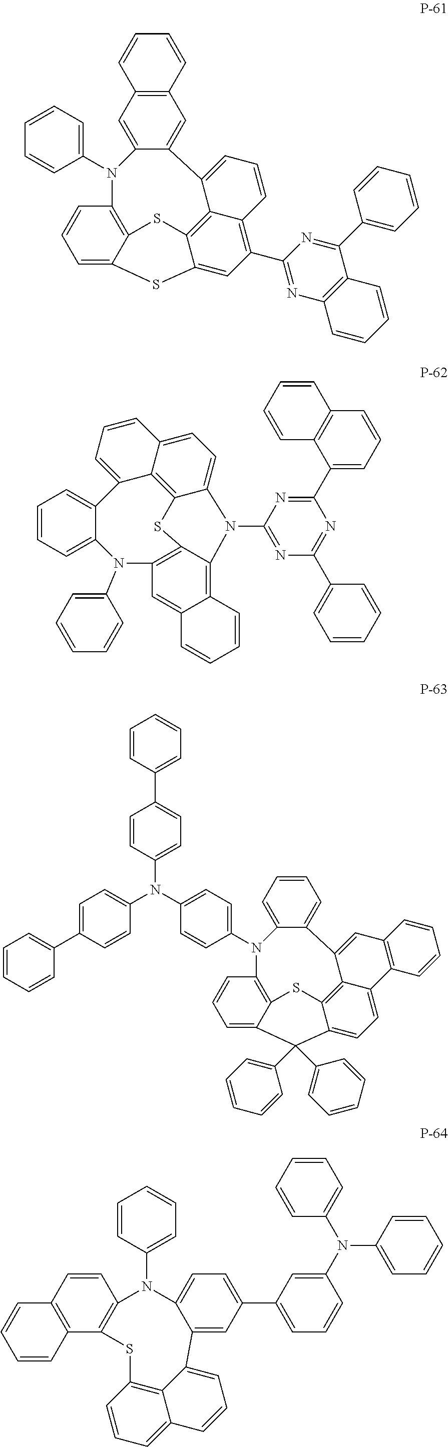

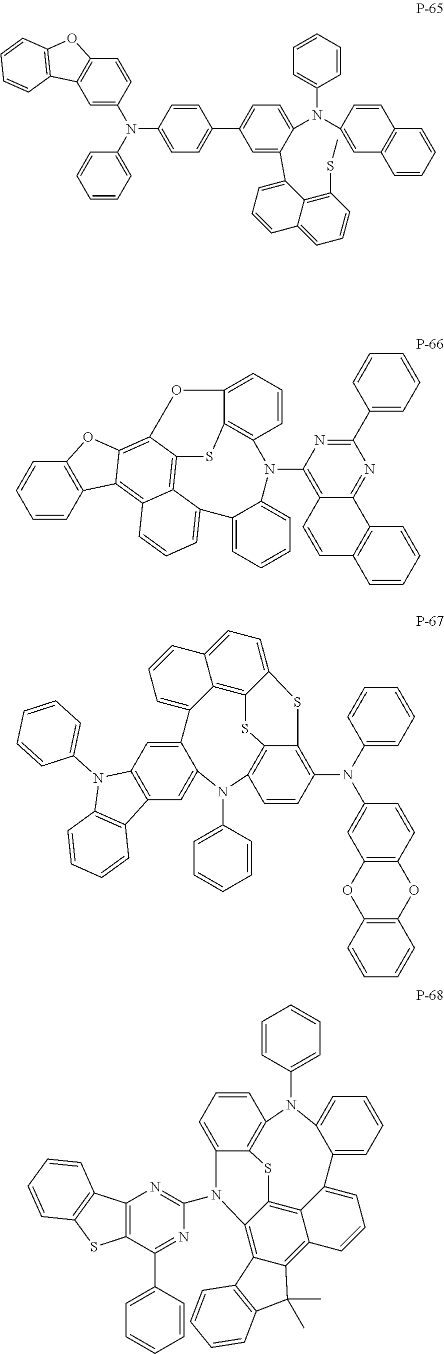 [SMILES: CSc1cccc2cccc(-c3cc(-c4ccc(N(c5ccccc5)c5ccc6oc7ccccc7c6c5)cc4)ccc3N(c3ccccc3)c3ccc4ccccc4c3)c12 ; c1ccc(-c2nc(N3c4ccccc4-c4cccc5c4c4c(c6oc7ccccc7c65)Oc5cccc3c5S4)c3ccc4ccccc4c3n2)cc1 ; c1ccc(N(c2ccc3c(c2)Oc2ccccc2O3)c2ccc3c4c2Sc2ccc5cccc(c5c2S4)-c2cc4c(cc2N3c2ccccc2)c2ccccc2n4-c2ccccc2)cc1 ; CC1(C)c2ccccc2-c2c1c1cccc3c4ccccc4n(-c4ccccc4)c4cccc5c4sc(c13)c2n5-c1nc(-c2ccccc2)c2sc3ccccc3c2n1]

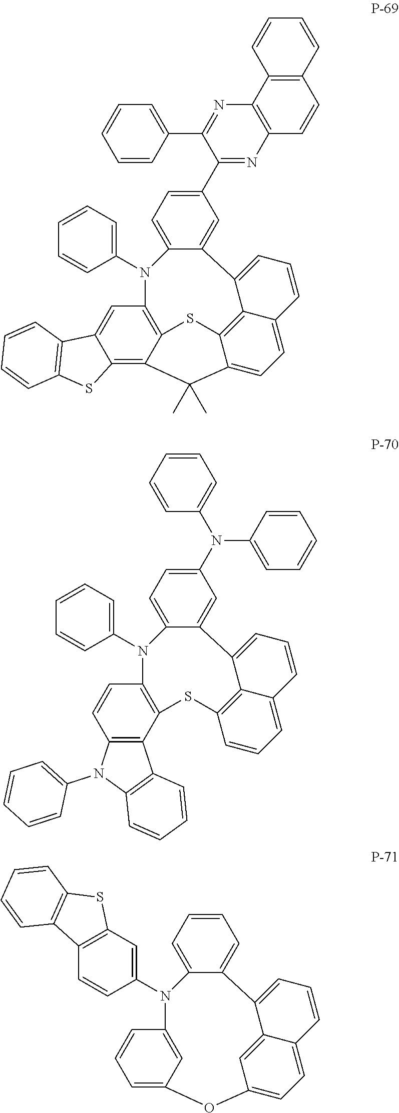

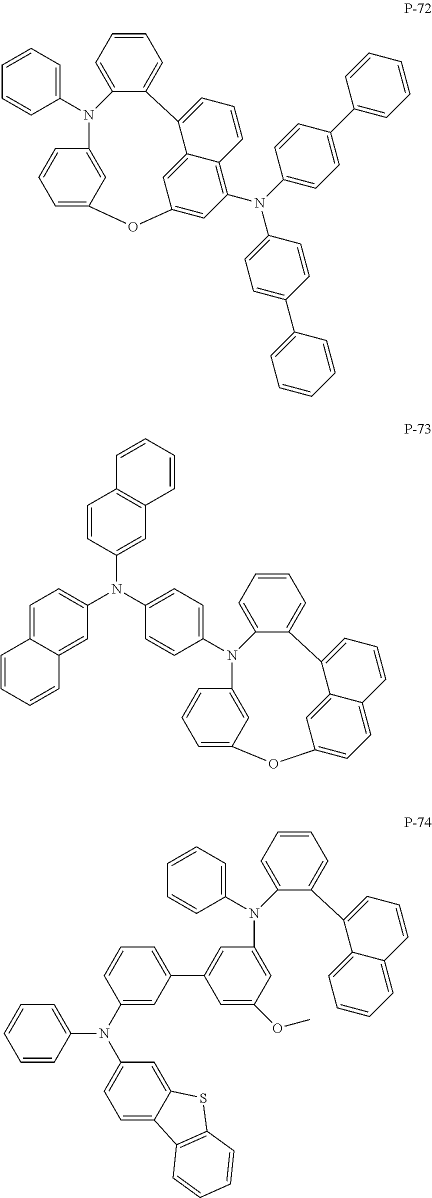 [SMILES: c1ccc(-c2ccc(N(c3ccc(-c4ccccc4)cc3)c3cc4cc5c(cccc35)-c3ccccc3N(c3ccccc3)c3cccc(c3)O4)cc2)cc1 ; c1cc2cc(c1)N(c1ccc(N(c3ccc4ccccc4c3)c3ccc4ccccc4c3)cc1)c1ccccc1-c1cccc3ccc(cc13)O2 ; COc1cc(-c2cccc(N(c3ccccc3)c3ccc4c(c3)sc3ccccc34)c2)cc(N(c2ccccc2)c2ccccc2-c2cccc3ccccc23)c1]

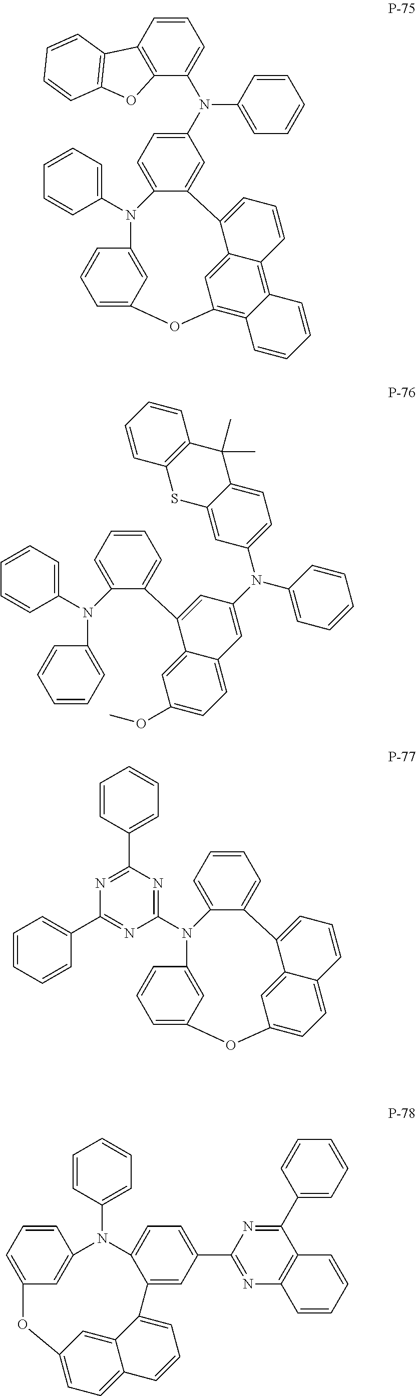

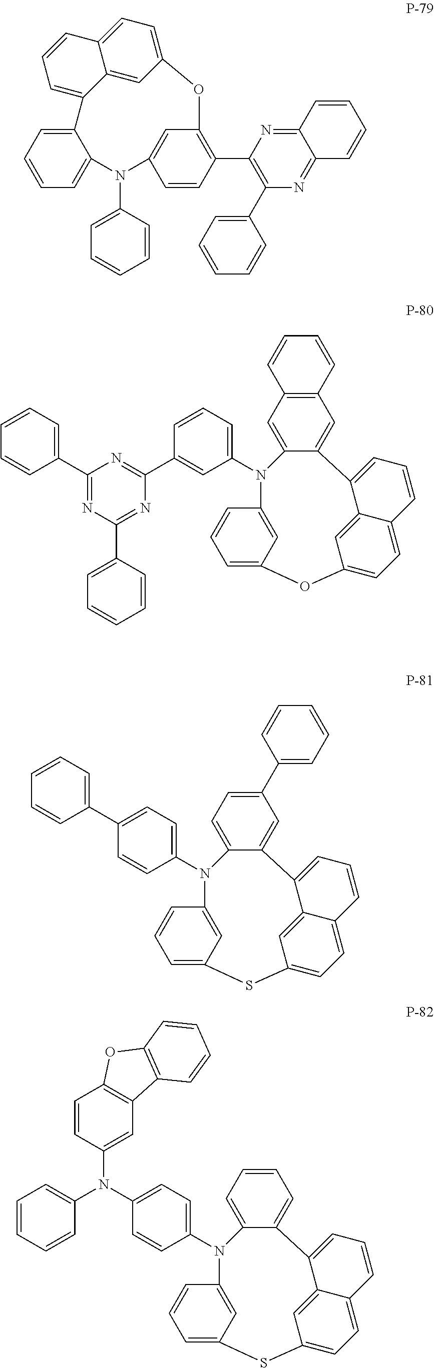 [SMILES: c1ccc(-c2nc3ccccc3nc2-c2ccc3cc2Oc2ccc4cccc(c4c2)-c2ccccc2N3c2ccccc2)cc1 ; c1ccc(-c2nc(-c3ccccc3)nc(-c3cccc(N4c5cccc(c5)Oc5ccc6cccc(c6c5)-c5cc6ccccc6cc54)c3)n2)cc1 ; c1ccc(-c2ccc(N3c4cccc(c4)Sc4ccc5cccc(c5c4)-c4cc(-c5ccccc5)ccc43)cc2)cc1 ; c1ccc(N(c2ccc(N3c4cccc(c4)Sc4ccc5cccc(c5c4)-c4ccccc43)cc2)c2ccc3oc4ccccc4c3c2)cc1]

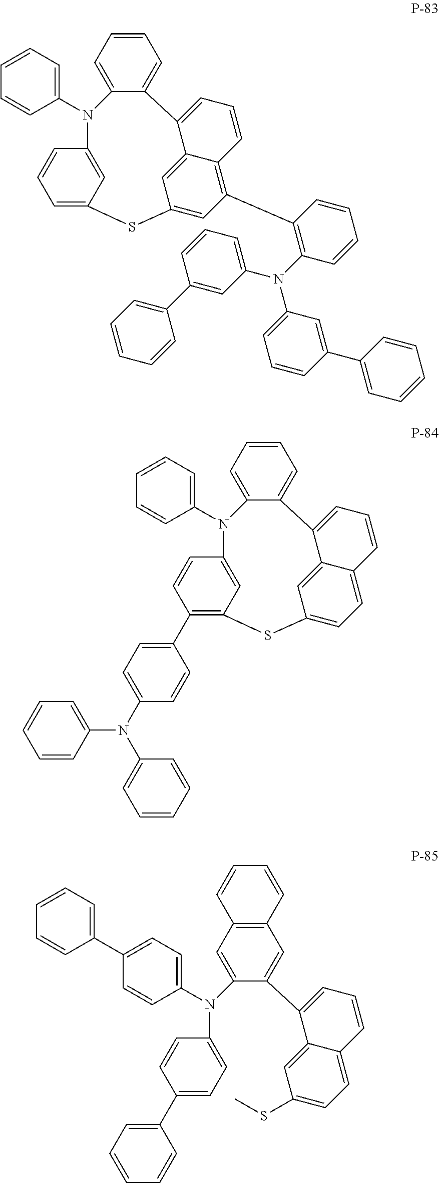 [SMILES: c1ccc(-c2cccc(N(c3cccc(-c4ccccc4)c3)c3ccccc3-c3cc4cc5c(cccc35)-c3ccccc3N(c3ccccc3)c3cccc(c3)S4)c2)cc1 ; c1ccc(N(c2ccccc2)c2ccc(-c3ccc4cc3Sc3ccc5cccc(c5c3)-c3ccccc3N4c3ccccc3)cc2)cc1 ; CSc1ccc2cccc(-c3cc4ccccc4cc3N(c3ccc(-c4ccccc4)cc3)c3ccc(-c4ccccc4)cc3)c2c1]

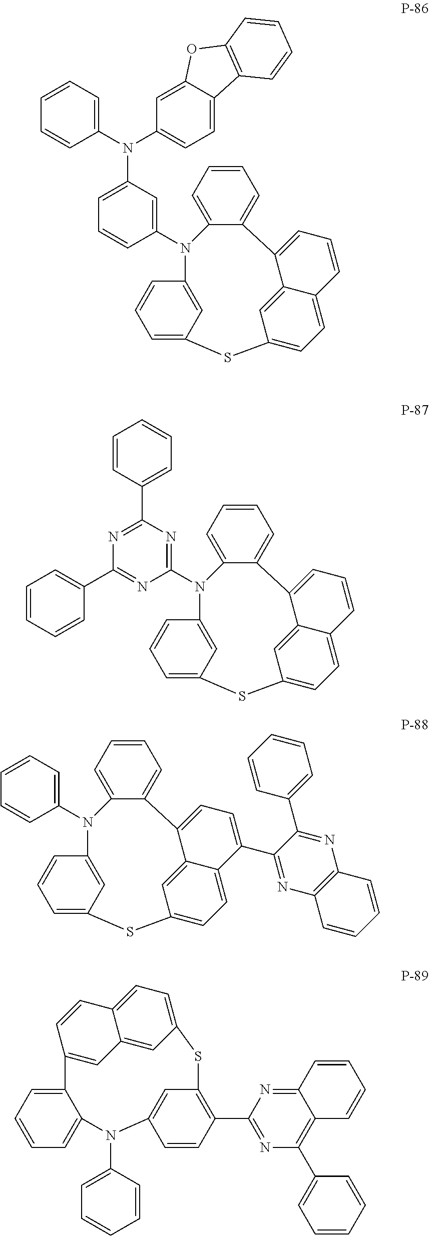 [SMILES: c1ccc(N(c2cccc(N3c4cccc(c4)Sc4ccc5cccc(c5c4)-c4ccccc43)c2)c2ccc3c(c2)oc2ccccc23)cc1 ; c1ccc(-c2nc(-c3ccccc3)nc(N3c4cccc(c4)Sc4ccc5cccc(c5c4)-c4ccccc43)n2)cc1 ; c1ccc(-c2nc3ccccc3nc2-c2ccc3c4cc(ccc24)Sc2cccc(c2)N(c2ccccc2)c2ccccc2-3)cc1 ; c1ccc(-c2nc(-c3ccc4cc3Sc3ccc5ccc(cc5c3)-c3ccccc3N4c3ccccc3)nc3ccccc23)cc1]

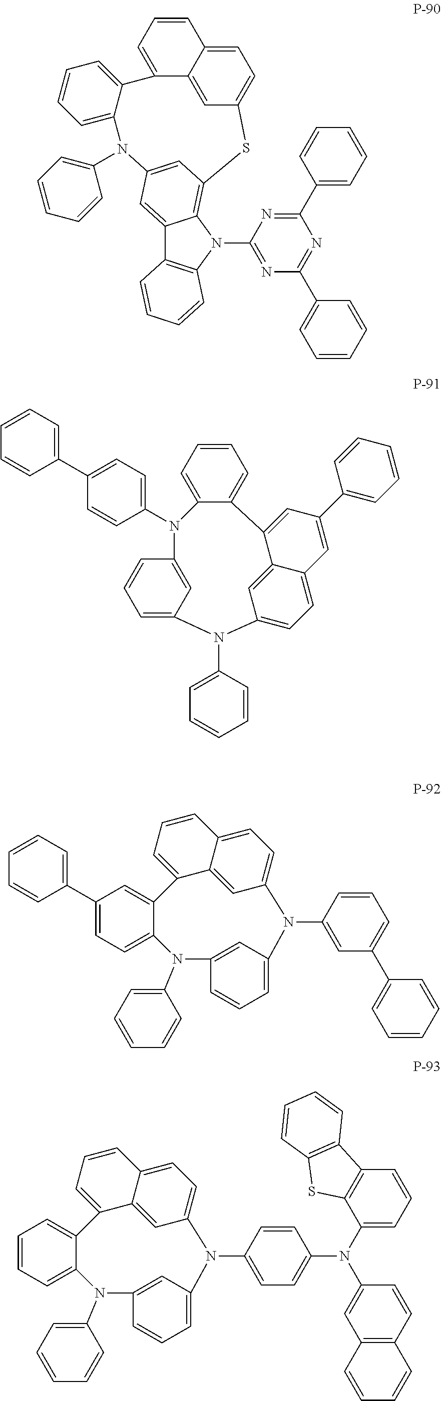 [SMILES: c1ccc(-c2nc(-c3ccccc3)nc(-n3c4ccccc4c4cc5cc(c43)Sc3ccc4cccc(c4c3)-c3ccccc3N5c3ccccc3)n2)cc1 ; c1ccc(-c2ccc(-n3c4cccc(c4)n(-c4ccccc4)c4ccc5cc(-c6ccccc6)cc(c5c4)c4ccccc43)cc2)cc1 ; c1ccc(-c2cccc(-n3c4ccc5cccc(c5c4)c4cc(-c5ccccc5)ccc4n(-c4ccccc4)c4cccc3c4)c2)cc1 ; c1ccc(-n2c3cccc(c3)n(-c3ccc(N(c4ccc5ccccc5c4)c4cccc5c4sc4ccccc45)cc3)c3ccc4cccc(c4c3)c3ccccc32)cc1]

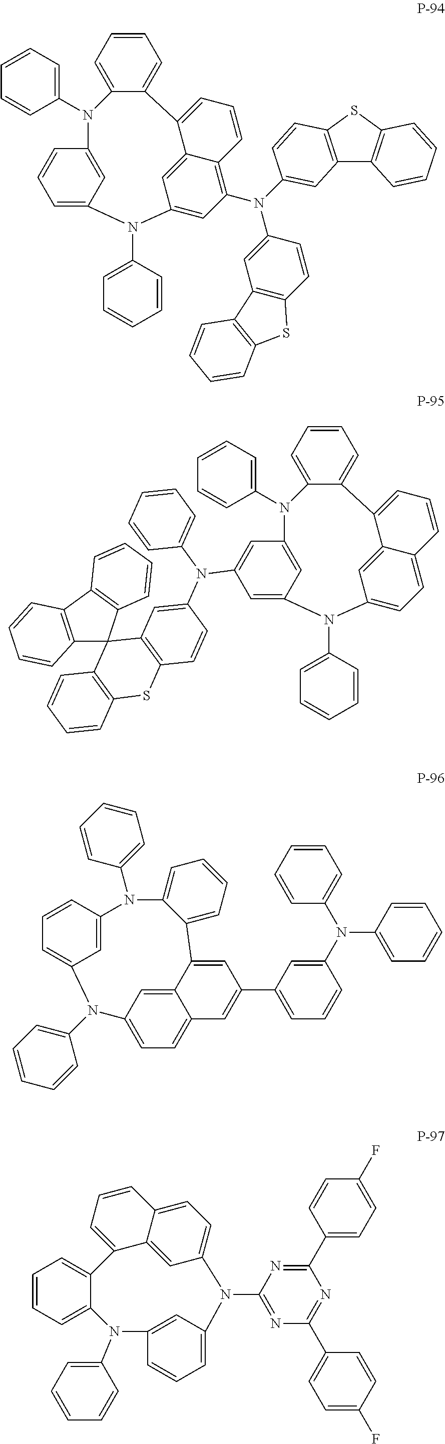 [SMILES: c1ccc(-n2c3cc(N(c4ccc5sc6ccccc6c5c4)c4ccc5sc6ccccc6c5c4)c4cccc(c4c3)c3ccccc3n(-c3ccccc3)c3cccc2c3)cc1 ; c1ccc(N(c2ccc3c(c2)C2(c4ccccc4S3)c3ccccc3-c3ccccc32)c2cc3cc(c2)n(-c2ccccc2)c2ccccc2c2cccc4ccc(cc42)n3-c2ccccc2)cc1 ; c1ccc(N(c2ccccc2)c2cccc(-c3cc4ccc5cc4c(c3)c3ccccc3n(-c3ccccc3)c3cccc(c3)n5-c3ccccc3)c2)cc1 ; Fc1ccc(-c2nc(-c3ccc(F)cc3)nc(-n3c4ccc5cccc(c5c4)c4ccccc4n(-c4ccccc4)c4cccc3c4)n2)cc1]

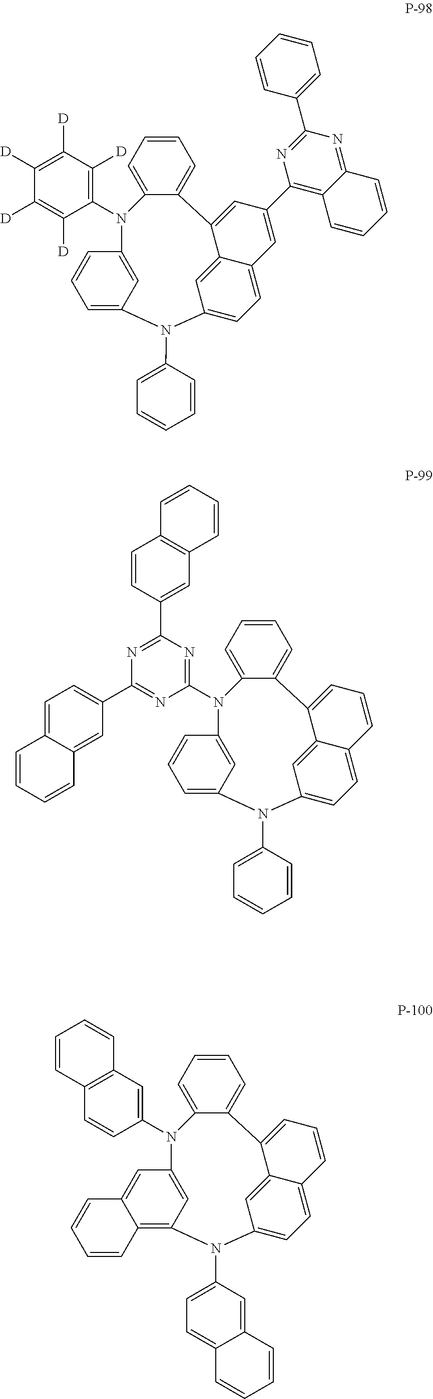 [SMILES: [2H]c1c([2H])c([2H])c(-n2c3cccc(c3)n(-c3ccccc3)c3ccc4cc(-c5nc(-c6ccccc6)nc6ccccc56)cc(c4c3)c3ccccc32)c([2H])c1[2H] ; c1ccc(-n2c3ccc4cccc(c4c3)c3ccccc3n(-c3nc(-c4ccc5ccccc5c4)nc(-c4ccc5ccccc5c4)n3)c3cccc2c3)cc1 ; c1ccc2cc(-n3c4ccc5cccc(c5c4)c4ccccc4n(-c4ccc5ccccc5c4)c4cc5ccccc5c3c4)ccc2c1]

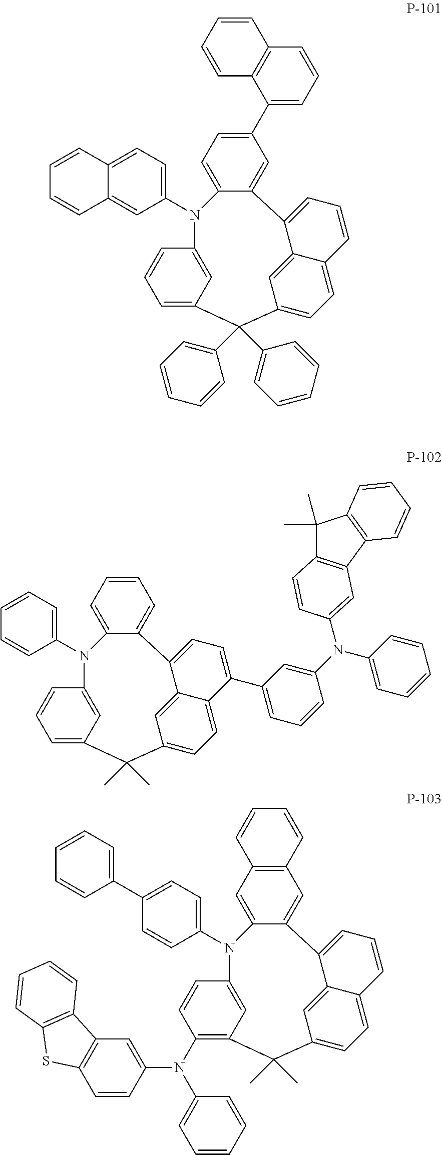 [SMILES: c1ccc(C2(c3ccccc3)c3cccc(c3)N(c3ccc4ccccc4c3)c3ccc(-c4cccc5ccccc45)cc3-c3cccc4ccc2cc34)cc1 ; CC1(C)c2cccc(c2)N(c2ccccc2)c2ccccc2-c2ccc(-c3cccc(N(c4ccccc4)c4ccc5c(c4)-c4ccccc4C5(C)C)c3)c3ccc1cc23 ; CC1(C)c2ccc3cccc(c3c2)-c2cc3ccccc3cc2N(c2ccc(-c3ccccc3)cc2)c2ccc(N(c3ccccc3)c3ccc4sc5ccccc5c4c3)c1c2]

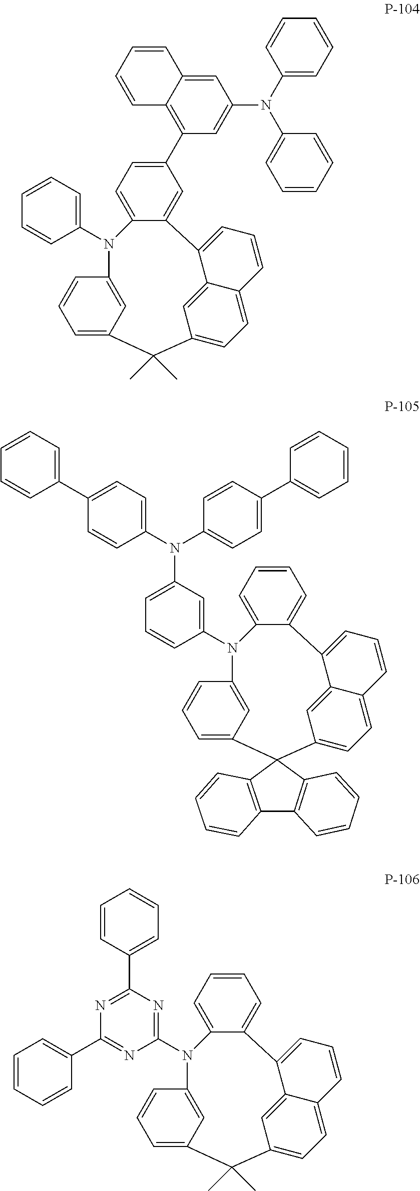 [SMILES: CC1(C)c2cccc(c2)N(c2ccccc2)c2ccc(-c3cc(N(c4ccccc4)c4ccccc4)cc4ccccc34)cc2-c2cccc3ccc1cc23 ; c1ccc(-c2ccc(N(c3ccc(-c4ccccc4)cc3)c3cccc(N4c5cccc(c5)C5(c6ccc7cccc(c7c6)-c6ccccc64)c4ccccc4-c4ccccc45)c3)cc2)cc1 ; CC1(C)c2cccc(c2)N(c2nc(-c3ccccc3)nc(-c3ccccc3)n2)c2ccccc2-c2cccc3ccc1cc23]

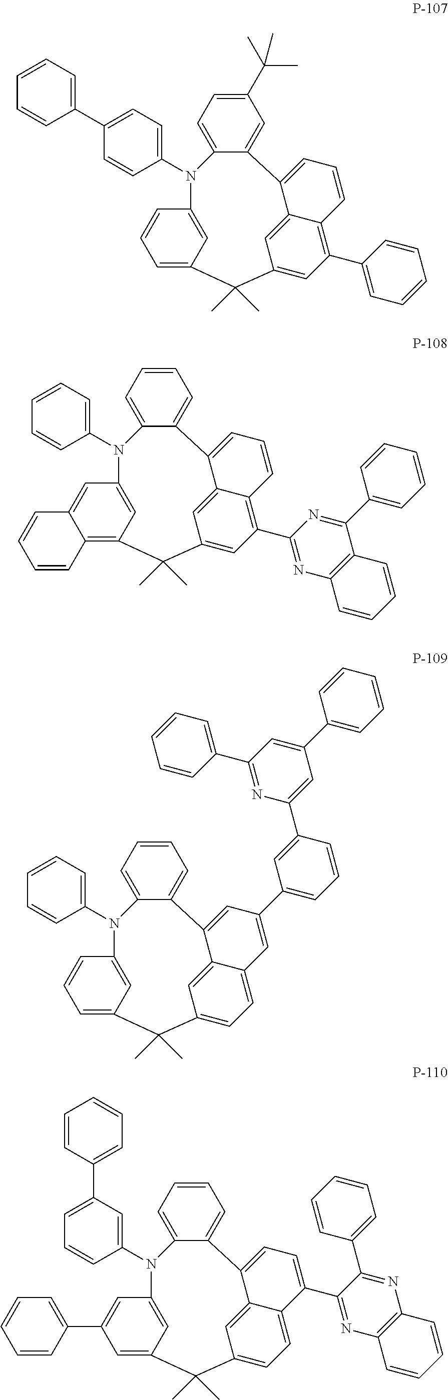 [SMILES: CC(C)(C)c1ccc2c(c1)-c1cccc3c(-c4ccccc4)cc(cc13)C(C)(C)c1cccc(c1)N2c1ccc(-c2ccccc2)cc1 ; CC1(C)c2cc(-c3nc(-c4ccccc4)c4ccccc4n3)c3cccc(c3c2)-c2ccccc2N(c2ccccc2)c2cc1c1ccccc1c2 ; CC1(C)c2cccc(c2)N(c2ccccc2)c2ccccc2-c2cc(-c3cccc(-c4cc(-c5ccccc5)cc(-c5ccccc5)n4)c3)cc3ccc1cc23 ; CC1(C)c2cc(-c3ccccc3)cc(c2)N(c2cccc(-c3ccccc3)c2)c2ccccc2-c2ccc(-c3nc4ccccc4nc3-c3ccccc3)c3ccc1cc23]

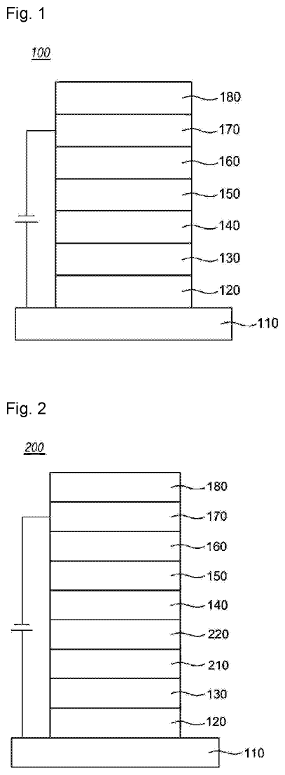

Referring to FIG. 1, the organic electronic element (100) according to the present invention includes a first electrode (110), a second electrode (170), and an organic material layer including a single compound or 2 or more compounds represented by Formula 1 between the first electrode (110) and the second electrode (170). In this case, the first electrode (110) may be an anode, and the second electrode (170) may be a cathode. In the case of an inverted type, the first electrode may be a cathode and the second electrode may be an anode.

The organic material layer may sequentially include a hole injection layer (120), a hole transport layer (130), an emitting layer (140), an electron transport layer (150), and an electron injection layer (160) on the first electrode (110). In this case, the remaining layers except for the emitting layer (140) may not be formed. It may further include a hole blocking layer, an electron blocking layer, an emitting-auxiliary layer (220), a buffer layer (210), etc. and the electron transport layer (150) and the like may serve as a hole blocking layer. (See FIG. 2)

Also, the organic electronic element according to an embodiment of the present invention may further include a protective layer or a light efficiency enhancing layer (180). The light efficiency enhancing layer may be formed on one of both surfaces of the first electrode not in contact with the organic material layer or on one of both surfaces of the second electrode not in contact with the organic material layer. The compound according to an embodiment of the present invention applied to the organic material layer may be used as a host or dopant of the hole injection layer (120), the hole transport layer (130), the emitting-auxiliary layer (220), electron transport auxiliary layer, the electron transport layer (150), and an electron injection layer (160), the emitting layer (140) or as a material for the light efficiency enhancing layer. Preferably, for example, the compound according to Formula 1 of the present invention may be used as a material of the emitting-auxiliary layer or the emitting layer.

The organic material layer may include 2 or more stacks including a hole transport layer, an emitting layer and an electron transport layer sequentially formed on the anode, further include a charge generation layer formed between the 2 or more stacks (see FIG. 3).

Otherwise, even with the same core, the band gap, electrical characteristics, interface characteristics, etc. may vary depending on which position the substituent is bonded to, therefore the choice of core and the combination of sub-substituents bound thereto are also very important, and in particular, when the optimal combination of energy levels and T1 values and unique properties of materials (mobility, interfacial characteristics, etc.) of each organic material layer is achieved, a long lifespan and high efficiency can be achieved at the same time.

The organic electroluminescent device according to an embodiment of the present invention may be manufactured using a PVD (physical vapor deposition) method. For example, depositing a metal or a metal oxide having conductivity or an alloy thereof on a substrate to form an anode, and after forming an organic material layer including the hole injection layer (120), the hole transport layer (130), the emitting layer (140), the electron transport layer (150) and the electron injection layer (160) thereon, it can be prepared by depositing a material that can be used as a cathode thereon.

Also, in the present invention, the organic material layer is formed by any one of a spin coating process, a nozzle printing process, an inkjet printing process, a slot coating process, a dip coating process, and a roll-to-roll process, and the organic material layer provides an organic electronic element comprising the compound as an electron transport material.

As another specific example, the same or different compounds of the compound represented by Formula 1 are mixed and used in the organic material layer.

Also, the present invention provides an emitting layer composition comprising the compound represented by Formula 1, and provides an organic electronic element including the emitting layer.

Also, the present invention provides an electronic device comprising a display device including the organic electronic element; and a control unit for driving the display device; In another aspect, the organic electronic element is at least one of an organic electroluminescent device, an organic solar cell, an organic photoreceptor, an organic transistor, and a device for monochromatic or white lighting. At this time, the electronic device may be a current or future wired/wireless communication terminal, and covers all kinds of electronic devices including mobile communication terminals such as mobile phones, a personal digital assistant (PDA), an electronic dictionary, a point-to-multipoint (PMP), a remote controller, a navigation unit, a game player, various kinds of TVs, and various kinds of computers.

Hereinafter, a synthesis example of the compound represented by Formula 1 of the present invention and a manufacturing example of an organic electronic element of the present invention will be described in detail with reference to Examples, but the present invention is not limited to the following Examples.

### Synthesis Example 1

The compound represented by Formula 1 according to the present invention (Final product) may be prepared by reacting as shown in Scheme 1 below, but is not limited thereto. Hal¹ is Cl, Br or I.

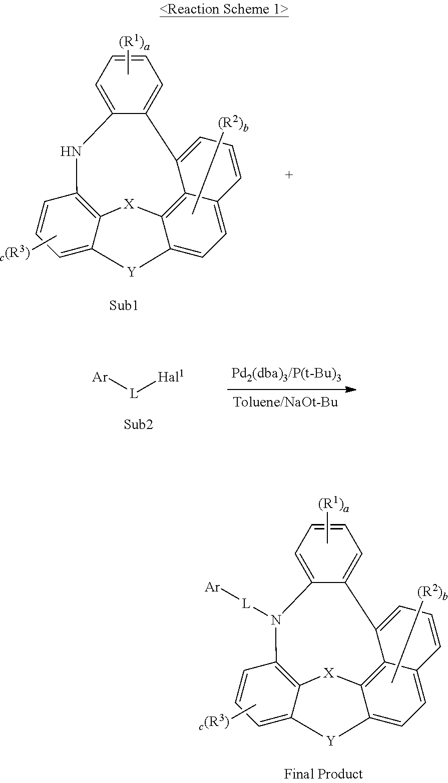

1. Synthesis of Sub 1

Sub 1 of Reaction Scheme 1 may be synthesized by the reaction route of Reaction Scheme 2 below, but is not limited thereto. Hal² is Cl, Br or I, and Hal³ is Br or I.

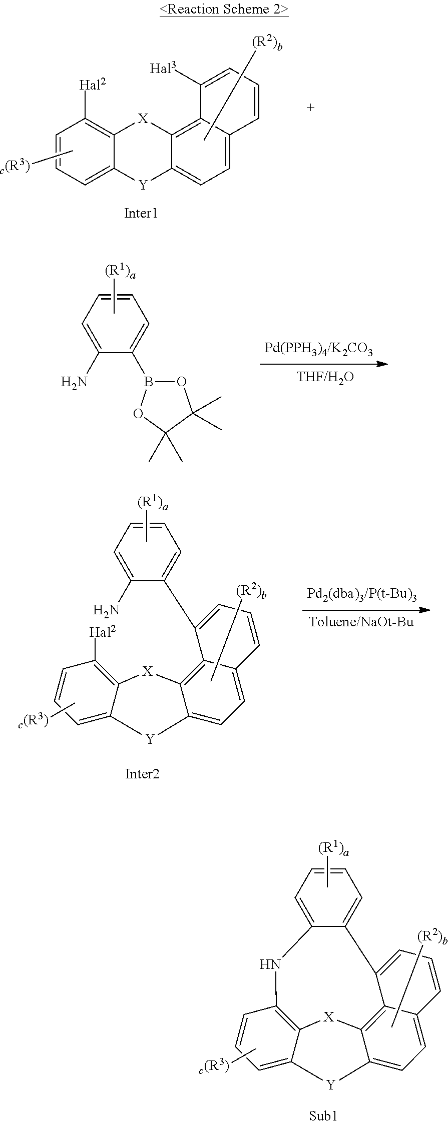

### 1. Synthesis Example of Sub1-1

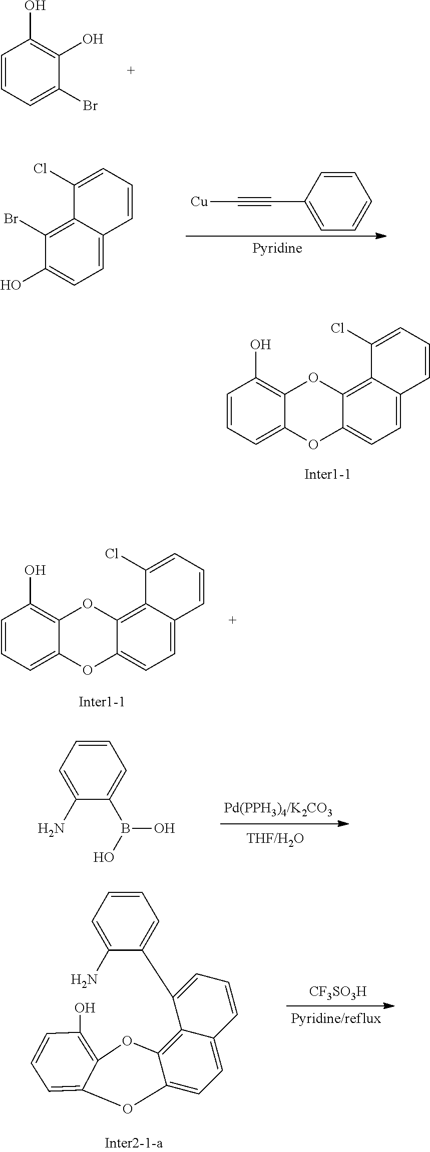

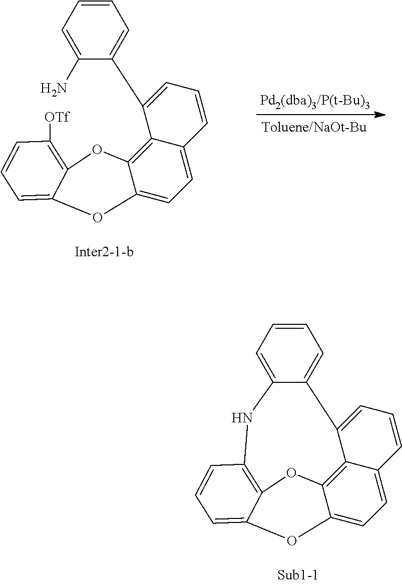 [SMILES: Nc1ccccc1-c1cccc2ccc3c(c12)Oc1c(cccc1OS(=O)(=O)C(F)(F)F)O3 ; "Pd2(dba)3/P(t-Bu)3" ; c1ccc2c(c1)Nc1cccc3c1Oc1c(ccc4cccc-2c14)O3]

(1) Synthesis of Inter1-1

Put 3-bromobenzene-1,2-diol (100 g, 529 mmol) and Cu(I) Phenylacetylenide (43.6 g, 265 mmol) in a round-bottom flask, and add pyridine (1.32 L). Thereafter, it was refluxed for 5 hours in a nitrogen atmosphere. After that, 1-bromo-8-chloronaphthalen-2-ol (68.1 g, 265 mmol) was added and further refluxed for 5 hours. After the reaction was completed, the product was concentrated and recrystallized by silicagel column to obtain 28.8 g of the product. (Yield 38%)

(2) Inter2-1-a

After dissolving Inter1-1 (12.0 g, 42.1 mmol) obtained in the above synthesis in THE (Tetrahydrofuran) (210 mL) in a round-bottom flask, (2-aminophenyl)boronic acid (5.8 g, 42.1 mmol), K₂CO₃ (17.5 g, 126 mmol), Pd(PPh₃)₄ (2.92 g, 2.53 mmol), and water (105 mL) were added and stirred at 80° C. When the reaction is complete, the mixture is extracted with CH₂Cl₂ and water, and the organic layer is dried over MgSO₄ and concentrated. Thereafter, the resulting organic material was recrystallized by silicagel column to obtain 10.9 g of a product. (yield 76%)

(3) Inter2-1-b

Inter2-1-a (10.9 g, 32.0 mmol) obtained in the above synthesis was added to an excess of trifluoromethane-sulfonic acid and stirred at room temperature for 24 hours, and water and pyridine (8:1) were slowly added and refluxed for 30 minutes. The temperature was lowered and the mixture was extracted with CH₂Cl₂ and water. After the organic material layer was dried over MgSO₄ and concentrated, the resulting product was subjected to silicagel column and recrystallization to obtain 11.5 g of the product. (yield 76%)

(4) Synthesis of Sub1-1

After dissolving Inter2-1-b (11.5 g, 24.3 mmol) obtained in the above synthesis in toluene (121 mL) in a round-bottom flask, Pd₂(dba)₃ (0.67 g, 0.73 mmol), P(t-Bu)₃ (0.29 g, 1.46 mmol), NaOt-Bu (4.7 g, 48.6 mmol) were added and the reaction proceeded at 80° C. When the reaction is complete, the mixture is extracted with CH₂Cl₂ and water, and the organic layer is dried over MgSO₄ and concentrated. Thereafter, the resulting organic material was recrystallized by silicagel column to obtain 5.7 g of a product. (yield 72%)

### 2. Synthesis Example of Sub1-2

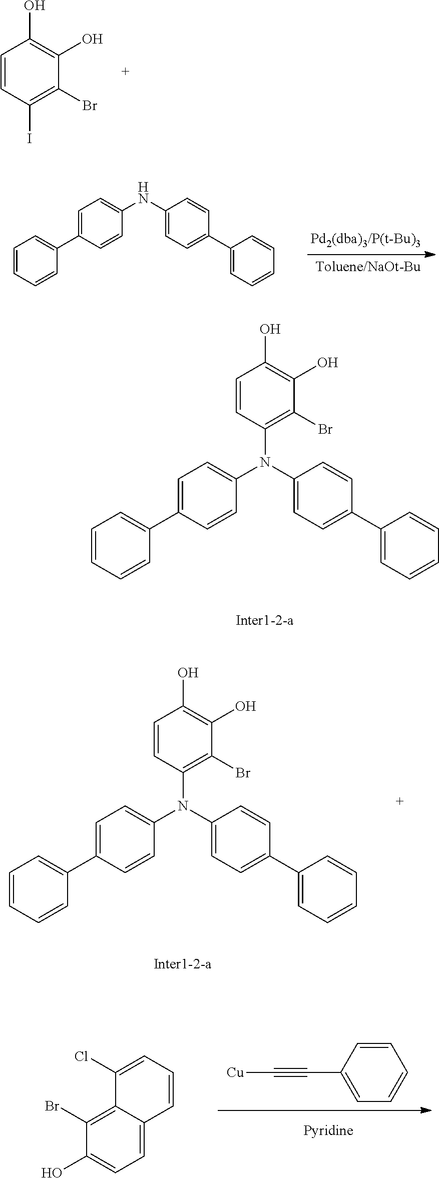

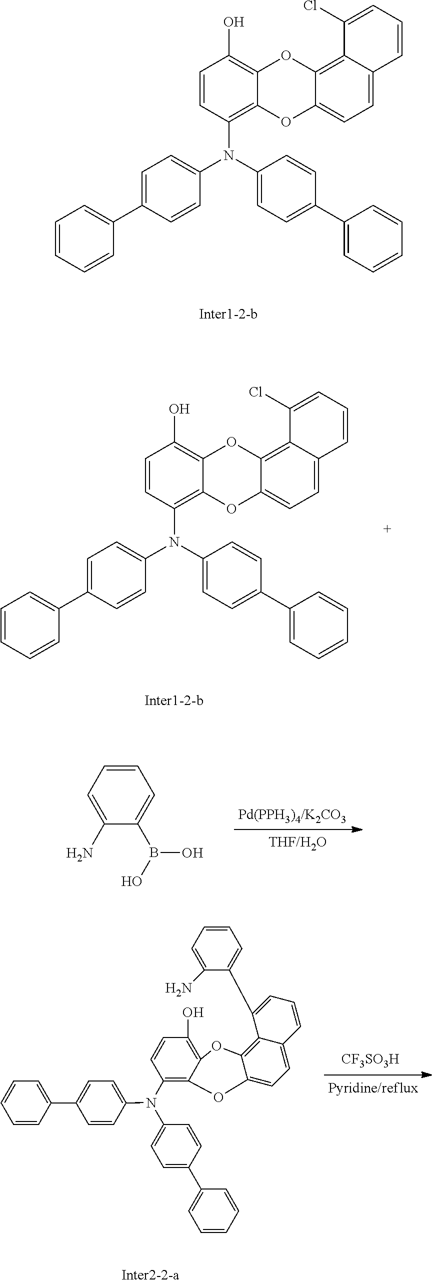

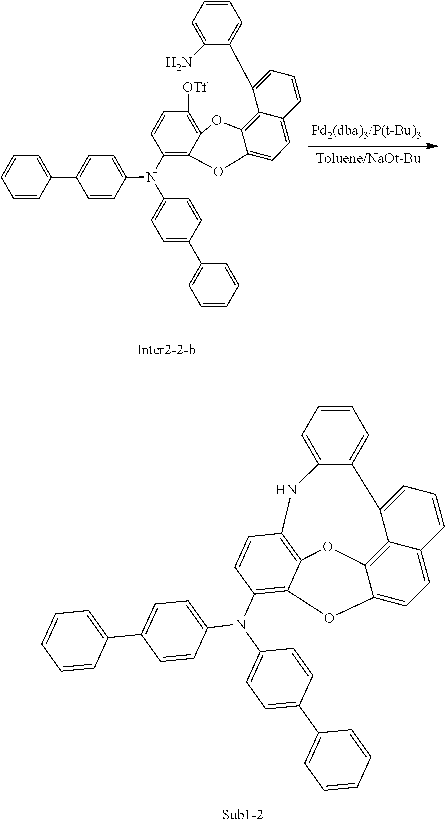 [SMILES: Nc1ccccc1-c1cccc2ccc3c(c12)Oc1c(OS(=O)(=O)C(F)(F)F)ccc(N(c2ccc(-c4ccccc4)cc2)c2ccc(-c4ccccc4)cc2)c1O3 ; c1ccc(-c2ccc(N(c3ccc(-c4ccccc4)cc3)c3ccc4c5c3Oc3ccc6cccc(c6c3O5)-c3ccccc3N4)cc2)cc1]

(1) Synthesis of Inter1-2-a

After dissolving 3-bromo-4-iodobenzene-1,2-diol (50.0 g, 159 mmol) in toluene (795 mL) in a round-bottom flask, di([1,1′-biphenyl]-4-yl)amine (51.0 g, 159 mmol), Pd₂(dba)₃ (4.36 g, 4.76 mmol), P(t-Bu)₃ (1.93 g, 9.53 mmol), NaOt-Bu (30.5 g, 318 mmol) were added and the reaction proceeded at 50° C. When the reaction is complete, the mixture is extracted with CH₂Cl₂ and water, and the organic layer is dried over MgSO₄ and concentrated. Thereafter, the resulting organic material was recrystallized by silicagel column to obtain 54.9 g of a product. (yield 68%)

(2) Synthesis of Inter1-2-b

The obtained Inter1-2-a (54.9 g, 109 mmol) and Cu(I) Phenylacetylenide (8.95 g, 54.3 mmol), 1-bromo-8-chloronaphthalen-2-ol (14.0 g, 54.3 mmol) were used for the synthesis of Inter1-1 to obtain 11.9 g of a product. (Yield 36%)

(3) Synthesis of Inter2-2-a

The obtained Inter1-2-b (11.9 g, 19.7 mmol), (2-aminophenyl)boronic acid (2.7 g, 19.7 mmol), K₂CO₃ (8.2 g, 59.0 mmol), Pd(PPh₃)₄ (1.36 g, 1.18 mmol) were used for the synthesis of Inter2-1-a to obtain 9.4 g of a product. (Yield 72%)

(4) Synthesis of Inter2-2-b

The obtained Inter2-2-a (9.4 g, 14.2 mmol) obtained in the above synthesis was used for the synthesis of Inter2-1-b to obtain 8.8 g of the product. (yield 78%)

(5) Synthesis of Sub1-2

The obtained Inter2-2-b (8.8 g, 11.1 mmol), Pd₂(dba)₃ (0.30 g, 0.33 mmol), P(t-Bu)₃ (0.13 g, 0.67 mmol), NaOt-Bu (2.1 g, 22.2 mmol) were used for the synthesis of Sub1-1 to obtain 4.6 g of the product. (Yield 65%)

### 3. Synthesis Example of Sub1-11

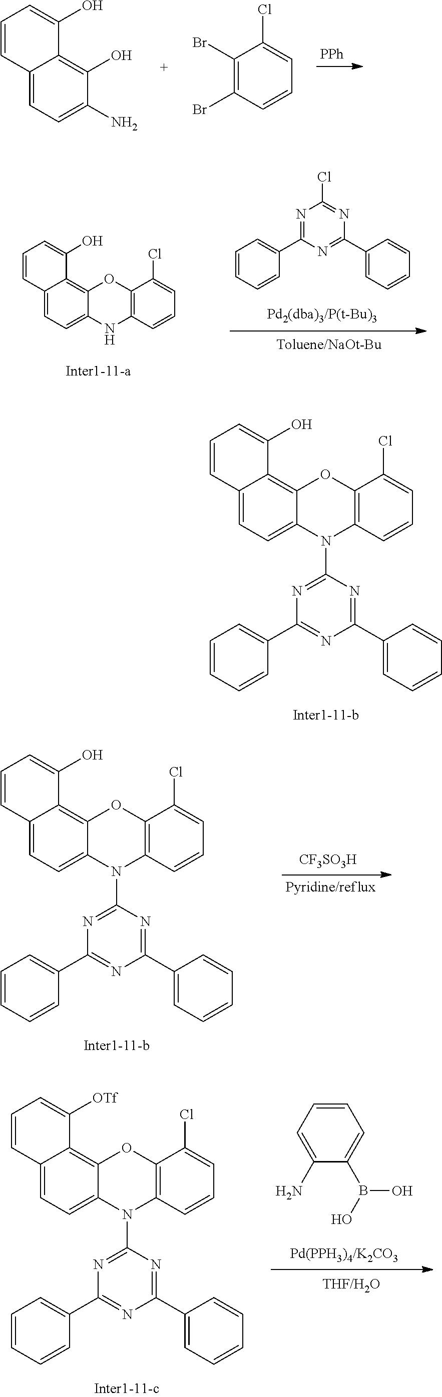

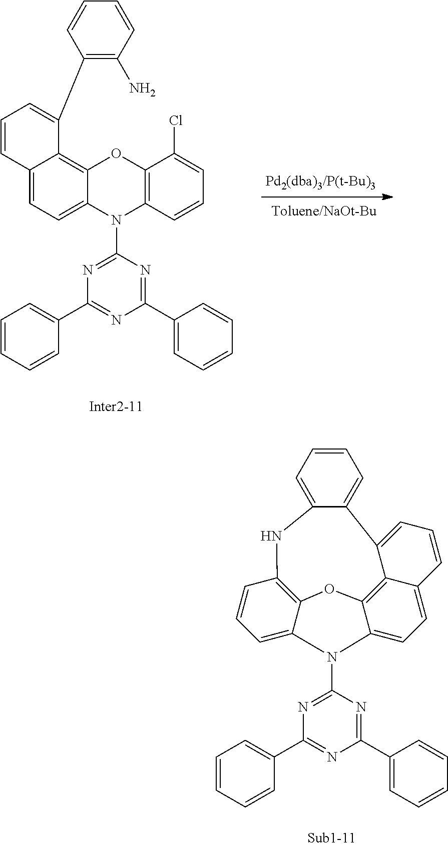 [SMILES: Nc1ccccc1-c1cccc2ccc3c(c12)Oc1c(Cl)cccc1N3c1nc(-c2ccccc2)nc(-c2ccccc2)n1 ; c1ccc(-c2nc(-c3ccccc3)nc(N3c4cccc5c4Oc4c3ccc3cccc(c43)-c3ccccc3N5)n2)cc1]

(1) Synthesis of Inter1-11-a

2-aminonaphthalene-1,8-diol (20.0 g, 114 mmol), 1,2-dibromo-3-chlorobenzene (30.86 g, 114 mmol), PPA (polyphosphoric acid) (400 mL) were added and stirred at 180° C. for 4 hours. When the reaction is complete, the mixture is extracted with CH₂Cl₂ and water, and the organic layer is dried over MgSO₄ and concentrated. Thereafter, the resulting organic material was recrystallized using silicagel column to obtain 17.5 g of a product. (Yield 54%)

(2) Synthesis of Inter1-11-b

After dissolving Inter1-11-a (17.5 g, 61.6 mmol) obtained in the above synthesis in toluene (310 mL) in a round-bottom flask, 2-chloro-4,6-diphenyl-1,3,5-triazine (16.5 g, 61.6 mmol), Pd₂(dba)₃ (1.69 g, 1.85 mmol), P(t-Bu)₃ (0.75 g, 3.70 mmol), NaOt-Bu (11.8 g, 123 mmol) were added and carried out at 50° C. When the reaction is complete, the mixture is extracted with CH₂Cl₂ and water, and the organic layer is dried over MgSO₄ and concentrated. Then, the resulting organic material was recrystallized by silicagel column to obtain 23.8 g of a product. (yield 75%)

(3) Synthesis of Inter1-11-c

Sub1-11-b (23.8 g, 46.2 mmol) obtained in the above synthesis was used for the synthesis of Inter2-1-b to obtain 23.6 g of a product. (yield 79%)

(4) Synthesis of Inter2-11

The obtained Inter1-11-c (23.6 g, 36.5 mmol), (2-aminophenyl)boronic acid (5.0 g, 36.5 mmol), K₂CO₃ (15.1 g, 109 mmol), Pd(PPh₃)₄ (2.53 g, 2.19 mmol), Pd(PPh₃)₄ (2.53 g, 2.19 mmol) were used for the synthesis of Inter2-1-a to obtain 15.3 g of a product. (Yield 71%)

(5) Synthesis of Sub1-11

The obtained Inter2-11 (15.3 g, 25.9 mmol), Pd₂(dba)₃ (0.71 g, 0.78 mmol), P(t-Bu)₃ (0.31 g, 1.56 mmol), NaOt-Bu (5.0 g, 51.9 mmol) were used for the synthesis of Sub1-1 to obtain 10.6 g of the product. (Yield 74%)

### 4. Synthesis Example of Sub1-17

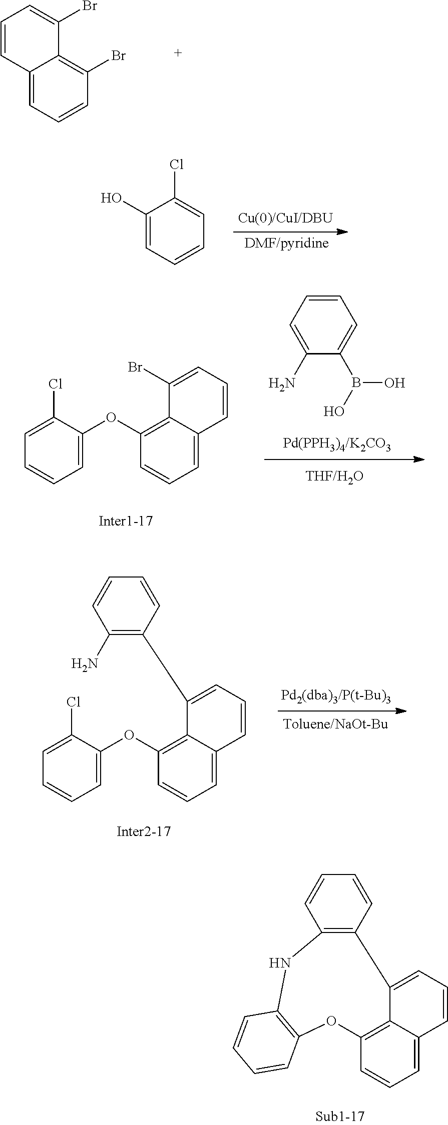 [SMILES: Brc1cccc2cccc(Br)c12 ; Oc1ccccc1Cl ; Clc1ccccc1Oc1cccc2cccc(Br)c12 ; Nc1ccccc1B(O)O ; Nc1ccccc1-c1cccc2cccc(Oc3ccccc3Cl)c12 ; c1ccc2c(c1)Nc1ccccc1-c1cccc3cccc(c13)O2]

(1) Synthesis of Inter1-17

1,8-dibromonaphthalene (15.0 g, 52.5 mmol), 2-chlorophenol (13.49 g, 105 mmol), 1,8-diazabicyclo[5.4.0]undec-7-ene (DBU) (24.0 g, 157 mmol), pyridine (0.85 mL), copper powder (0.43 g, 6.82 mmol), CuI (0.45 g, 2.36 mmol) were placed in a round flask, DMF (300 mL) was added and refluxed for 3 hours. When the reaction is complete, after cooling to room temperature, 3M HCl is added until the precipitation is complete. Thereafter, the precipitate was washed with water and dried to obtain 14.3 g of a product. (Yield 82%)

(2) Synthesis of Inter2-17

The obtained Inter1-17 (14.3 g, 43.0 mmol), (2-aminophenyl)boronic acid (5.9 g, 43.0 mmol), K₂CO₃ (17.8 g, 129 mmol), Pd(PPh₃)₄ (2.98 g, 2.58 mmol) were used for the synthesis of Inter2-1-a to obtain 11.0 g of the product. (Yield 74%)

(3) Synthesis of Sub1-17

The obtained Inter2-17 (11.0 g, 31.8 mmol), Pd₂(dba)₃ (0.87 g, 0.95 mmol), P(t-Bu)₃ (0.39 g, 1.91 mmol), NaOt-Bu (6.1 g, 63.6 mmol) were used for the synthesis of Sub1-1 to obtain 6.8 g of the product. (Yield 69%)

### 5. Synthesis Example of Sub1-65

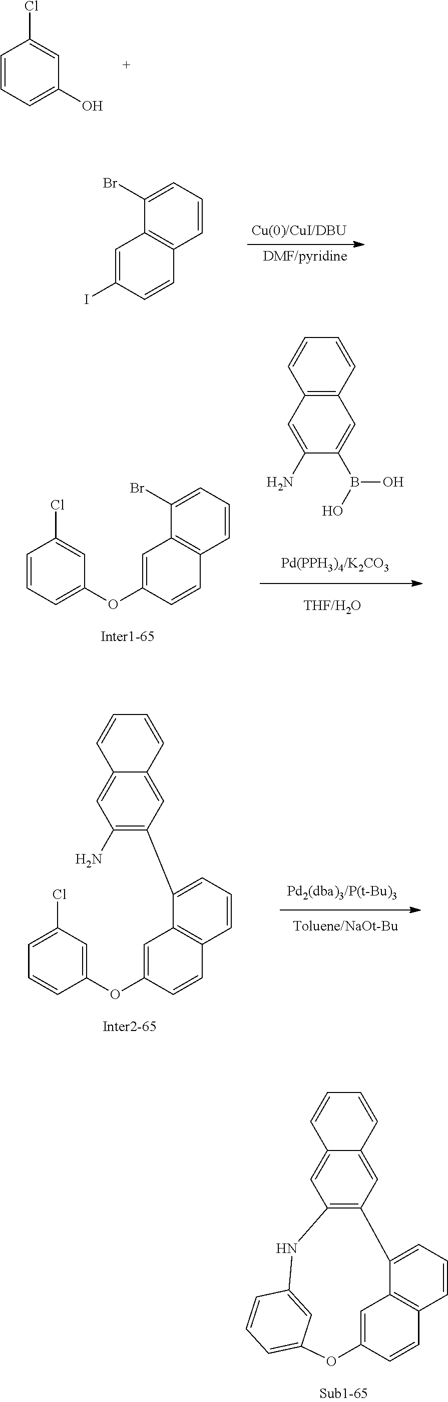 [SMILES: Oc1cccc(Cl)c1 ; Brc1cccc2ccc(I)cc12 ; Clc1cccc(Oc2ccc3cccc(Br)c3c2)c1 ; Nc1cc2ccccc2cc1B(O)O ; Nc1cc2ccccc2cc1-c1cccc2ccc(Oc3cccc(Cl)c3)cc12 ; c1cc2cc(c1)Oc1ccc3cccc(c3c1)-c1cc3ccccc3cc1N2]

(1) Synthesis of Inter1-65

1-bromo-7-iodonaphthalene (20.0 g, 60.1 mmol), 3-chlorophenol (15.4 g, 120 mmol), 1,8-diazabicyclo[5.4.0]undec-7-ene (DBU) (27.4 g, 180 mmol), pyridine (0.97 mL), copper powder (0.50 g, 7.81 mmol), CuI (0.51 g, 2.70 mmol) were used for the synthesis of Inter1-17 above to obtain 17.0 g of the product. (yield 85%)

(2) Synthesis of Inter2-65

The obtained Inter1-65 (17.0 g, 51.1 mmol), (3-aminonaphthalen-2-yl)boronic acid (9.5 g, 51.1 mmol), K₂CO₃ (21.2 g, 153 mmol), Pd(PPh₃)₄ (3.54 g, 3.06 mmol) were used for the synthesis of Inter2-1-a to obtain 14.1 g of the product. (Yield 70%)

(3) Synthesis of Sub1-65

The obtained Inter2-65 (14.1 g, 35.6 mmol), Pd₂(dba)₃ (0.98 g, 1.07 mmol), P(t-Bu)₃ (0.43 g, 2.14 mmol), NaOt-Bu (6.8 g, 71.2 mmol) were used for the synthesis of Sub1-1 to obtain 8.3 g of the product. (Yield 65%)

### 6. Synthesis Example of Sub1-78

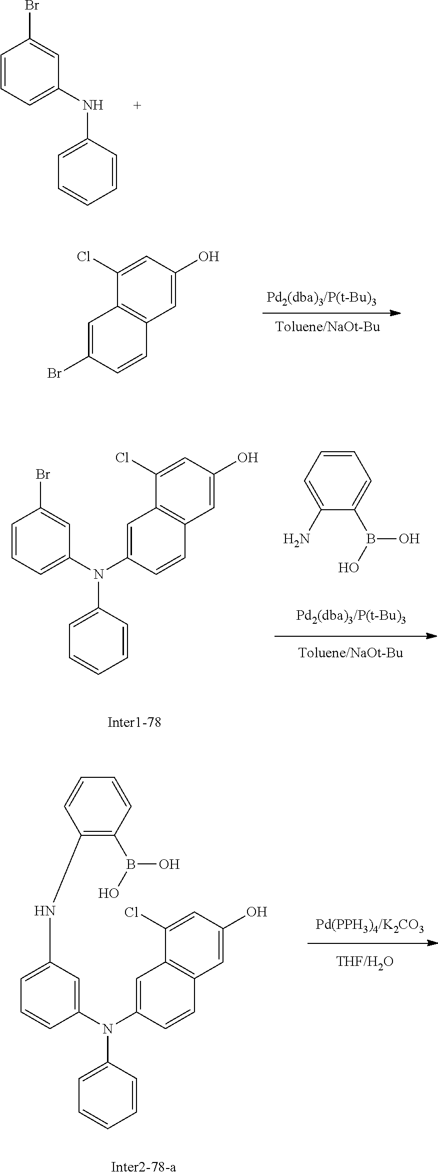 [SMILES: Brc1cccc(Nc2ccccc2)c1 ; Oc1cc(Cl)c2cc(Br)ccc2c1 ; Oc1cc(Cl)c2cc(N(c3ccccc3)c3cccc(Br)c3)ccc2c1 ; Nc1ccccc1B(O)O ; OB(O)c1ccccc1Nc1cccc(N(c2ccccc2)c2ccc3cc(O)cc(Cl)c3c2)c1]

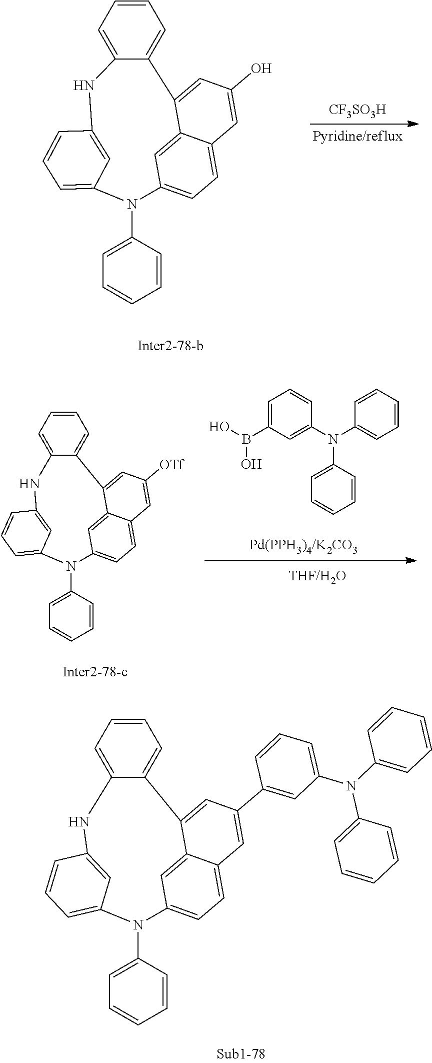

(1) Synthesis of Inter1-78

After dissolving 3-bromo-N-phenylaniline (20.0 g, 80.6 mmol) in toluene (400 mL) in a round-bottom flask, 6-bromo-4-chloronaphthalen-2-ol (20.8 g, 80.6 mmol), Pd₂(dba)₃ (2.21 g, 2.42 mmol), P(t-Bu)₃ (0.98 g, 4.84 mmol), NaOt-Bu (15.5 g, 161 mmol) were added and carried out at 60° C. When the reaction is complete, the mixture is extracted with CH₂Cl₂ and water, and the organic layer is dried over MgSO₄ and concentrated. Then, the resulting organic material was recrystallized by silicagel column to obtain 26.0 g of a product. (yield 76%)

(2) Synthesis of Inter2-78-a

After dissolving Inter1-78 (26.0 g, 61.3 mmol) obtained in the above synthesis in toluene (310 mL) in a round-bottom flask, (2-aminophenyl)boronic acid (8.4 g, 61.3 mmol), Pd₂(dba)₃ (1.68 g, 1.84 mmol), P(t-Bu)₃ (0.74 g, 3.68 mmol), NaOt-Bu (11.8 g, 123 mmol) were added and carried out at 60° C. When the reaction is complete, the mixture is extracted with CH₂Cl₂ and water, and the organic layer is dried over MgSO₄ and concentrated. Then, the resulting organic material was recrystallized by silicagel column to obtain 21.2 g of a product. (yield 72%)

(3) Synthesis of Inter2-78-b

The obtained Inter2-78-a (21.2 g, 44.1 mmol), K₂CO₃ (18.3 g, 132 mmol), Pd(PPh₃)₄ (3.06 g, 2.65 mmol), THE (440 mL) and water (220 mL) were used for the synthesis of Inter2-1-a to obtain 11.3 g of a product. (yield 64%)

(4) Synthesis of Inter2-78-c

The obtained Inter2-78-b (11.3 g, 28.2 mmol) was used for the above synthesis of Inter2-1-b to obtain 11.4 g of a product. (yield 76%)

(5) Synthesis of Sub1-78

The obtained Inter2-78-c (11.4 g, 21.4 mmol), (3-(diphenylamino)phenyl)boronic acid (6.2 g, 21.4 mmol), K₂CO₃ (8.9 g, 64.2 mmol), Pd(PPh₃)₄ (1.48 g, 1.28) mmol) were used for the of Inter2-1-a to obtain 10.9 g of a product. (yield 81%)

However, the compound belonging to Sub 1 may be a compound as follows, but is not limited thereto.

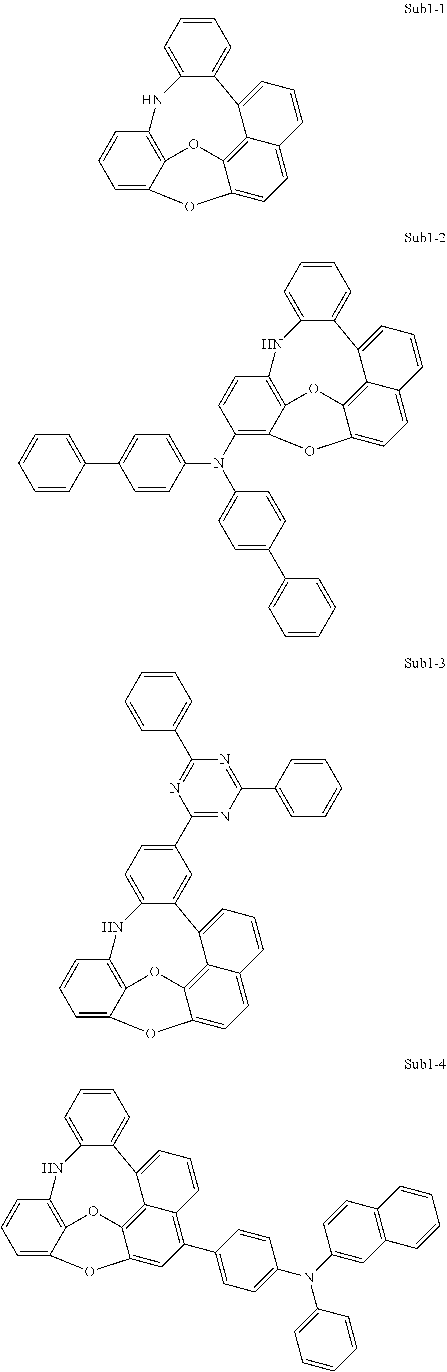 [SMILES: c1ccc2c(c1)Nc1cccc3c1Oc1c(ccc4cccc-2c14)O3 ; c1ccc(-c2ccc(N(c3ccc(-c4ccccc4)cc3)c3ccc4c5c3Oc3ccc6cccc(c6c3O5)-c3ccccc3N4)cc2)cc1 ; c1ccc(-c2nc(-c3ccccc3)nc(-c3ccc4c(c3)-c3cccc5ccc6c(c35)Oc3c(cccc3O6)N4)n2)cc1 ; c1ccc(N(c2ccc(-c3cc4c5c6c(cccc36)-c3ccccc3Nc3cccc(c3O5)O4)cc2)c2ccc3ccccc3c2)cc1]

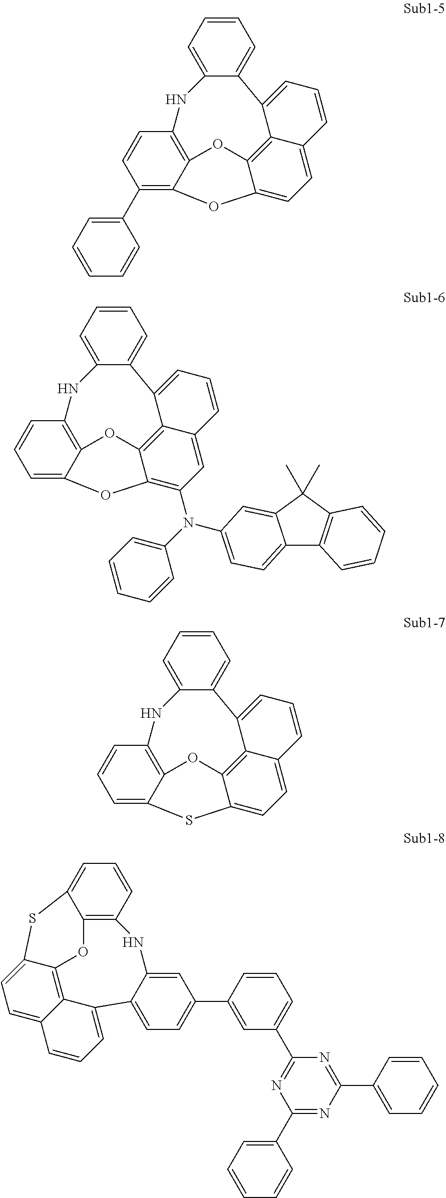

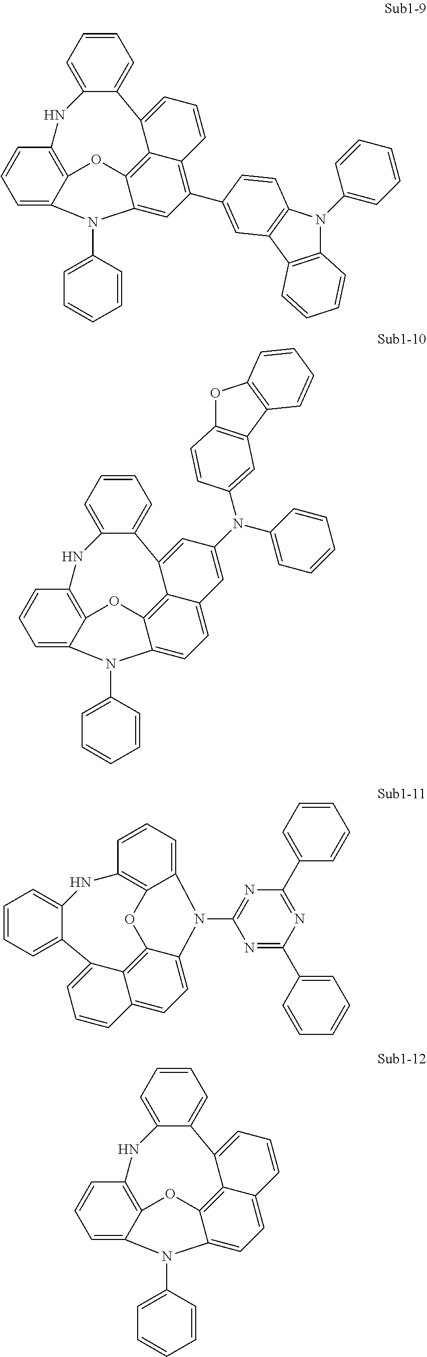

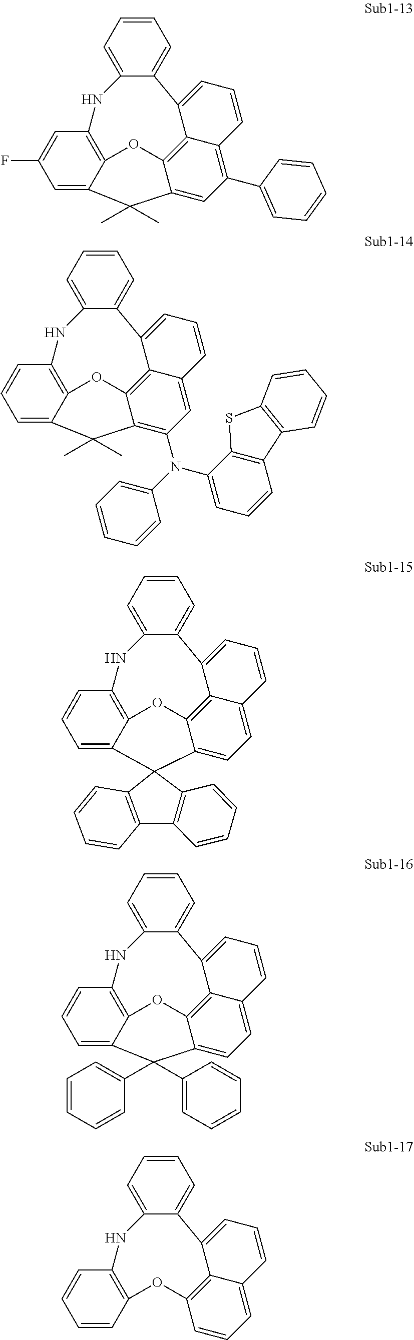

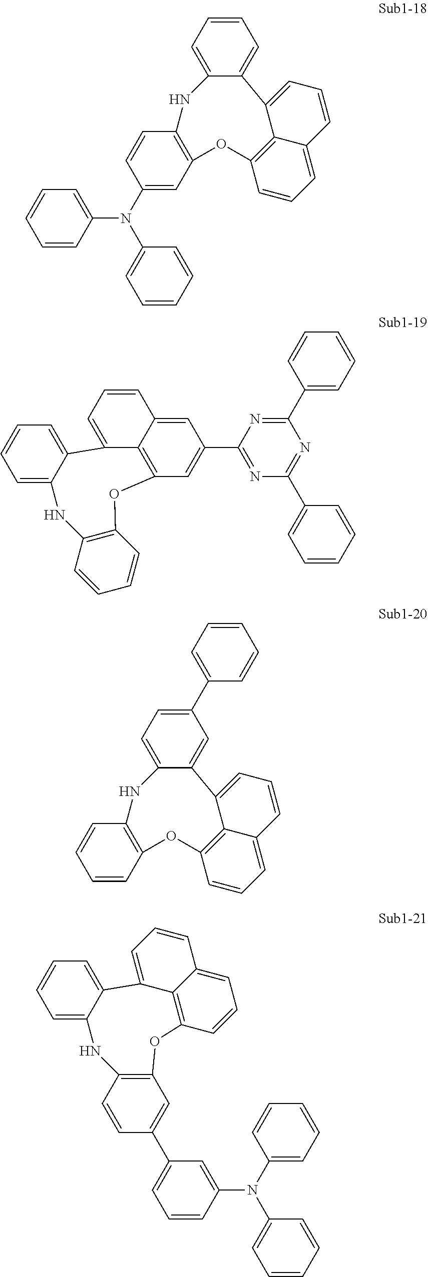 [SMILES: c1ccc(N(c2ccccc2)c2ccc3c(c2)Oc2cccc4cccc(c24)-c2ccccc2N3)cc1 ; c1ccc(-c2nc(-c3ccccc3)nc(-c3cc4c5c(cccc5c3)-c3ccccc3Nc3ccccc3O4)n2)cc1 ; c1ccc(-c2ccc3c(c2)-c2cccc4cccc(c24)Oc2ccccc2N3)cc1 ; c1ccc(N(c2ccccc2)c2cccc(-c3ccc4c(c3)Oc3cccc5cccc(c35)-c3ccccc3N4)c2)cc1]

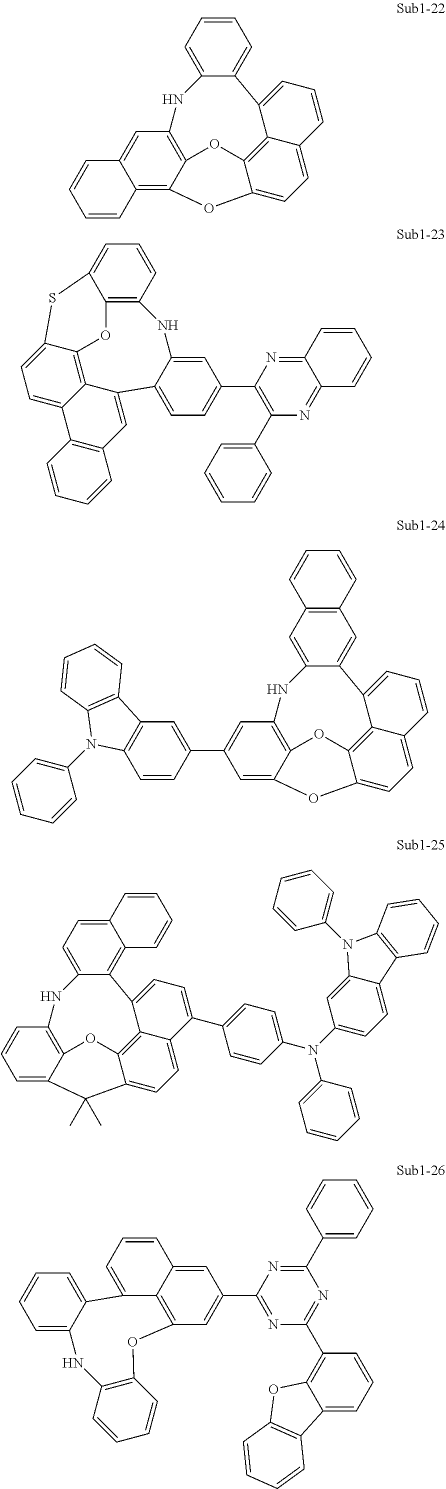

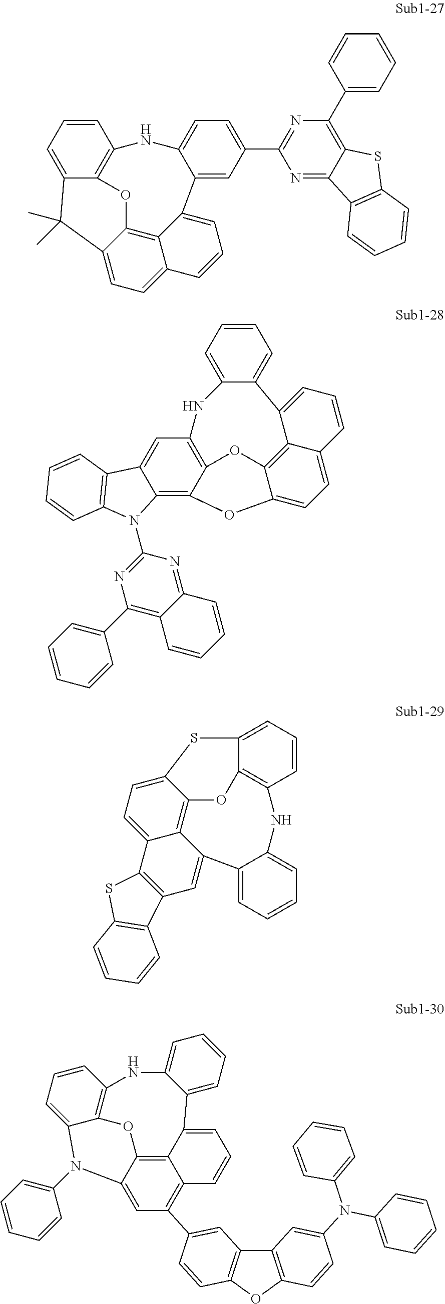 [SMILES: CC1(C)c2cccc3c2Oc2c1ccc1cccc(c21)-c1cc(-c2nc(-c4ccccc4)c4sc5ccccc5c4n2)ccc1N3 ; c1ccc(-c2nc(-n3c4ccccc4c4cc5c6c(c43)Oc3ccc4cccc(c4c3O6)-c3ccccc3N5)nc3ccccc23)cc1 ; c1ccc2c(c1)Nc1cccc3c1Oc1c(ccc4c1c-2cc1c2ccccc2sc41)S3 ; c1ccc(N(c2ccccc2)c2ccc3oc4ccc(-c5cc6c7c8c(cccc58)-c5ccccc5Nc5cccc(c5O7)N6c5ccccc5)cc4c3c2)cc1]

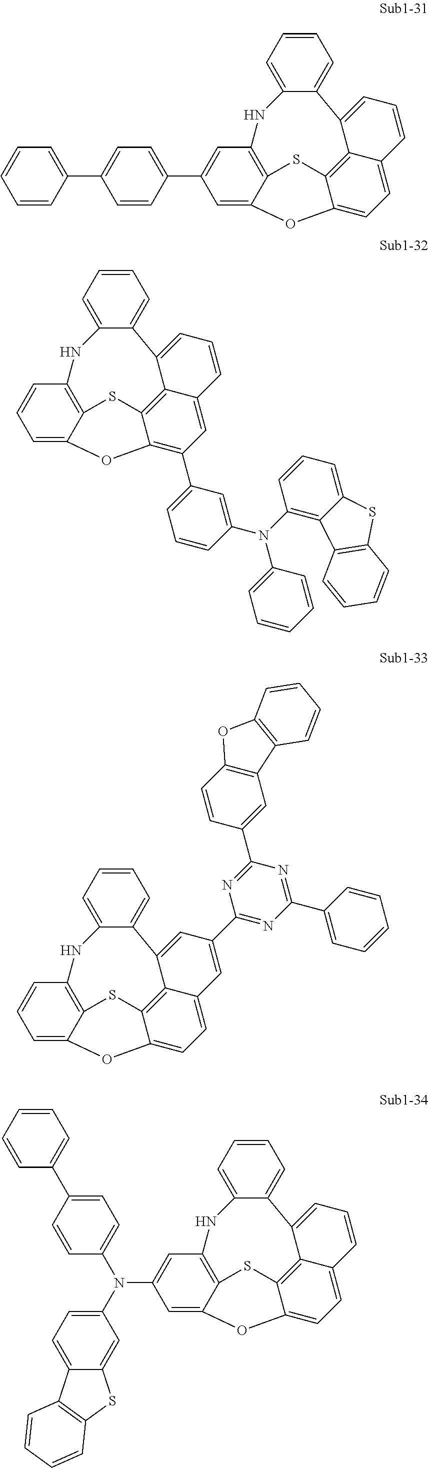 [SMILES: c1ccc(-c2ccc(-c3cc4c5c(c3)Oc3ccc6cccc(c6c3S5)-c3ccccc3N4)cc2)cc1 ; c1ccc(N(c2cccc(-c3cc4cccc5c4c4c3Oc3cccc(c3S4)Nc3ccccc3-5)c2)c2cccc3sc4ccccc4c23)cc1 ; c1ccc(-c2nc(-c3cc4c5c6c(ccc5c3)Oc3cccc(c3S6)Nc3ccccc3-4)nc(-c3ccc4oc5ccccc5c4c3)n2)cc1 ; c1ccc(-c2ccc(N(c3cc4c5c(c3)Oc3ccc6cccc(c6c3S5)-c3ccccc3N4)c3ccc4c(c3)sc3ccccc34)cc2)cc1]

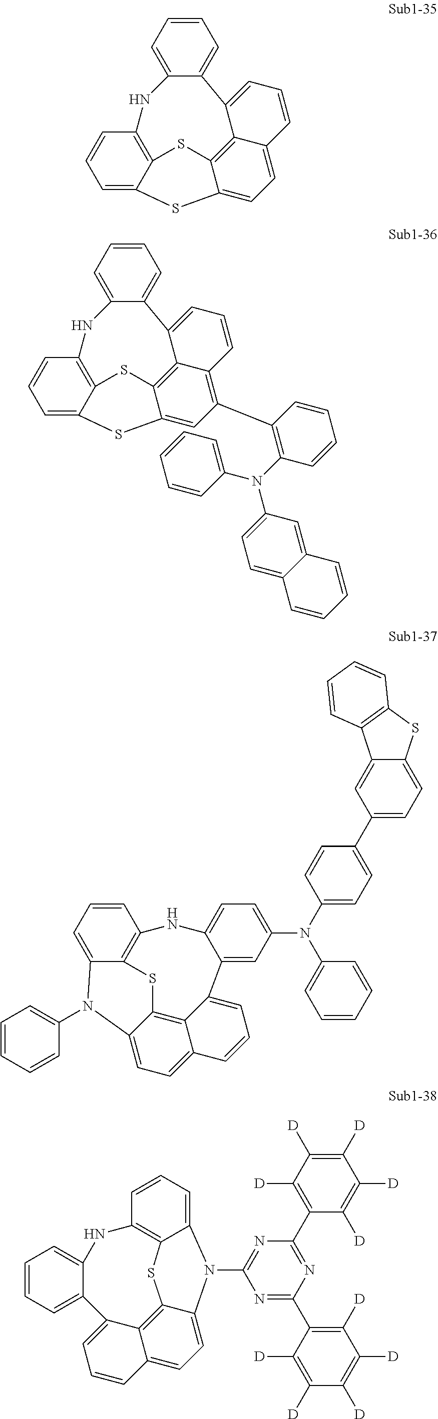 [SMILES: c1ccc2c(c1)Nc1cccc3c1Sc1c(ccc4cccc-2c14)S3 ; c1ccc(N(c2ccc3ccccc3c2)c2ccccc2-c2cc3c4c5c(cccc25)-c2ccccc2Nc2cccc(c2S4)S3)cc1 ; c1ccc(N(c2ccc(-c3ccc4sc5ccccc5c4c3)cc2)c2ccc3c(c2)-c2cccc4ccc5c(c24)Sc2c(cccc2N5c2ccccc2)N3)cc1 ; [2H]c1c([2H])c([2H])c(-c2nc(-c3c([2H])c([2H])c([2H])c([2H])c3[2H])nc(N3c4cccc5c4Sc4c3ccc3cccc(c43)-c3ccccc3N5)n2)c([2H])c1[2H]]

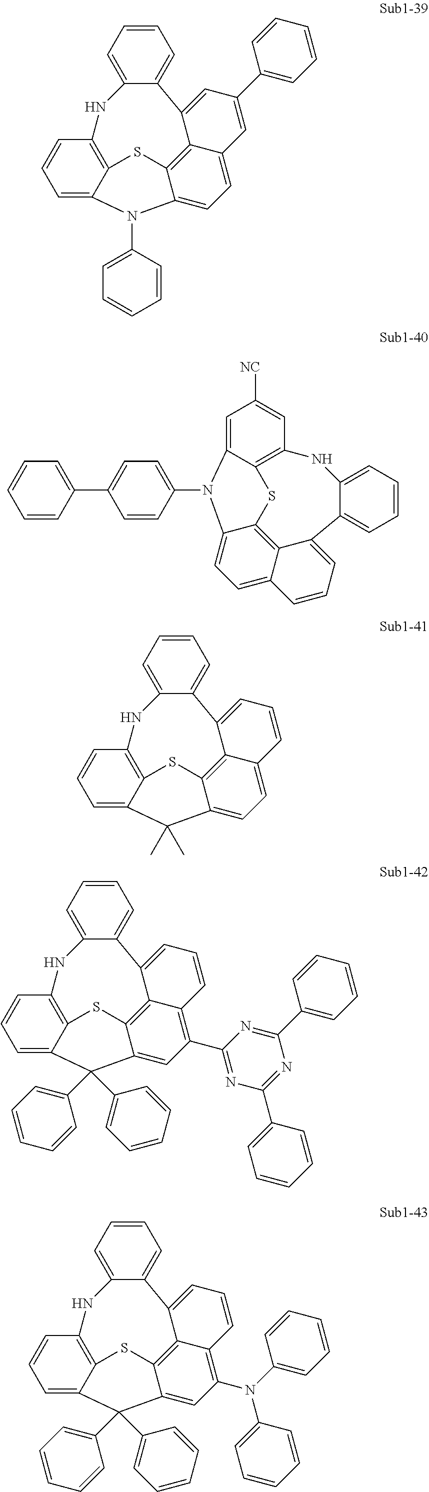

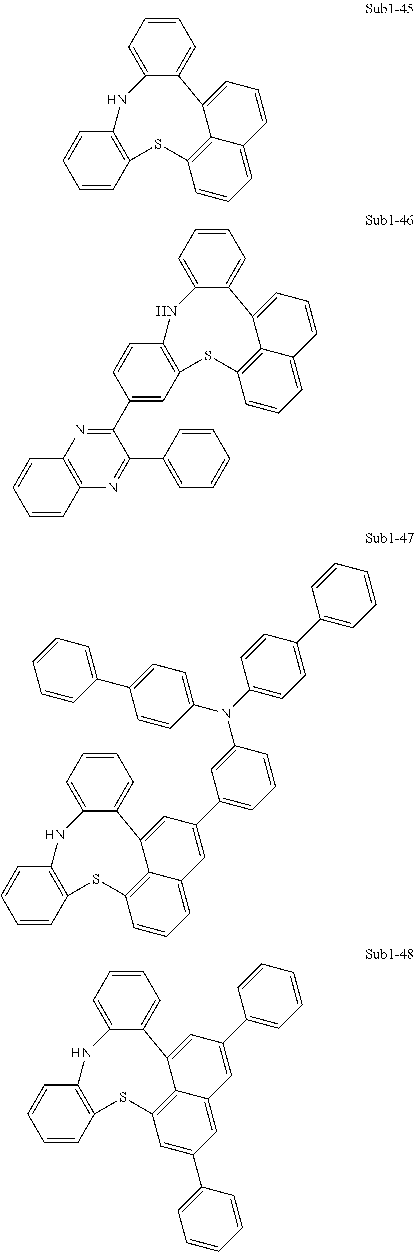

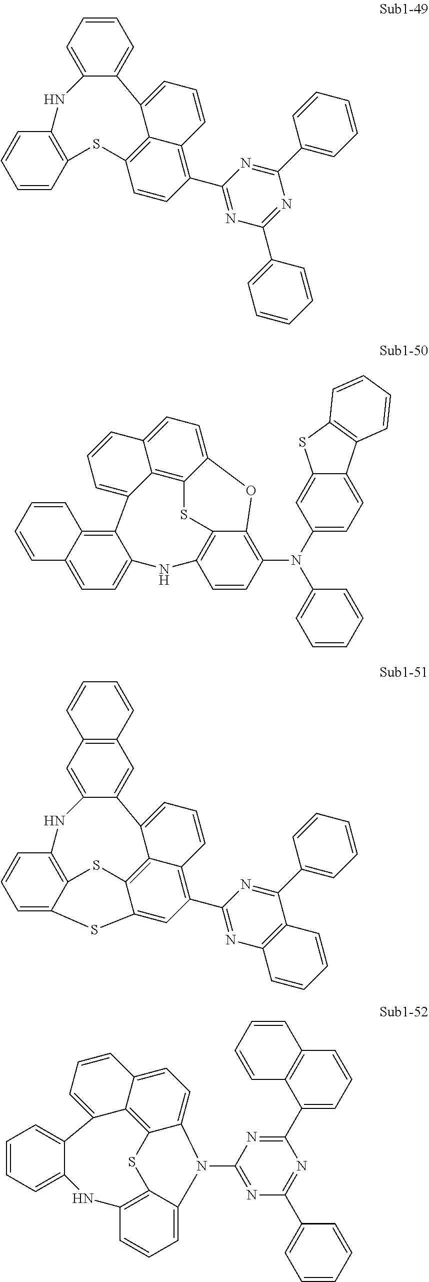

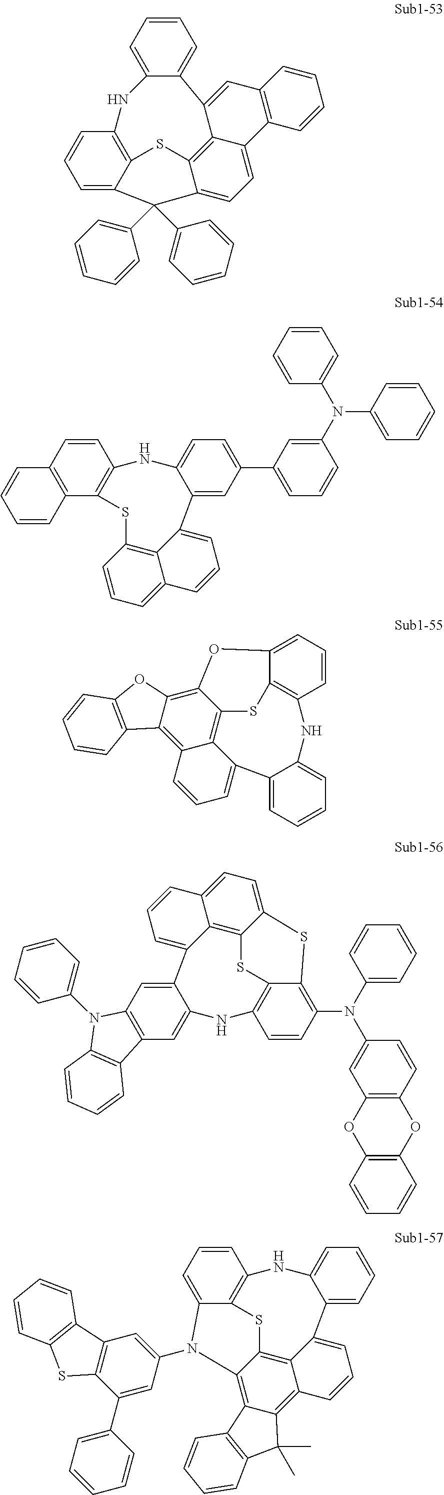

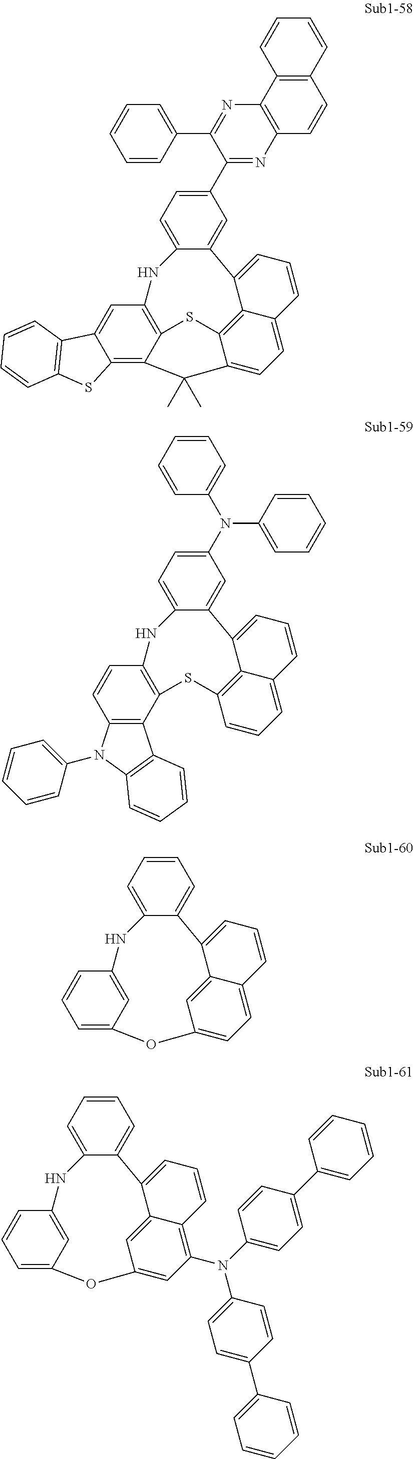

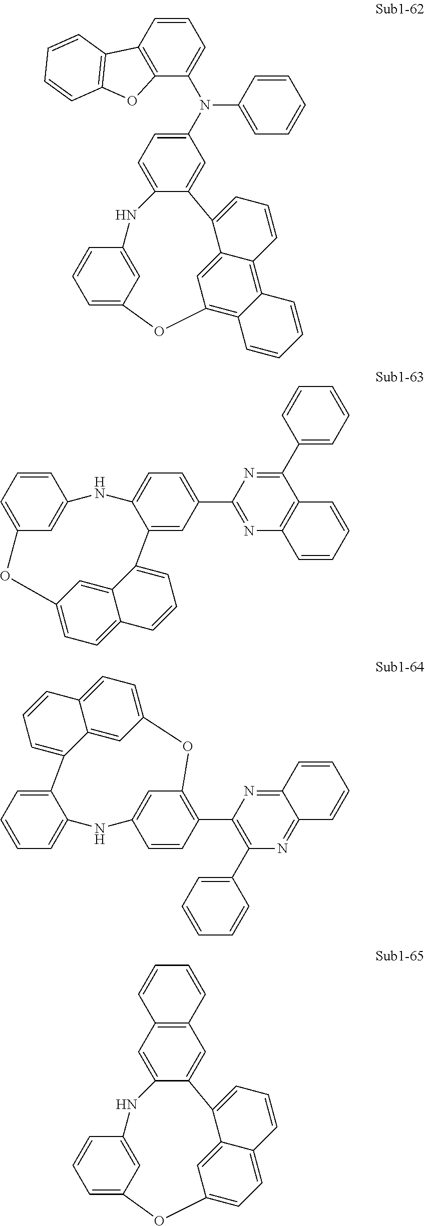

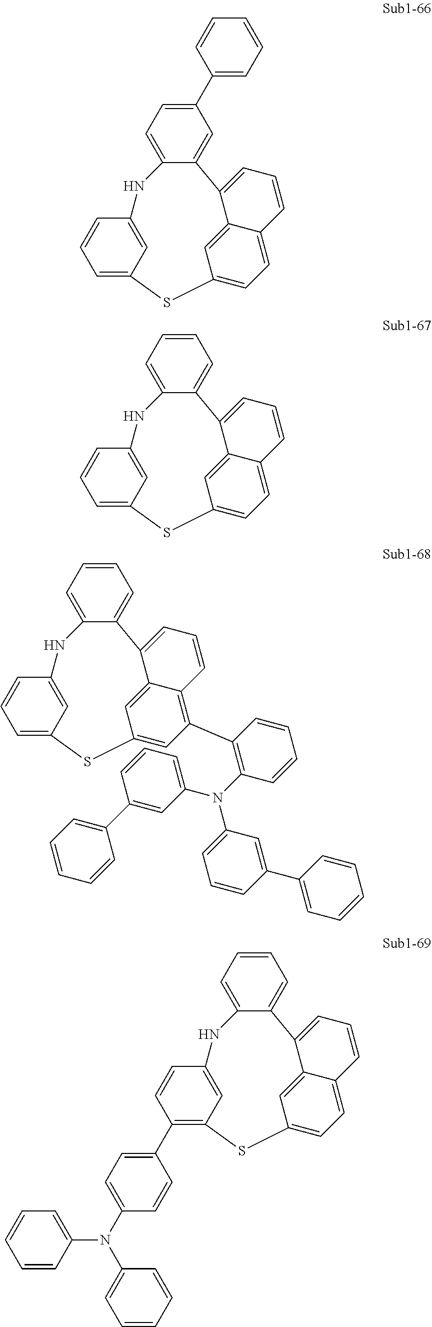

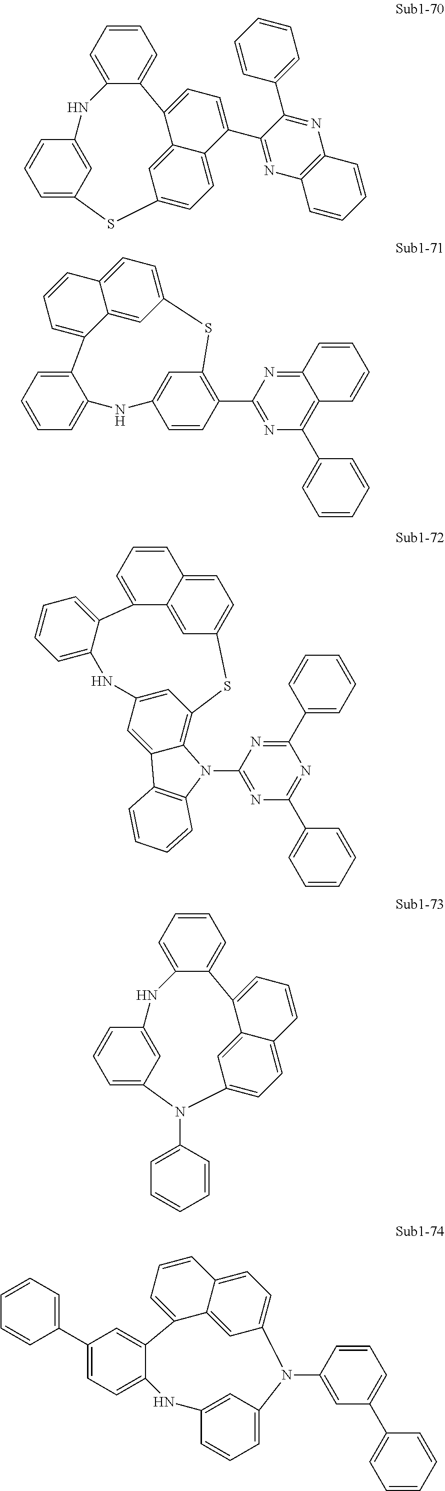

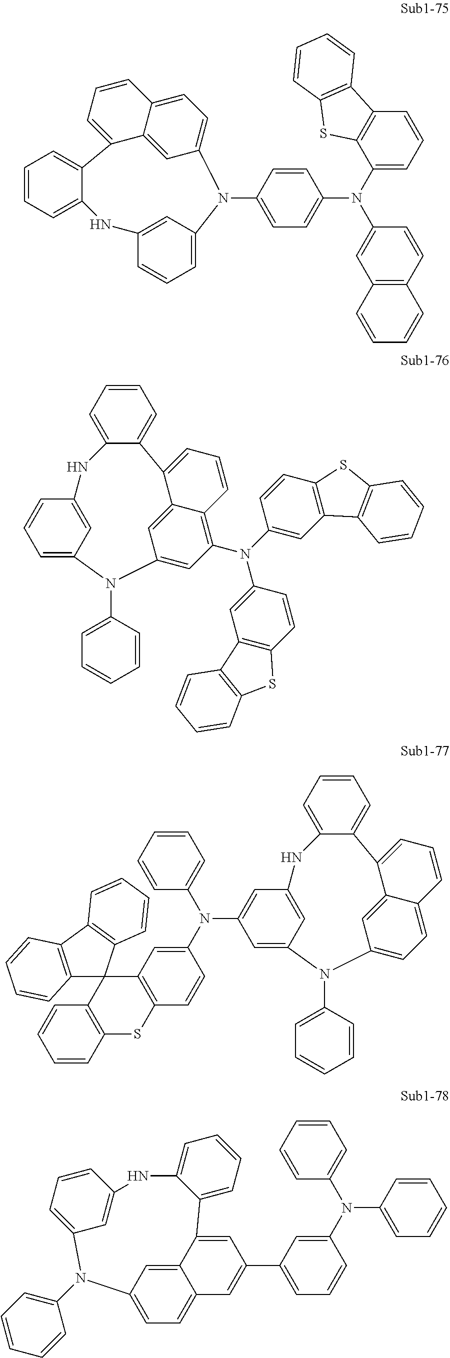 [SMILES: c1ccc2cc(N(c3ccc(-n4c5cccc(c5)[nH]c5ccccc5c5cccc6ccc4cc65)cc3)c3cccc4c3sc3ccccc34)ccc2c1 ; c1ccc(-n2c3cccc(c3)[nH]c3ccccc3c3cccc4c(N(c5ccc6sc7ccccc7c6c5)c5ccc6sc7ccccc7c6c5)cc2cc43)cc1 ; c1ccc(N(c2ccc3c(c2)C2(c4ccccc4S3)c3ccccc3-c3ccccc32)c2cc3cc(c2)n(-c2ccccc2)c2ccc4cccc(c4c2)c2ccccc2[nH]3)cc1 ; c1ccc(N(c2ccccc2)c2cccc(-c3cc4ccc5cc4c(c3)c3ccccc3[nH]c3cccc(c3)n5-c3ccccc3)c2)cc1]

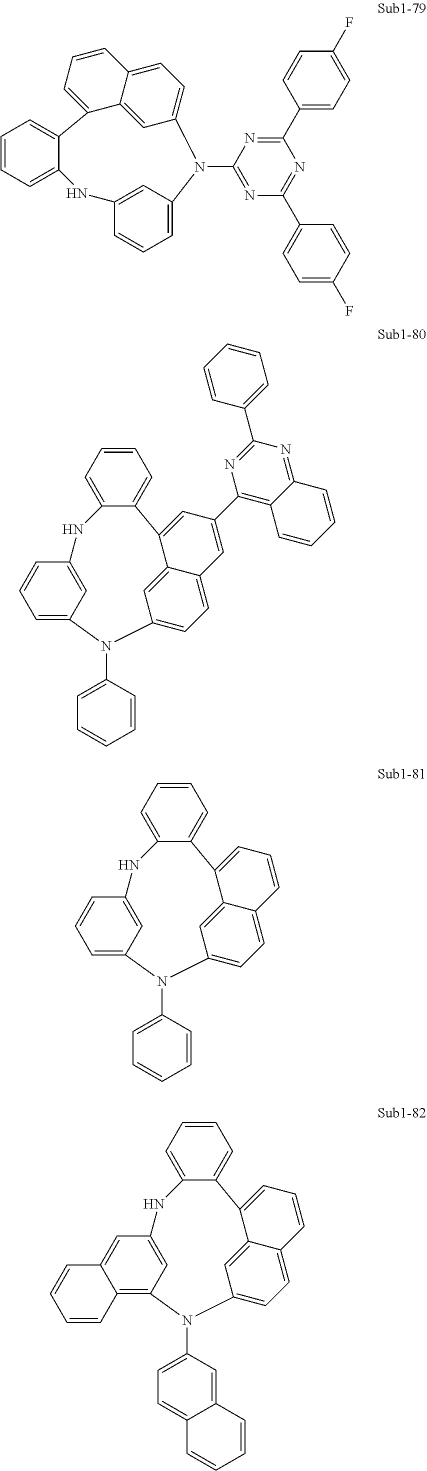

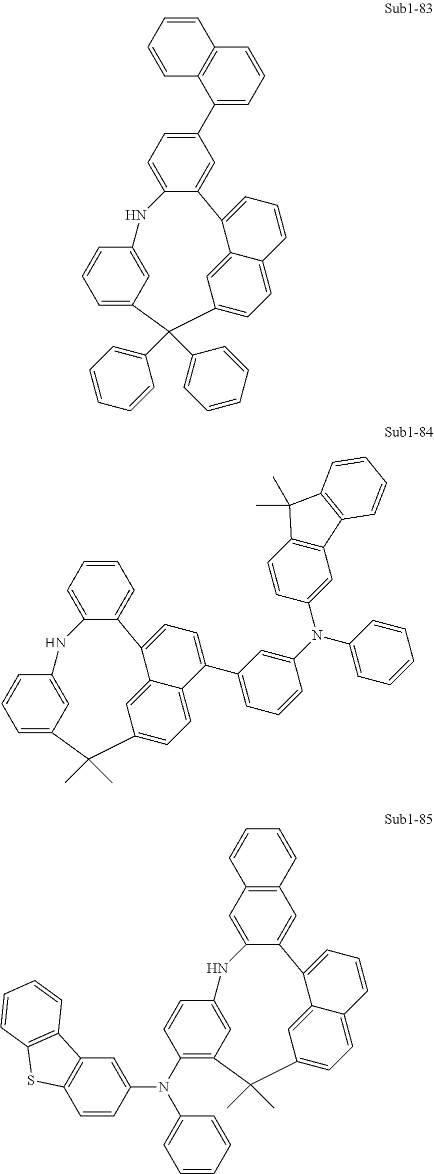 [SMILES: c1ccc(C2(c3ccccc3)c3cccc(c3)Nc3ccc(-c4cccc5ccccc45)cc3-c3cccc4ccc2cc34)cc1 ; CC1(C)c2cccc(c2)Nc2ccccc2-c2ccc(-c3cccc(N(c4ccccc4)c4ccc5c(c4)-c4ccccc4C5(C)C)c3)c3ccc1cc23 ; CC1(C)c2ccc3cccc(c3c2)-c2cc3ccccc3cc2Nc2ccc(N(c3ccccc3)c3ccc4sc5ccccc5c4c3)c1c2]

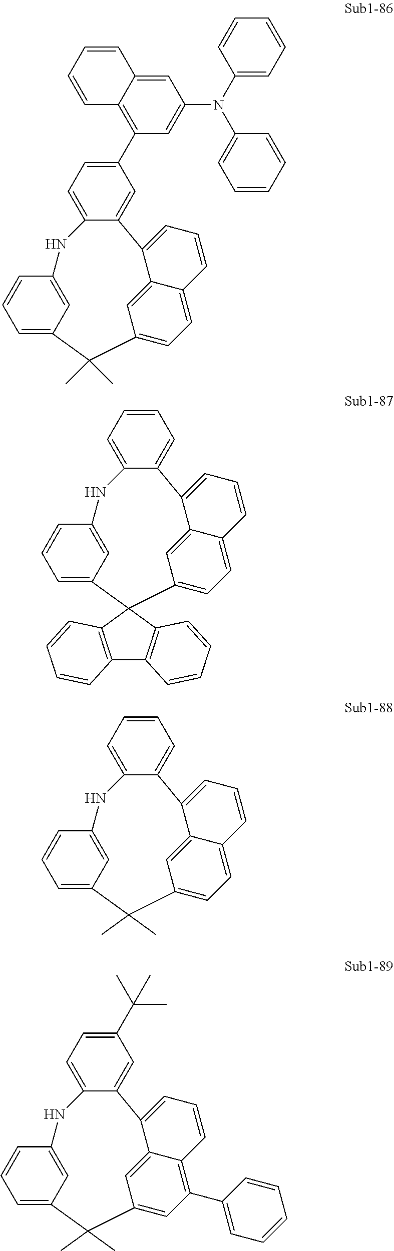

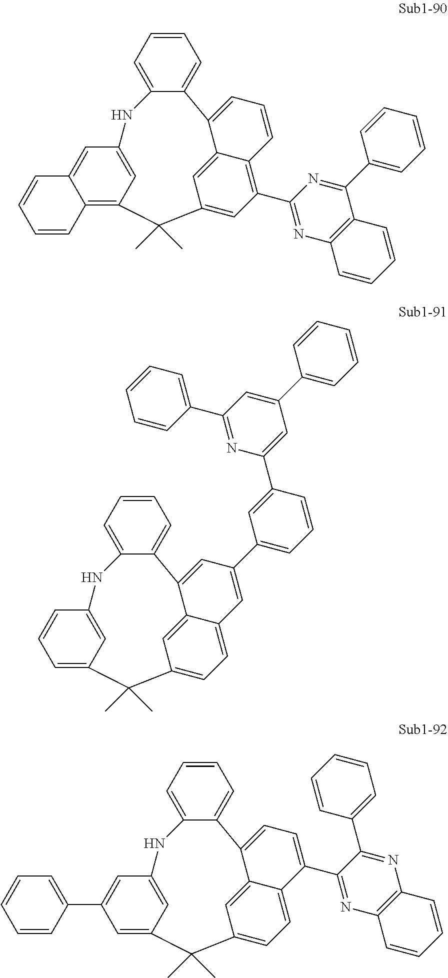 [SMILES: CC1(C)c2cc(-c3nc(-c4ccccc4)c4ccccc4n3)c3cccc(c3c2)-c2ccccc2Nc2cc1c1ccccc1c2 ; CC1(C)c2cccc(c2)Nc2ccccc2-c2cc(-c3cccc(-c4cc(-c5ccccc5)cc(-c5ccccc5)n4)c3)cc3ccc1cc23 ; CC1(C)c2cc(cc(-c3ccccc3)c2)Nc2ccccc2-c2ccc(-c3nc4ccccc4nc3-c3ccccc3)c3ccc1cc23]

Table 1 below shows the FD-MS (Field Desorption-Mass Spectrometry) values of the compounds belonging to Sub 1.

[Table 1

| compound | FD-MS | compound | FD-MS |
|---|---|---|---|
| Sub1-1 | m/z = 323.09(C₂₂H₁₃NO₂ = 323.35) | Sub1-2 | m/z = 642.23(C₄₆H₃₀N₂O₂ = 642.76) |
| Sub1-3 | m/z = 554.17(C₃₇H₂₂N₄O₂ = 554.61) | Sub1-4 | m/z = 616.22(C₄₄H₂₈N₂O₂ = 616.72) |
| Sub1-5 | m/z = 415.1(C₂₈H₁₇NOS = 415.51) | Sub1-6 | m/z = 622.21(C₄₃H₃₀N₂OS = 622.79) |
| Sub1-7 | m/z = 339.07(C₂₂H₁₃NOS = 339.41) | Sub1-8 | m/z = 646.18(C₄₃H₂₆N₄OS = 646.77) |
| Sub1-9 | m/z = 639.23(C₄₆H₂₉N₃O = 639.76) | Sub1-10 | m/z = 655.23(C₄₆H₂₉N₃O₂ = 655.76) |
| Sub1-11 | m/z = 553.19(C₃₇H₂₃N₅O = 553.63) | Sub1-12 | m/z = 398.14(C₂₈H₁₈N₂O = 398.47) |
| Sub1-13 | m/z = 443.17(C₃₁H₂₂FNO = 443.52) | Sub1-14 | m/z = 622.21(C₄₃H₃₀N₂OS = 622.79) |
| Sub1-15 | m/z = 471.16(C₃₅H₂₁NO = 471.56) | Sub1-16 | m/z = 473.18(C₃₅H₂₃NO = 473.58) |
| Sub1-17 | m/z = 309.12(C₂₂H₁₅NO = 309.37) | Sub1-18 | m/z = 476.19(C₃₄H₂₄N₂O = 476.58) |
| Sub1-19 | m/z = 540.2(C₃₇H₂₄N₄O = 540.63) | Sub1-20 | m/z = 385.15(C₂₈H₁₉NO = 385.47) |
| Sub1-21 | m/z = 552.22(C₄₀H₂₈N₂O = 552.68) | Sub1-22 | m/z = 373.11(C₂₆H₁₅NO₂ = 373.41) |
| Sub1-23 | m/z = 593.16(C₄₀H₂₃N₃OS = 593.7) | Sub1-24 | m/z = 689.25(C₅₀H₃₁N₃O = 689.82) |
| Sub1-25 | m/z = 807.32(C₅₉H₄₁N₃O = 808) | Sub1-26 | m/z = 680.22(C₄₇H₂₈N₄O₂ = 680.77) |
| Sub1-27 | m/z = 609.19(C₄₁H₂₇N₃OS = 609.75) | Sub1-28 | m/z = 616.19(C₄₂H₂₄N₄O₂ = 616.68) |
| Sub1-29 | m/z = 445.06(C₂₈H₁₅NOS₂ = 445.55) | Sub1-30 | m/z = 821.27(C₅₈H₃₅N₃O₃ = 821.94) |
| Sub1-31 | m/z = 491.13(C₃₄H₂₁NOS = 491.61) | Sub1-32 | m/z = 688.16(C₄₆H₂₈N₂OS₂ = 688.86) |
| Sub1-33 | m/z = 660.16(C₄₃H₂₄N₄O₂S = 660.75) | Sub1-34 | m/z = 688.16(C₄₆H₂₈N₂OS₂ = 688.86) |
| Sub1-35 | m/z = 355.05(C₂₂H₁₃NS₂ = 355.47) | Sub1-36 | m/z = 648.17(C₄₄H₂₈N₂S₂ = 648.84) |
| Sub1-37 | m/z = 763.21(C₅₂H₃₃N₃S₂ = 763.98) | Sub1-38 | m/z = 579.23(C₃₇H₁₃D₁₀N₅S = 579.75) |
| Sub1-39 | m/z = 490.15(C₃₄H₂₂N₂S = 490.62) | Sub1-40 | m/z = 515.15(C₃₅H₂₁N₃S = 515.63) |
| Sub1-41 | m/z = 365.12(C₂₅H₁₉NS = 365.49) | Sub1-42 | m/z = 720.23(C₅₀H₃₂N₄S = 720.89) |
| Sub1-43 | m/z = 532.2(C₃₇H₂₈N₂S = 532.71) | Sub1-44 | m/z = 487.14(C₃₅H₂₁NS = 487.62) |
| Sub1-45 | m/z = 325.09(C₂₂H₁₅NS = 325.43) | Sub1-46 | m/z = 529.16(C₃₆H₂₃N₃S = 529.66) |
| Sub1-47 | m/z = 720.26(C₅₂H₃₆N₂S = 720.93) | Sub1-48 | m/z = 477.16(C₃₄H₂₃NS = 477.63) |
| Sub1-49 | m/z = 556.17(C₃₇H₂₄N₄S = 556.69) | Sub1-50 | m/z = 662.15(C₄₄H₂₆N₂OS₂ = 662.83) |
| Sub1-51 | m/z = 609.13(C₄₀H₂₃N₃S₂ = 609.77) | Sub1-52 | m/z = 669.2(C₄₅H₂₇N₅S = 669.81) |
| Sub1-53 | m/z = 539.17(C₃₉H₂₅NS = 539.7) | Sub1-54 | m/z = 618.21(C₄₄H₃₀N₂S = 618.8) |
| Sub1-55 | m/z = 429.08(C₂₈H₁₅NO₂S = 429.49) | Sub1-56 | m/z = 793.19(C₅₂H₃₁N₃O₂S ₂ = 793.96) |
| Sub1-57 | m/z = 714.19(C₄₇H₃₀N₄S₂ = 714.91) | Sub1-58 | m/z = 725.2(C₄₉H₃₁N₃S₂ = 725.93) |
| Sub1-59 | m/z = 657.22(C₄₆H₃₁N₃S = 657.84) | Sub1-60 | m/z = 309.12(C₂₂H₁₅NO = 309.37) |
| Sub1-61 | m/z = 628.25(C₄₆H₃₂N₂O = 628.78) | Sub1-62 | m/z = 616.22(C₄₄H₂₈N₂O₂ = 616.72) |
| Sub1-63 | m/z = 513.18(C₃₆H₂₃N₃O = 513.6) | Sub1-64 | m/z = 513.18(C₃₆H₂₃N₃O = 513.6) |
| Sub1-65 | m/z = 359.13(C₂₆H₁₇NO = 359.43) | Sub1-66 | m/z = 401.12(C₂₈H₁₉NS = 401.53) |
| Sub1-67 | m/z = 325.09(C₂₂H₁₅NS = 325.43) | Sub1-68 | m/z = 720.26(C₅₂H₃₆N₂S = 720.93) |
| Sub1-69 | m/z = 568.2(C₄₀H₂₈N₂S = 568.74) | Sub1-70 | m/z = 529.16(C₃₆H₂₃N₃S = 529.66) |
| Sub1-71 | m/z = 529.16(C₃₆H₂₃N₃S = 529.66) | Sub1-72 | m/z = 645.2(C₄₃H₂₇N₅S = 645.78) |
| Sub1-73 | m/z = 460.19(C₃₄H₂₄N₂ = 460.58) | Sub1-74 | m/z = 536.23(C₄₀H₂₈N₂ = 536.68) |
| Sub1-75 | m/z = 707.24(C₅₀H₃₃N₃S = 707.9) | Sub1-76 | m/z = 763.21(C₅₂H₃₃N₃S₂ = 763.98) |
| Sub1-77 | m/z = 821.29(C₅₉H₃₉N₃S = 822.04) | Sub1-78 | m/z = 627.27(C₄₆H₃₃N₃ = 627.79) |
| Sub1-79 | m/z = 575.19(C₃₇H₂₃F₂N₅ = 575.62) | Sub1-80 | m/z = 588.23(C₄₂H₂₈N₄ = 588.71) |
| Sub1-81 | m/z = 384.16(C₂₈H₂₀N₂ = 384.48) | Sub1-82 | m/z = 484.19(C₃₆H₂₄N₂ = 484.6) |
| Sub1-83 | m/z = 585.25(C₄₅H₃₁N = 585.75) | Sub1-84 | m/z = 694.33(C₅₂H₄₂N₂ = 694.92) |
| Sub1-85 | m/z = 658.24(C₄₇H₃₄N₂S = 658.86) | Sub1-86 | m/z = 628.29(C₄₇H₃₆N₂ = 628.82) |
| Sub1-87 | m/z = 457.18(C₃₅H₂₃N = 457.58) | Sub1-88 | m/z = 335.17(C₂₅H₂₁N = 335.45) |
| Sub1-89 | m/z = 467.26(C₃₅H₃₃N = 467.66) | Sub1-90 | m/z = 589.25(C₄₃H₃₁N₃ = 589.74) |
| Sub1-91 | m/z = 640.29(C₄₈H₃₆N₂ = 640.83) | Sub1-92 | m/z = 615.27(C₄₅H₃₃N₃ = 615.78) |

II. Synthesis of Sub 2

The compound belonging to Sub 2 may be a compound as follows, but is not limited thereto.

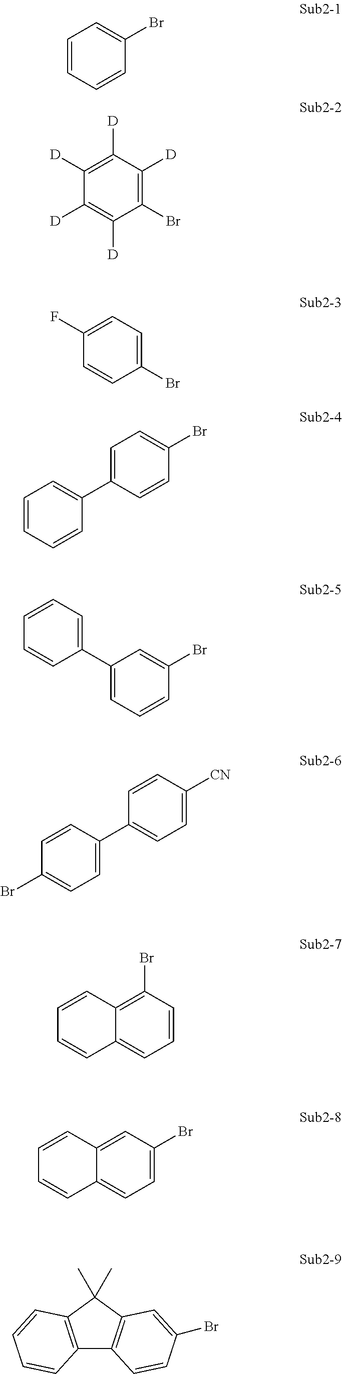

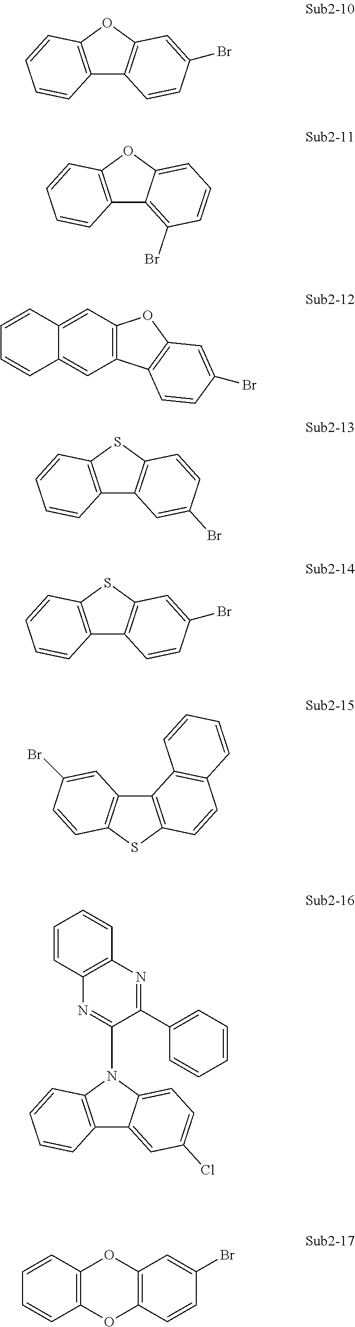

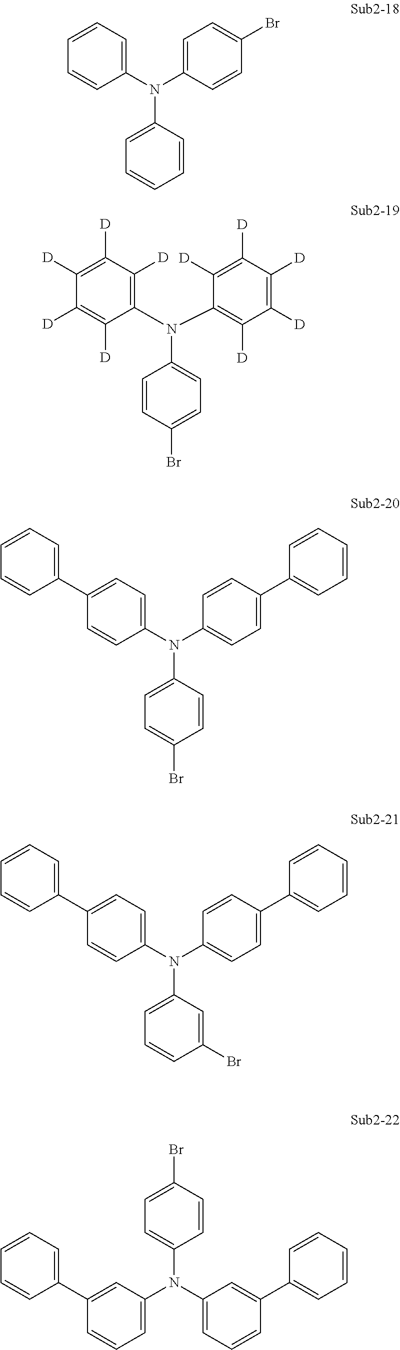 [SMILES: Brc1ccc(N(c2ccccc2)c2ccccc2)cc1 ; [2H]c1c([2H])c([2H])c(N(c2ccc(Br)cc2)c2c([2H])c([2H])c([2H])c([2H])c2[2H])c([2H])c1[2H] ; Brc1ccc(N(c2ccc(-c3ccccc3)cc2)c2ccc(-c3ccccc3)cc2)cc1 ; Brc1cccc(N(c2ccc(-c3ccccc3)cc2)c2ccc(-c3ccccc3)cc2)c1 ; Brc1ccc(N(c2cccc(-c3ccccc3)c2)c2cccc(-c3ccccc3)c2)cc1]

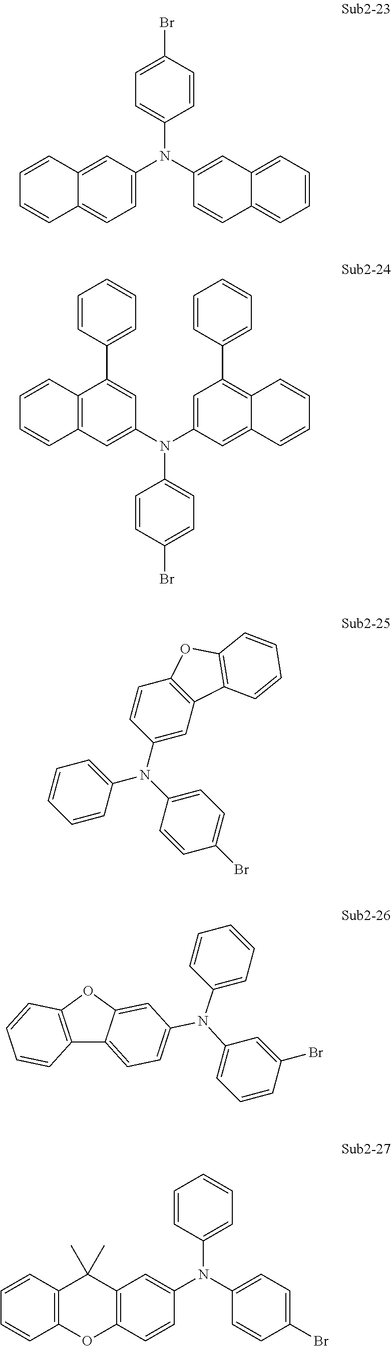 [SMILES: Brc1ccc(N(c2ccc3ccccc3c2)c2ccc3ccccc3c2)cc1 ; Brc1ccc(N(c2cc(-c3ccccc3)c3ccccc3c2)c2cc(-c3ccccc3)c3ccccc3c2)cc1 ; Brc1ccc(N(c2ccccc2)c2ccc3oc4ccccc4c3c2)cc1 ; Brc1cccc(N(c2ccccc2)c2ccc3c(c2)oc2ccccc23)c1 ; CC1(C)c2ccccc2Oc2ccc(N(c3ccccc3)c3ccc(Br)cc3)cc21]

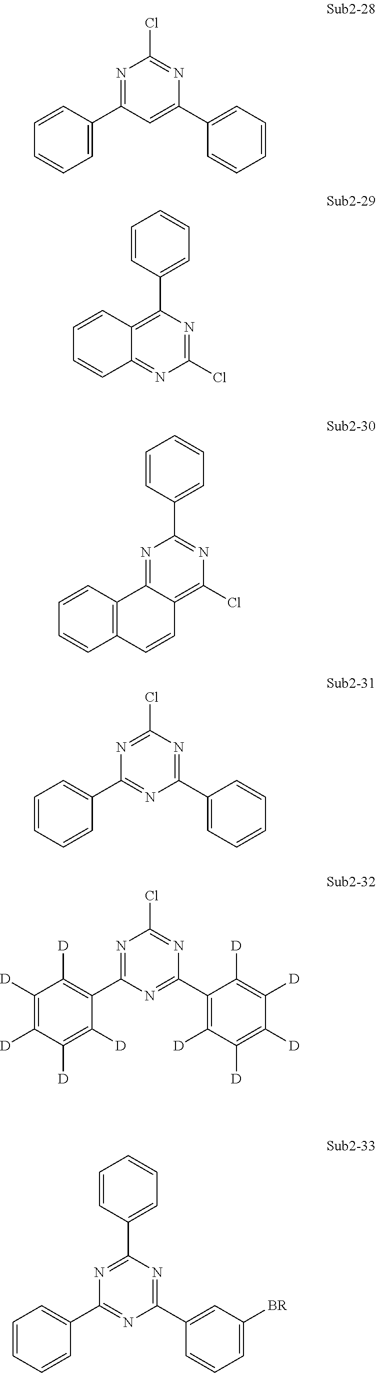

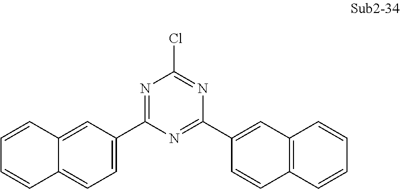 [SMILES: Clc1nc(-c2ccc3ccccc3c2)nc(-c2ccc3ccccc3c2)n1]

Table 2 below shows ED-MS (Field Desorption-Mass Spectrometry) values of compounds belonging to Sub 2 above.

TABLE 2

| compound | FD-MS | compound | FD-MS |
|---|---|---|---|
| Sub2-1 | m/z = 155.96(C₆H₅Br = 157.01) | Sub2-2 | m/z = 160.99(C₆D₅Br = 162.04) |
| Sub2-3 | m/z = 173.95(C₆H₄BrF = 175) | Sub2-4 | m/z = 231.99(C₁₂H₉Br = 233.11) |
| Sub2-5 | m/z = 231.99(C₁₂H₉Br = 233.11) | Sub2-6 | m/z = 256.98(C₁₃H₈BrN = 258.12) |
| Sub2-7 | m/z = 205.97(C₁₀H₇Br = 207.07) | Sub2-8 | m/z = 205.97(C₁₀H₇Br = 207.07) |
| Sub2-9 | m/z = 272.02(C₁₅H₁₃Br = 273.17) | Sub2-10 | m/z = 245.97(C₁₂H₇BrO = 247.09) |
| Sub2-11 | m/z = 245.97(C₁₂H₇BrO = 247.09) | Sub2-12 | m/z = 295.98(C₁₆H₉BrO = 297.15) |
| Sub2-13 | m/z = 261.95(C₁₂H₇BrS = 263.15) | Sub2-14 | m/z = 261.95(C₁₂H₇BrS = 263.15) |
| Sub2-15 | m/z = 311.96(C₁₆H₉BrS = 313.21) | Sub2-16 | m/z = 405.1(C₂₆H₁₆CIN₃ = 405.89) |
| Sub2-17 | m/z = 261.96(C₁₂H₇BrO₂ = 263.09) | Sub2-18 | m/z = 323.03(C₁₈H₁₄BrN = 324.22) |
| Sub2-19 | m/z = 333.09(C₁₈H₄D₁₀BrN = 334.28) | Sub2-20 | m/z = 475.09(C₃₀H₂₂BrN = 476.42) |
| Sub2-21 | m/z = 475.09(C₃₀H₂₂BrN = 476.42) | Sub2-22 | m/z = 475.09(C₃₀H₂₂BrN = 476.42) |
| Sub2-23 | m/z = 423.06(C₂₆H₁₈BrN = 424.34) | Sub2-24 | m/z = 575.12(C₃₈H₂₆BrN = 576.54) |
| Sub2-25 | m/z = 413.04(C₂₄H₁₆BrNO = 414.3) | Sub2-26 | m/z = 413.04(C₂₄H₁₆BrNO = 414.3) |
| Sub2-27 | m/z = 455.09(C₂₇H₂₂BrNO = 456.38) | Sub2-28 | m/z = 266.06(C₁₆H₁₁CIN₂ = 266.73) |
| Sub2-29 | m/z = 240.05(C₁₄H₉CIN₂ = 240.69) | Sub2-30 | m/z = 290.06(C₁₈H₁₁CIN₂ = 290.75) |
| Sub2-31 | m/z = 267.06(C₁₅H₁₀CIN₃ = 267.72) | Sub2-32 | m/z = 277.12(C₁₅D₁₀CIN₃ = 277.78) |
| Sub2-33 | m/z = 387.04(C₂₁H₁₄BrN₃ = 388.27) | Sub2-34 | m/z = 367.09(C₂₃H₁₄CIN₃ = 367.84) |

III. Synthesis of Final Product

### 1. Synthesis Example of P-1

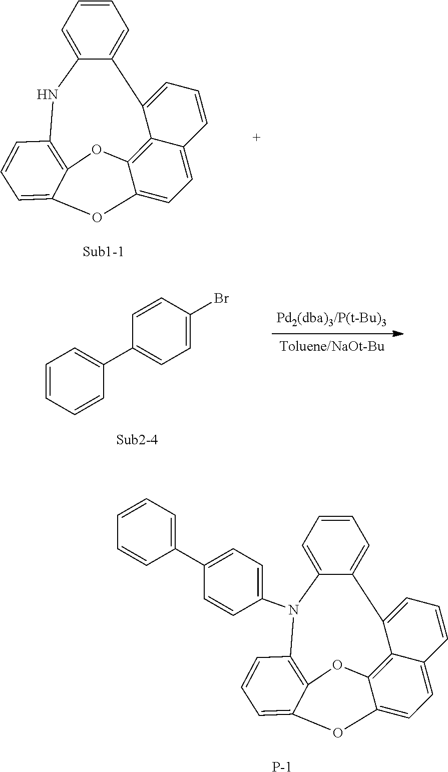

The obtained Sub 1-1 (5.0 g, 15.5 mmol) was placed in a round-bottom flask and dissolved with Toluene (77 mL), and Sub 2-4 (3.6 g, 15.5 mmol), Pd₂(dba)₃ (0.42 g, 0.46 mmol), P(t-Bu)₃ (0.19 g, 0.93 mmol), NaOt-Bu (3.0 g, 30.9 mmol) were added and stirred at 120° C. When the reaction is complete, the mixture is extracted with CH₂Cl₂ and water, and the organic layer is dried over MgSO₄ and concentrated. Then, the resulting organic material was recrystallized by silicagel column to obtain 5.7 g of a product. (yield 77%)

### 2. Synthesis Example of P-2

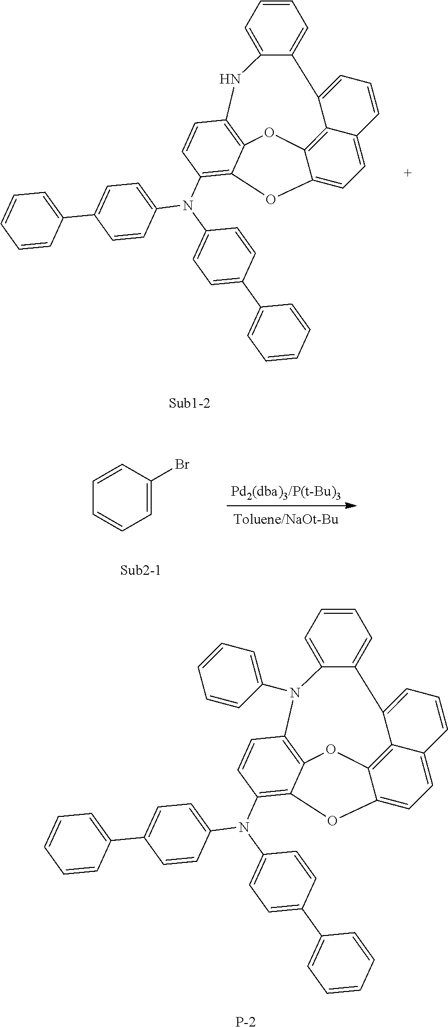

The obtained Sub 1-2 (5.0 g, 7.8 mmol) was placed in a round-bottom flask and dissolved with Toluene (39 mL), and Sub 2-1 (1.2 g, 7.8 mmol), Pd₂(dba)₃ (0.21 g, 0.23 mmol), P(t-Bu)₃ (0.09 g, 0.47 mmol), NaOt-Bu (1.5 g, 15.6 mmol) were added, and an experiment was performed in the same manner as in P-1 to obtain 4.1 g of a product. (Yield 74%)

### 3. Synthesis Example of P-11

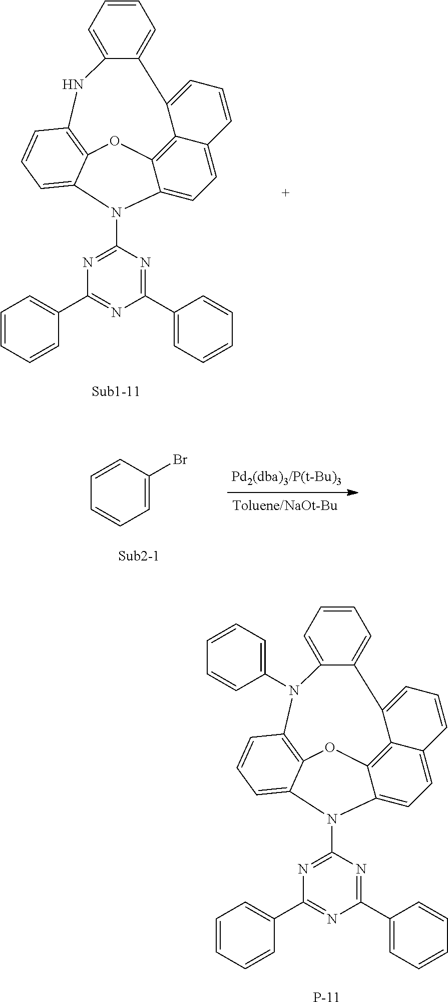

The obtained Sub 1-11 (5.0 g, 9.0 mmol) was placed in a round-bottom flask and dissolved with Toluene (45 mL), Sub 2-1 (1.4 g, 9.0 mmol), Pd₂(dba)₃ (0.25 g, 0.27 mmol), P(t-Bu)₃ (0.11 g, 0.54 mmol), NaOt-Bu (1.7 g, 18.1 mmol) were added, and an experiment was performed in the same manner as in P-1 to obtain 4.0 g of a product. (Yield 71%)

### 4. Synthesis Example of P-21

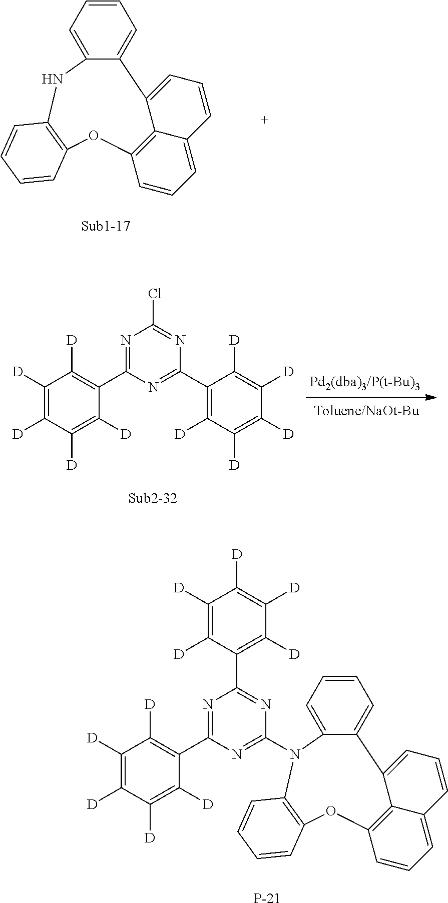

The obtained Sub 1-17 (5.0 g, 16.2 mmol) was placed in a round-bottom flask and dissolved with Toluene (80 mL), and Sub 2-32 (4.5 g, 16.2 mmol), Pd₂(dba)₃ (0.44 g, 0.48 mmol), P(t-Bu)₃ (0.20 g, 0.97 mmol), NaOt-Bu (3.1 g, 32.3 mmol) were added, and an experiment was performed in the same manner as in P-1 to obtain 6.2 g of a product. (Yield 70%)

### 5. Synthesis Example of P-80

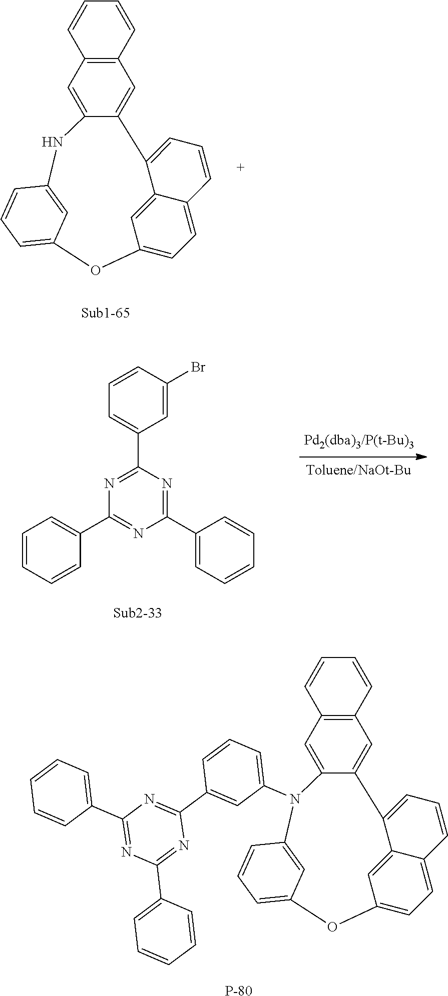

The obtained Sub 1-65 (5.0 g, 13.9 mmol) was placed in a round-bottom flask and dissolved with Toluene (70 mL), and Sub 2-33 (5.4 g, 13.9 mmol), Pd₂(dba)₃ (0.38 g, 0.42 mmol), P(t-Bu)₃ (0.17 g, 0.83 mmol), NaOt-Bu (2.7 g, 27.8 mmol) were added, and an experiment was performed in the same manner as in P-1 to obtain 7.1 g of a product. (Yield 77%)

### 6. Synthesis Example of P-96

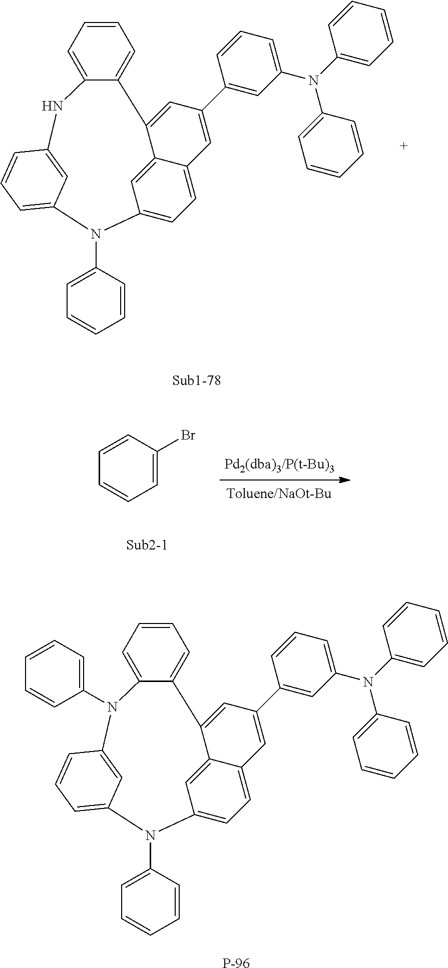

The obtained Sub 1-78 (5.0 g, 8.0 mmol) was placed in a round-bottom flask and dissolved with Toluene (40 mL), and Sub 2-1 (1.3 g, 8.0 mmol), Pd₂(dba)₃ (0.22 g, 0.24 mmol), P(t-Bu)₃ (0.10 g, 0.48 mmol), NaOt-Bu (1.5 g, 15.9 mmol) were added, and an experiment was performed in the same manner as in P-1 to obtain 4.0 g of a product. (Yield 71%)

Meanwhile, the FD-MS values of the compounds P-1 to P-110 of the present invention prepared according to the above synthesis examples are shown in Table 3 below.

Table 3

| compound | FD-MS | compound | FD-MS |
|---|---|---|---|
| P-1 | m/z = 475.16(C₃₄H₂₁NO₂ = 475.55) | P-2 | m/z = 718.26(C₅₂H₃₄N₂O₂ = 718.86) |
| P-3 | m/z = 680.22(C₄₇H₂₈N₄O₂ = 680.77) | P-4 | m/z = 782.26(C₅₆H₃₄N₂O₃ = 782.9) |
| P-5 | m/z = 491.13(C₃₄H₂₁NOS = 491.61) | P-6 | m/z = 698.24(C₄₉H₃₄N₂OS = 698.88) |
| P-7 | m/z = 543.14(C₃₆H₂₁N₃OS = 543.64) | P-8 | m/z = 722.21(C₄₉H₃₀N₄OS = 722.87) |
| P-9 | m/z = 715.26(C₅₂H₃₃N₃O = 715.86) | P-10 | m/z = 837.24(C₅₈H₃₅N₃O₂S = 838) |
| P-11 | m/z = 629.22(C₄₃H₂₇N₅O = 629.72) | P-12 | m/z = 492.16(C₃₄H₂₁FN₂O = 492.55) |
| P-13 | m/z = 595.23(C₄₃H₃₀FNO = 595.72) | P-14 | m/z = 748.25(C₅₃H₃₆N₂OS = 748.94) |
| P-15 | m/z = 675.23(C₄₉H₂₉N₃O = 675.79) | P-16 | m/z = 868.35(C₆₅H₄₄N₂O = 869.08) |
| P-17 | m/z = 461.18(C₃₄H₂₃NO = 461.56) | P-18 | m/z = 658.23(C₄₆H₃₀N₂O₃ = 658.76) |
| P-19 | m/z = 616.23(C₄₃H₂₈N₄O = 616.72) | P-20 | m/z = 567.22(C₄₁H₂₉NO₂ = 567.69) |
| P-21 | m/z = 550.26(C₃₇H₁₄D₁₀N₄O = 550.69) | P-22 | m/z = 537.21 (C₄₀H₂₇NO = 537.66) |
| P-23 | m/z = 678.24(C₄₈H₃₀N₄O = 678.8) | P-24 | m/z = 678.27(C₅₀H₃₄N₂O = 678.84) |
| P-25 | m/z = 565.2(C₄₁H₂₇NO₂ = 565.67) | P-26 | m/z = 669.19(C₄₆H₂₇N₃OS = 669.8) |
| P-27 | m/z = 765.28(C₅₆H₃₅N₃O = 765.92) | P-28 | m/z = 883.36(C₆₅H₄₅N₃O = 884.1) |
| P-29 | m/z = 756.25(C₅₃H₃₂N₄O₂ = 756.87) | P-30 | m/z = 603.26(C₄₅H₃₃NO = 603.77) |
| P-31 | m/z = 761.25(C₅₃H₃₅N₃OS = 761.94) | P-32 | m/z = 692.22(C₄₈H₂₈N₄O₂ = 692.78) |
| P-33 | m/z = 840.23(C₅₈H₃₆N₂OS₂ = 841.06) | P-34 | m/z = 897.3(C₆₄H₃₉N₃O₃ = 898.03) |
| P-35 | m/z = 567.22(C₄₁H₂₉NO₂ = 567.69) | P-36 | m/z = 567.17(C₄₀H₂₅NOS = 567.71) |
| P-37 | m/z = 764.2(C₅₂H₃₂N₂OS₂ = 764.96) | P-38 | m/z = 736.19(C₄₉H₂₈N₄O₂S = 736.85) |
| P-39 | m/z = 764.2(C₅₂H₃₂N₂OS₂ = 764.96) | P-40 | m/z = 532.11(C₃₅H₂₀N₂S₂ = 532.68) |
| P-41 | m/z = 724.2(C₅₀H₃₂N₂S₂ = 724.94) | P-42 | m/z = 730.21(C₄₉H₃₄N₂OS₂ = 730.94) |
| P-43 | m/z = 559.12(C₃₆H₂₁N₃S₂= 559.71) | P-44 | m/z = 839.24(C₅₈H₃₇N₃S₂ = 840.08) |
| P-45 | m/z = 655.26(C₄₃H₁₇D₁₀N₅S = 655.85) | P-46 | m/z = 566.18(C₄₀H₂₆N₂S = 566.72) |
| P-47 | m/z = 667.21(C₄₇H₂₉N₃S = 667.83) | P-48 | m/z = 618.29(C₄₃H₂₂D₁₀N₂S = 618.86) |
| P-49 | m/z = 796.27(C₅₆H₃₆N₄S = 796.99) | P-50 | m/z = 764.23(C₅₃H₃₆N₂S₂ = 765.01) |
| P-51 | m/z = 717.22(C₅₁H₃₁N₃S = 717.89) | P-52 | m/z = 569.22(C₄₁H₃₁NS = 569.77) |
| P-53 | m/z = 820.29(C₆₀H₄₀N₂S = 821.05) | P-54 | m/z = 605.19(C₄₂H₂₇N₃S = 605.76) |
| P-55 | m/z = 541.15(C₃₈H₂₃NOS = 541.67) | P-56 | m/z = 796.29(C₅₈H₄₀N₂S = 797.03) |
| P-57 | m/z = 659.17(C₄₆H₂₉NS₂ = 659.87) | P-58 | m/z = 599.17(C₄₁H₂₉NS₂ = 599.81) |
| P-59 | m/z = 632.2(C₄₃H₂₈N₄S = 632.79) | P-60 | m/z = 738.18(C₅₀H₃₀N₂OS₂ = 738.92) |
| P-61 | m/z = 685.16(C₄₆H₂₇N₃S₂ = 685.86) | P-62 | m/z = 745.23(C₅₁H₃₁N₅S = 745.9) |
| P-63 | m/z = 934.34(C₆₉H₄₆N₂S = 935.2) | P-64 | m/z = 694.24(C₅₀H₃₄N₂S = 694.9) |
| P-65 | m/z = 800.29(C₅₇H₄₀N₂OS = 801.02) | P-66 | m/z = 683.17(C₄₆H₂₅N₃O₂S = 683.79) |
| P-67 | m/z = 869.22(C₅₈H₃₅N₃O₂S₂ = 870.06) | P-68 | m/z = 790.22(C₅₃H₃₄N₄S₂ = 791) |
| P-69 | m/z = 801.23(C₅₅H₃₅N₃S₂ = 802.03) | P-70 | m/z = 733.26(C₅₂H₃₅N₃S = 733.93) |
| P-71 | m/z = 491.13(C₃₄H₂₁NOS = 491.61) | P-72 | m/z = 704.28(C₅₂H₃₆N₂O = 704.87) |
| P-73 | m/z = 652.25(C₄₈H₃₂N₂O = 652.8) | P-74 | m/z = 750.27(C₅₃H₃₈N₂OS = 750.96) |
| P-75 | m/z = 692.25(C₅₀H₃₂N₂O₂ = 692.82) | P-76 | m/z = 716.29(C₅₀H₄₀N₂OS = 716.94) |
| P-77 | m/z = 540.2(C₃₇H₂₄N₄O = 540.63) | P-78 | m/z = 589.22(C₄₂H₂₇N₃O = 589.7) |
| P-79 | m/z = 589.22(C₄₂H₂₇N₃O = 589.7) | P-80 | m/z = 666.24(C₄₇H₃₀N₄O = 666.78) |
| P-81 | m/z = 553.19(C₄₀H₂₇NS = 553.72) | P-82 | m/z = 658.21(C₄₆H₃₀N₂OS = 658.82) |
| P-83 | m/z = 796.29(C₅₈H₄₀N₂S = 797.03) | P-84 | m/z = 644.23(C₄₆H₃₂N₂S = 644.84) |
| P-85 | m/z = 619.23(C₄₅H₃₃NS = 619.83) | P-86 | m/z = 658.21(C₄₆H₃₀N₂OS = 658.82) |
| P-87 | m/z = 556.17(C₃₇H₂₄N₄S = 556.69) | P-88 | m/z = 605.19(C₄₂H₂₇N₃S = 605.76) |
| P-89 | m/z = 605.19(C₄₂H₂₇N₃S = 605.76) | P-90 | m/z = 721.23(C₄₉H₃₁N₅S = 721.88) |
| P-91 | m/z = 612.26(C₄₆H₃₂N₂ = 612.78) | P-92 | m/z = 612.26(C₄₆H₃₂N₂ = 612.78) |
| P-93 | m/z = 783.27(C₅₆H₃₇N₃S = 783.99) | P-94 | m/z = 839.24(C₅₈H₃₇N₃S₂ = 840.08) |
| P-95 | m/z = 897.32(C₆₅H₄₃N₃S = 898.14) | P-96 | m/z = 703.3(C₅₂H₃₇N₃ = 703.89) |
| P-97 | m/z = 651.22(C₄₃H₂₇F₂N₅ = 651.72) | P-98 | m/z = 669.29(C₄₈H₂₇D₅N₄ = 669.84) |
| P-99 | m/z = 715.27(C₅₁H₃₃N₅ = 715.86) | P-100 | m/z = 610.24(C₄₆H₃₀N₂ = 610.76) |
| P-101 | m/z = 711.29(C₅₅H₃₇N = 711.91) | P-102 | m/z = 770.37(C₅₈H₄₆N₂ = 771.02) |
| P-103 | m/z = 810.31(C₅₉H₄₂N₂S = 811.06) | P-104 | m/z = 704.32(C₅₃H₄₀N₂ = 704.92) |
| P-105 | m/z = 852.35(C₆₅H₄₄N₂ = 853.08) | P-106 | m/z = 566.25(C₄₀H₃₀N₄ = 566.71) |
| P-107 | m/z = 619.32(C₄₇H₄₁N = 619.85) | P-108 | m/z = 665.28(C₄₉H₃₅N₃ = 665.84) |
| P-109 | m/z = 716.32(C₅₄H₄₀N₂ = 716.93) | P-110 | m/z = 767.33(C₅₇H₄₁N₃ = 67.98) |

Manufacturing Evaluation of Organic Electronic Element

### [Example 1] Red Organic Light Emitting Device (Emitting-Auxiliary Layer)

An organic electroluminescent device was manufactured according to a conventional method using the compound of the present invention as a light emitting-auxiliary layer material. First, on the ITO layer (anode) formed on the glass substrate, N1-(naphthalen-2-yl)-N4,N4-bis(4-(naphthalen-2-yl(phenyl)amino)phenyl)-N1-phenylbenzene-1,4-diamine (abbreviated as 2-TNATA) film as a hole injection layer was vacuum-deposited to form a thickness of 60 nm. Then, 4,4-bis[N-(1-naphthyl)-N-phenylamino]biphenyl (abbreviated as NPD) as a hole transport compound was vacuum-deposited on the film to a thickness of 60 nm to form a hole transport layer. Then, as a material for the emitting-auxiliary layer, the compound P-1 of the present invention was vacuum-deposited to a thickness of 20 nm to form an emitting-auxiliary layer.

After forming the emitting-auxiliary layer, an emitting layer with a thickness of 30 nm was deposited on the emitting-auxiliary layer by doping CBP[4,4′-N,N′-dicarbazole-biphenyl] as a host and (piq)2Ir(acac)[bis-(1-phenyl isoquinolyl)iridium(III)acetylacetonate] as a dopant at a weight of 95:5. Vacuum-depositing (1,1′-bisphenyl)-4-oleato)bis(2-methyl-8-quinolineoleato)aluminum (abbreviated as BAlq) as a hole blocking layer to a thickness of 10 nm, and as an electron transport layer, tris(8-quinolinol)aluminum (abbreviated as Alq3) was deposited to a thickness of 40 nm. Then, as an electron injection layer, LiF, an alkali metal halide was deposited to a thickness of 0.2 nm, then, Al was deposited to a thickness of 150 nm and used as a cathode to prepare an organic electroluminescent device.

### [Example 2] to [Example 10] Red Organic Light Emitting Device

An organic electroluminescent device was manufactured in the same manner as in Example 1, except that the compound of the present invention described in Table 4 was used instead of the compound P-1 of the present invention as a light emitting-auxiliary layer material.

### Comparative Example 1

An organic electroluminescent device was manufactured in the same manner as in Example 1, except that the emitting-auxiliary layer was not used.

### Comparative Example 2

An organic electroluminescent device was manufactured in the same manner as in Example 1, except that the following comparative compound A was used instead of the compound P-1 of the present invention as a light emitting-auxiliary layer material.

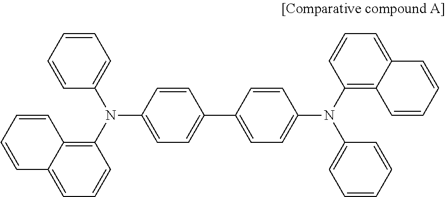 [SMILES: c1ccc(N(c2ccc(-c3ccc(N(c4ccccc4)c4cccc5ccccc45)cc3)cc2)c2cccc3ccccc23)cc1]

By applying a forward bias DC voltage to the organic electroluminescent devices prepared in Examples and Comparative Examples prepared in this way, Electroluminescence (EL) characteristics were measured with PR-650 from photo research, and as a result of the measurement, the T95 lifetime was measured using a lifetime measuring device manufactured by McScience at 2500 cd/m² standard luminance. Table 4 below shows the device fabrication and evaluation results.

TABLE 4

| | | | Current | | | | | |
|---|---|---|---|---|---|---|---|---|
| | | | Density | Brightness | Efficiency | | CIE | |
| | compound | Voltage | (mA/cm²) | (cd/m²) | (cd/A) | T(95) | x | y |
| comparative | — | 6.7 | 34.2 | 2500 | 7.3 | 62.9 | 0.60 | 0.30 |
| example(1) | | | | | | | | |
| comparative | comparative | 6.3 | 20.0 | 2500 | 12.5 | 87.6 | 0.62 | 0.32 |
| example(2) | compound A | | | | | | | |
| example(1) | compound | 5.6 | 11.2 | 2500 | 22.4 | 104.5 | 0.65 | 0.33 |
| | (P-1) | | | | | | | |
| example(2) | Compound | 5.4 | 10.7 | 2500 | 23.4 | 109.7 | 0.62 | 0.34 |
| | (P-2) | | | | | | | |
| example(3) | Compound | 5.3 | 10.3 | 2500 | 24.2 | 111.9 | 0.62 | 0.33 |
| | (P-4) | | | | | | | |
| example(4) | Compound | 5.3 | 10.8 | 2500 | 23.1 | 107.6 | 0.62 | 0.30 |
| | (P-6) | | | | | | | |
| example(5) | Compound | 5.3 | 10.6 | 2500 | 23.5 | 105.6 | 0.61 | 0.34 |
| | (P-24) | | | | | | | |
| example(6) | Compound | 5.2 | 10.4 | 2500 | 24.1 | 108.7 | 0.65 | 0.32 |
| | (P-37) | | | | | | | |
| example(7) | Compound | 5.2 | 10.7 | 2500 | 23.3 | 104.6 | 0.60 | 0.31 |
| | (P-41) | | | | | | | |
| example(8) | Compound | 5.0 | 10.9 | 2500 | 23.0 | 105.7 | 0.60 | 0.34 |
| | (P-42) | | | | | | | |
| example(9) | Compound | 5.3 | 11.3 | 2500 | 22.2 | 101.6 | 0.61 | 0.34 |
| | (P-86) | | | | | | | |
| example(10) | Compound | 5.7 | 11.7 | 2500 | 21.3 | 98.6 | 0.64 | 0.34 |
| | (P-96) | | | | | | | |

Referring to Table 4, when a red organic light emitting device is manufactured using the material for an organic electroluminescent device of the present invention as a light emitting-auxiliary layer material, it is possible to significantly improve the luminous efficiency and lifespan of the organic electroluminescent device compared to the comparative example in which the emitting-auxiliary layer is not used or Comparative Compound A is used.

Consequently, it can be seen that the results of Comparative Example 2 using Comparative Compound A were better than Comparative Example 1 without using the emitting-auxiliary layer, and the results of Examples 1 to 10 of the compound of the present invention are remarkably excellent compared to that of Comparative Compound A.

When the compound of the present invention is compared with the comparative compound A, the structures of the two compounds are significantly different, and the physical properties (such as the energy level (especially the HOMO level)) of the two compounds are different. Therefore, as the physical properties of the compound change, the compound of the present invention improves the charge balance in the emitting layer, so that light emission is better achieved inside the emitting layer rather than the interface of the emitting layer. As a result, it is judged that deterioration at the interface of the emitting layer is also reduced, and driving voltage, efficiency, and lifespan seem to be improved. Therefore, it is judged that this point acts as a major factor in improving device performance during device deposition.

### [Example 11] Red Organic Light Emitting Device (Phosphorescent Host)

An organic electroluminescent device was manufactured according to a conventional method using the compound of the present invention as a phosphorescent host material. First, on an ITO layer (anode) formed on a glass substrate, N1-(naphthalen-2-yl)-N4,N4-bis(4-(naphthalen-2-yl(phenyl)amino)phenyl)-N1-phenylbenzene-1,4-diamine (abbreviated as 2-TNATA) film as a hole injection layer was vacuum-deposited to form a thickness of 60 nm. Then, 4,4-bis[N-(1-naphthyl)-N-phenylamino]biphenyl(abbreviated as NPB) as a hole transport compound was vacuum-deposited on the film to a thickness of 60 nm to form a hole transport layer. After forming the hole transport layer, an emitting layer with a thickness of 30 nm was deposited on the hole transport layer by doping the compound P-8 of the present invention, as a host and (piq)2Ir(acac)[bis-(1-phenyl isoquinolyl)iridium(III)acetylacetonate] as a dopant at a weight of 95:5. Vacuum-depositing (1,1′-bisphenyl)-4-oleato)bis(2-methyl-8-quinolineoleato)aluminum (abbreviated as BAlq) as a hole blocking layer to a thickness of 10 nm, and as an electron transport layer, tris(8-quinolinol)aluminum (abbreviated as Alq3) was deposited to a thickness of 40 nm. Then, as an electron injection layer, LiF, an alkali metal halide, was deposited to a thickness of 0.2 nm, then, Al was deposited to a thickness of 150 nm and used as a cathode to prepare an organic electroluminescent device.

### [Example 12] to [Example 22] Red Organic Light Emitting Device (Host)

An organic electroluminescent device was manufactured in the same manner as in Example 11, except that the compound of the present invention shown in Table 5 was used instead of the compound P-8 of the present invention as a host material.

### Comparative Example 3

An organic electroluminescent device was manufactured in the same manner as in Example 11, except that the following comparative compound B was used instead of the compound P-8 of the present invention as a host material.

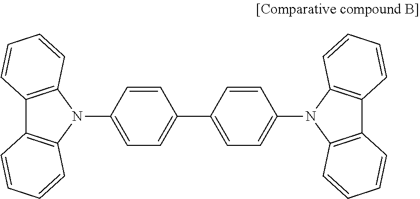 [SMILES: c1ccc2c(c1)c1ccccc1n2-c1ccc(-c2ccc(-n3c4ccccc4c4ccccc43)cc2)cc1]

By applying a forward bias DC voltage to the organic electroluminescent devices prepared in Examples and Comparative Examples prepared in this way, Electroluminescence (EL) characteristics were measured with PR-650 from photo research, and as a result of the measurement, the T95 lifetime was measured using a lifetime measuring device manufactured by McScience at 2500 cd/m² standard luminance. Table 5 below shows the device fabrication and evaluation results.

TABLE 5

| | | | Current | | | | | |
|---|---|---|---|---|---|---|---|---|
| | | | Density | Brightness | Efficiency | | CIE | |
| | compound | Voltage | (mA/cm²) | (cd/m²) | (cd/A) | T(95) | x | y |
| Comparative | Comparative | 6.7 | 34.2 | 2500 | 7.3 | 75.6 | 0.65 | 0.33 |
| example(3) | compound B | | | | | | | |
| Example(11) | Compound | 5.3 | 9.4 | 2500 | 26.7 | 118.7 | 0.64 | 0.31 |
| | (P-8) | | | | | | | |
| Example(12) | Compound | 5.3 | 10.1 | 2500 | 24.7 | 110.9 | 0.62 | 0.35 |
| | (P-11) | | | | | | | |
| Example(13) | Compound | 5.1 | 9.6 | 2500 | 26.2 | 112.0 | 0.64 | 0.32 |
| | (P-21) | | | | | | | |
| Example(14) | Compound | 5.1 | 9.1 | 2500 | 27.5 | 117.6 | 0.62 | 0.33 |
| | (P-38) | | | | | | | |
| Example(15) | Compound | 5.1 | 9.8 | 2500 | 25.4 | 112.1 | 0.60 | 0.33 |
| | (P-43) | | | | | | | |
| Example(16) | Compound | 5.4 | 9.6 | 2500 | 25.9 | 108.8 | 0.62 | 0.32 |
| | (P-49) | | | | | | | |
| Example(17) | Compound | 5.1 | 9.9 | 2500 | 25.2 | 105.6 | 0.64 | 0.31 |
| | (P-62) | | | | | | | |
| Example(18) | Compound | 5.5 | 9.7 | 2500 | 25.8 | 109.7 | 0.65 | 0.33 |
| | (P-79) | | | | | | | |
| Example(19) | Compound | 5.3 | 9.9 | 2500 | 25.3 | 107.6 | 0.62 | 0.35 |
| | (P-80) | | | | | | | |
| Example(20) | Compound | 5.2 | 10.2 | 2500 | 24.6 | 107.7 | 0.64 | 0.31 |
| | (P-87) | | | | | | | |
| Example(21) | Compound | 5.4 | 10.5 | 2500 | 23.7 | 104.6 | 0.64 | 0.30 |
| | (P-99) | | | | | | | |
| Example(22) | Compound | 5.5 | 10.3 | 2500 | 24.2 | 103.6 | 0.60 | 0.33 |
| | (P-106) | | | | | | | |

Referring to Table 5, when a red organic electroluminescent device is manufactured using the material for an organic electroluminescent device of the present invention as a host material, the driving voltage, efficiency and lifespan of the organic electroluminescent device can be improved compared to Comparative Example 3 using Comparative Compound B.

When the compound of the present invention is compared with the comparative compound B, the structures of the two compounds are significantly different, and the physical properties (such as energy level (especially LUMO level)) of the two compounds are different. Therefore, as the physical properties of the compound change, the compound of the present invention improves the charge balance in the emitting layer, so that light emission is better achieved inside the emitting layer rather than the interface of the emitting layer. As a result, deterioration at the interface of the emitting layer is also reduced, and the driving voltage, efficiency, and lifespan are judged to be improved. Therefore, it is judged that this point acts as a major factor in improving device performance during device deposition.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

### BRIEF DESCRIPTION OF THE DRAWINGS

| | |
|---|---|
| 100, 200, 300: organic electronic | 110: the first electrode |
| element | 130: hole transport layer |
| 120: hole injection layer | 150: electron transport layer |
| 140: emitting layer | 170: second electrode |
| 160: electron injection layer | 210: buffer layer |
| 180: light efficiency enhancing Layer | 320: first hole injection layer |
| 220: emitting-auxiliary layer | 340: first emitting layer |
| 330: first hole transport layer | 360: first charge generation layer |
| 350: first electron transport layer | 420: second hole injection layer |
| 361: second charge generation layer | 440: second emitting layer |
| 430: second hole transport layer | CGL: charge generation layer |
| 450: second electron transport layer | ST2: second stack |
| ST1: first stack | |

What is claimed is:

1. A compound represented by Formula 1: 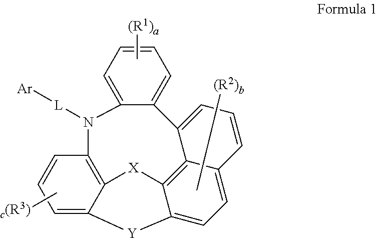
wherein,
1) X and Y are independently of each other are O, S, NR′, CR′R″ or absent;
2) R′ and R″ are independently of each other hydrogen; deuterium; halogen; C₁-C₆₀ alkyl group; a C₂-C₆₀ alkenyl group; a C₂-C₆₀ alkynyl group; a C₁-C₆₀ alkoxy group; a C₆-C₆₀ aryloxy group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; or R and R″ may be bonded to each other to form a spiro;
3) R¹, R² and R³ are each the same or different, and each independently selected from the group consisting of hydrogen; deuterium; halogen; cyano group; nitro group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₆₀ alkyl group; a C₂-C₆₀ alkenyl group; a C₂-C₆₀ alkynyl group; a C₁-C₆₀ alkoxy group; a C₆-C₆₀ aryloxy group; and -L′-N(R^{a})(R^{b}); or a plurality of adjacent R¹s, or a plurality of R²s, or a plurality of R³s may be bonded to each other to form a ring;
4) L and L′ are each independently selected from the group consisting of single bond; a C₆-C₆₀ arylene group; a fluorenylene group; a C₂-C₆₀ heterocyclic group including at least one heteroatom of O, N, S, Si or P; and a C₃-C₆₀ aliphatic ring;
5) Ar, R^{a} and R^{b} are each independently a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring;
6) a is an integer of 0 to 4, b is an integer of 0 to 5, and c is an integer of 0 to 3;
7) the aryl group, arylene group, heterocyclic group, fluorenyl group, fluorenylene group, fused ring group, alkyl group, alkenyl group, alkoxy group or aryloxy group of R′, R″, R¹, R², R³, L, L′, Ar, R^{a}, or R^{b} may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxy group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group; C₈-C₂₀ arylalkenyl group; and -L′-NR^{a}R^{b}; and the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

2. The compound of claim 1, wherein the compound represented by Formula 1 is represented by one of Formulas 2 to 4: 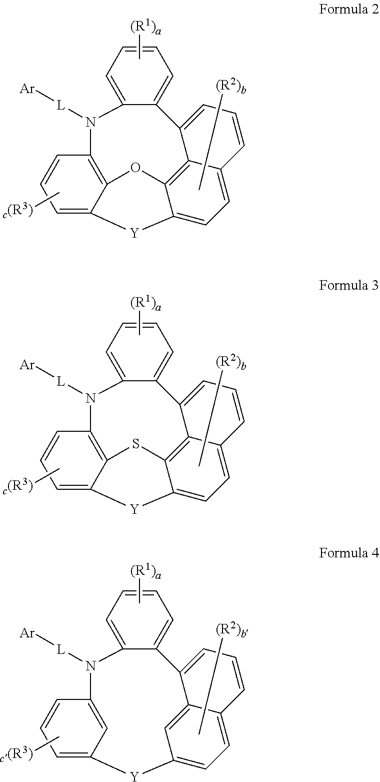
wherein:
1) Y, L, Ar, R¹, R², R³, a, b and c are the same as defined in Formula 1, and
2) b′ is an integer from 0 to 6, and c′ is an integer from 0 to 4.

3. The compound of claim 1, wherein the compound represented by Formula 1 is represented by one of Formulas 5 to 9: 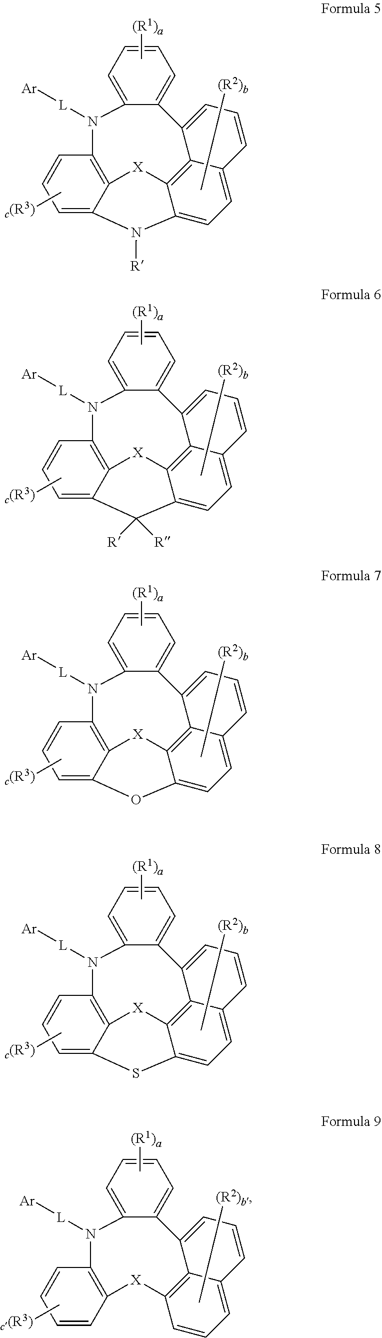
wherein:
1) X, L, Ar, R¹, R², R³, R′, R″, a, b and c are the same as defined in Formula 1; and
2) b′ is an integer from 0 to 6, and c′ is an integer from 0 to 4.

4. The compound of claim 1, wherein Ar of Formula 1 is represented by Formula (A): 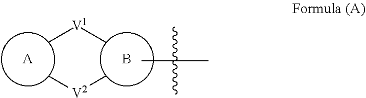
wherein
1) A and B are each independently a C₆-C₂₀ aryl group; or C₄-C₂₀ heterocyclic group;
2) V¹ and V² are each independently a single bond, O, S, NR′ or CR′R″, with the proviso that both V¹ and V² are not single bonds at the same time;
3) R and R″ are as defined in claim 1; and
4) [symbol] indicates the binding position.

5. The compound of claim 1, wherein at least one of R¹ to R³ in Formula 1 is represented by Formula (B) or Formula (C): 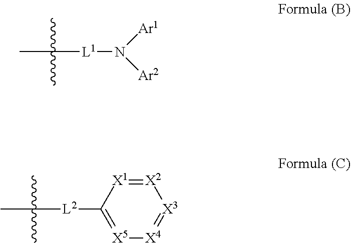
wherein
1) L¹ and L² are the same as the definition of L in claim 1,
2) Ar¹ and Ar² are the same as the definition of Ar in claim 1,
3) X¹, X², X³, X⁴ and X⁵ are independently of each other CRx or N, with the proviso that at least one of X¹, X², X³, X⁴ and X⁵ is N,
4) Rx is selected from the group consisting of hydrogen; deuterium; halogen; cyano group; nitro group; a C₆-C₆₀ aryl group; or a C₂-C₆₀ heterocyclic group including at least one hetero atom of O, N, S, Si or P; a fluorenyl group; a fused ring group of a C₃-C₆₀ aliphatic ring and a C₆-C₆₀ aromatic ring; C₁-C₆₀ alkyl group; a C₂-C₆₀ alkenyl group; a C₂-C₆₀ alkynyl group; a C₁-C₆₀ alkoxyl group; and a C₆-C₆₀ aryloxy group, wherein a plurality of adjacent Rx may be bonded to each other to form a ring, and
5) [symbol] indicates the binding position.

6. The compound of claim 1, wherein the compound represented by Formula 1 is any one of the following compounds:

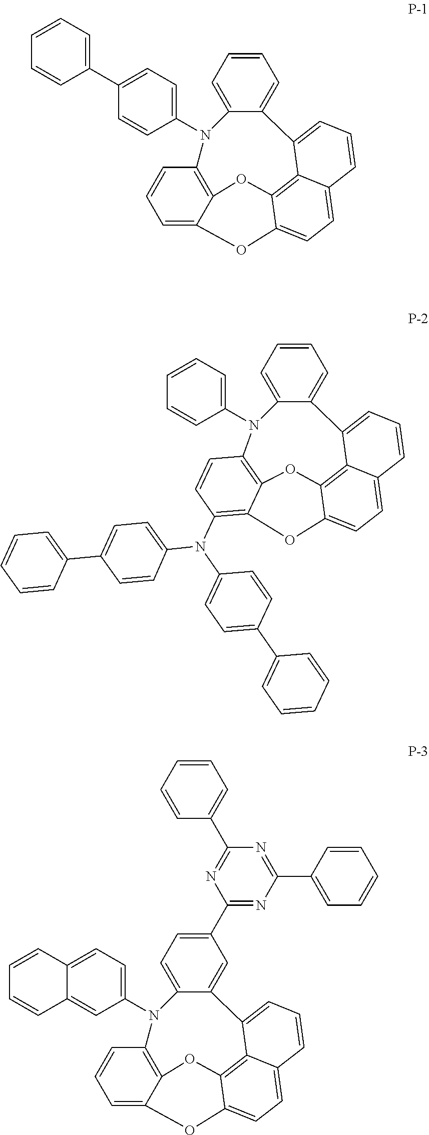 [SMILES: c1ccc(-c2ccc(N3c4ccccc4-c4cccc5ccc6c(c45)Oc4c(cccc43)O6)cc2)cc1 ; c1ccc(-c2ccc(N(c3ccc(-c4ccccc4)cc3)c3ccc4c5c3Oc3ccc6cccc(c6c3O5)-c3ccccc3N4c3ccccc3)cc2)cc1 ; c1ccc(-c2nc(-c3ccccc3)nc(-c3ccc4c(c3)-c3cccc5ccc6c(c35)Oc3c(cccc3N4c3ccc4ccccc4c3)O6)n2)cc1]

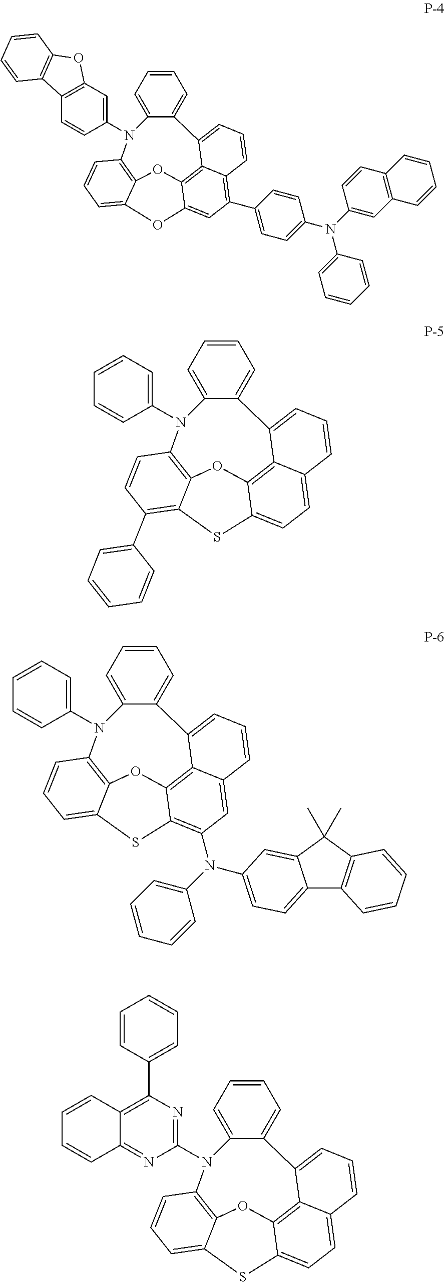

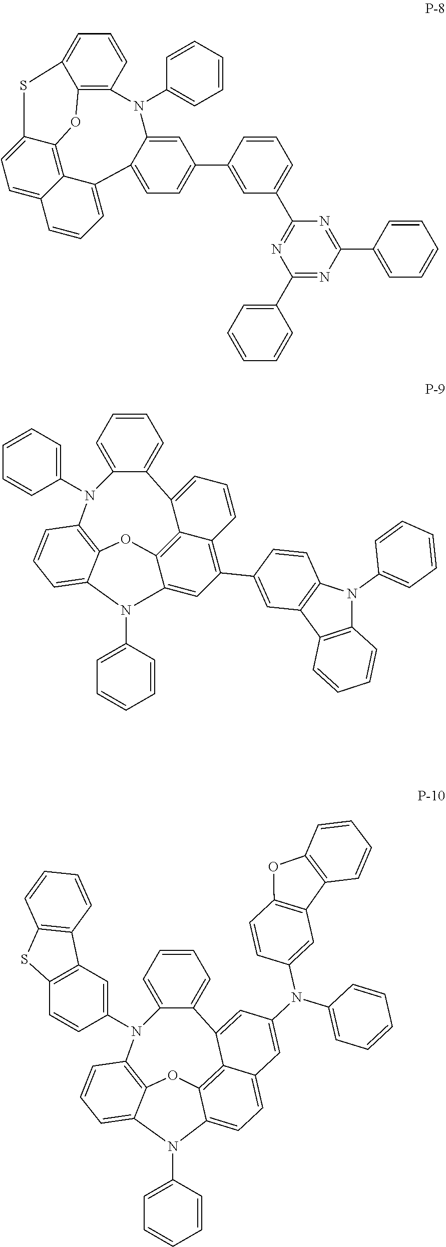 [SMILES: c1ccc(-c2nc(-c3ccccc3)nc(-c3cccc(-c4ccc5c(c4)N(c4ccccc4)c4cccc6c4Oc4c(ccc7cccc-5c47)S6)c3)n2)cc1 ; c1ccc(-n2c3ccccc3c3cc(-c4cc5c6oc7c(cccc7n5-c5ccccc5)n(-c5ccccc5)c5ccccc5c5cccc4c56)ccc32)cc1 ; c1ccc(N(c2ccc3oc4ccccc4c3c2)c2cc3ccc4c5oc6c(cccc6n4-c4ccccc4)n(-c4ccc6sc7ccccc7c6c4)c4ccccc4c(c2)c35)cc1]

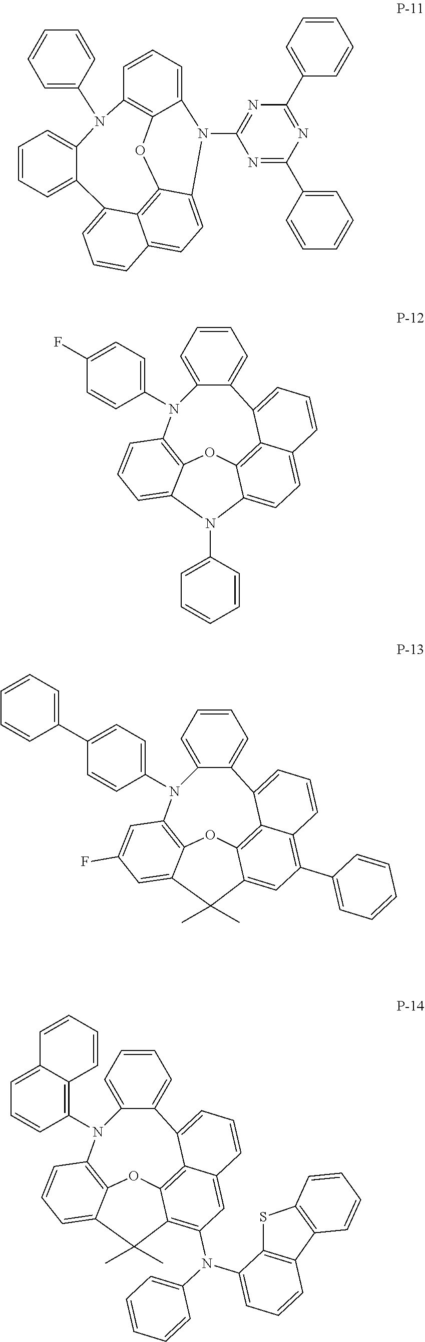 [SMILES: c1ccc(-c2nc(-c3ccccc3)nc(-n3c4ccc5cccc6c7ccccc7n(-c7ccccc7)c7cccc3c7oc4c56)n2)cc1 ; Fc1ccc(-n2c3ccccc3c3cccc4ccc5c(oc6c2cccc6n5-c2ccccc2)c43)cc1 ; CC1(C)c2cc(F)cc3c2Oc2c1cc(-c1ccccc1)c1cccc(c21)-c1ccccc1N3c1ccc(-c2ccccc2)cc1 ; CC1(C)c2cccc3c2Oc2c1c(N(c1ccccc1)c1cccc4c1sc1ccccc14)cc1cccc(c21)-c1ccccc1N3c1cccc2ccccc12]

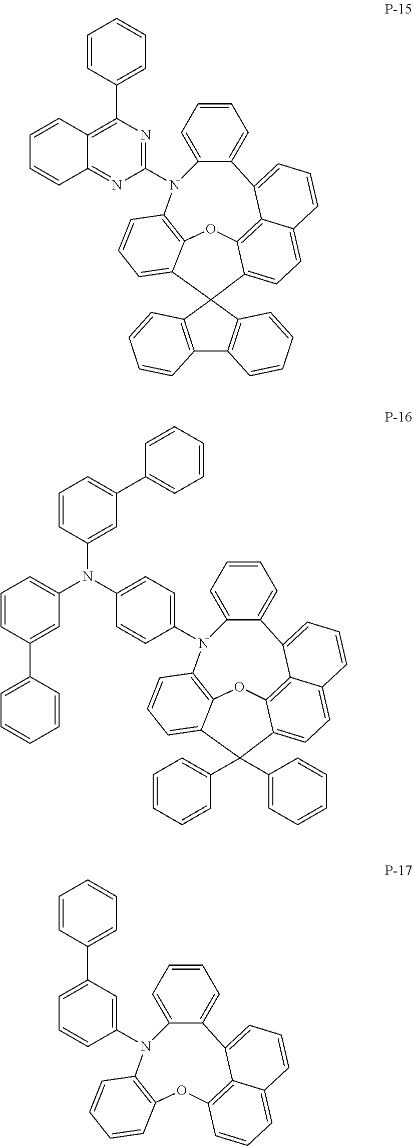 [SMILES: c1ccc(-c2nc(N3c4ccccc4-c4cccc5ccc6c(c45)Oc4c3cccc4C63c4ccccc4-c4ccccc43)nc3ccccc23)cc1 ; c1ccc(-c2cccc(N(c3ccc(N4c5ccccc5-c5cccc6ccc7c(c56)Oc5c4cccc5C7(c4ccccc4)c4ccccc4)cc3)c3cccc(-c4ccccc4)c3)c2)cc1 ; c1ccc(-c2cccc(N3c4ccccc4Oc4cccc5cccc(c45)-c4ccccc43)c2)cc1]

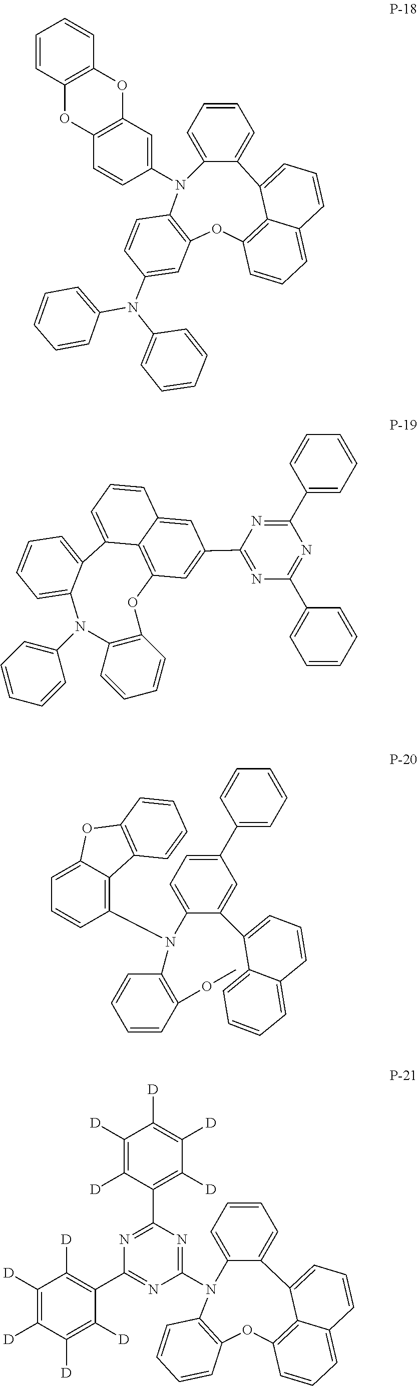 [SMILES: c1ccc(N(c2ccccc2)c2ccc3c(c2)Oc2cccc4cccc(c24)-c2ccccc2N3c2ccc3c(c2)Oc2ccccc2O3)cc1 ; c1ccc(-c2nc(-c3ccccc3)nc(-c3cc4c5c(cccc5c3)-c3ccccc3N(c3ccccc3)c3ccccc3O4)n2)cc1 ; COc1ccccc1N(c1ccc(-c2ccccc2)cc1-c1cccc2ccccc12)c1cccc2oc3ccccc3c12 ; [2H]c1c([2H])c([2H])c(-c2nc(-c3c([2H])c([2H])c([2H])c([2H])c3[2H])nc(N3c4ccccc4Oc4cccc5cccc(c45)-c4ccccc43)n2)c([2H])c1[2H]]

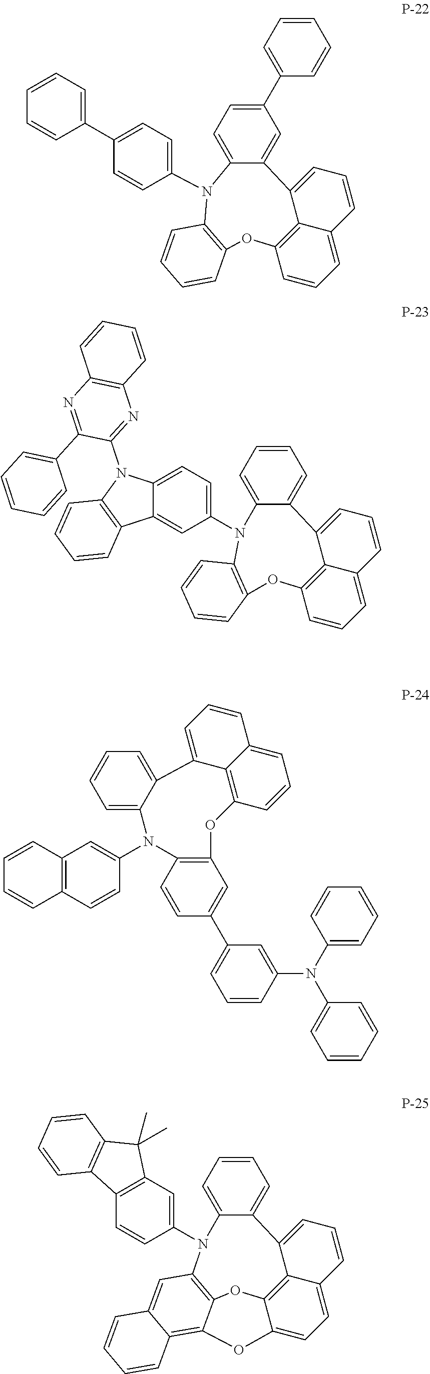 [SMILES: c1ccc(-c2ccc(N3c4ccccc4Oc4cccc5cccc(c45)-c4cc(-c5ccccc5)ccc43)cc2)cc1 ; c1ccc(-c2nc3ccccc3nc2-n2c3ccccc3c3cc(N4c5ccccc5Oc5cccc6cccc(c56)-c5ccccc54)ccc32)cc1 ; c1ccc(N(c2ccccc2)c2cccc(-c3ccc4c(c3)Oc3cccc5cccc(c35)-c3ccccc3N4c3ccc4ccccc4c3)c2)cc1 ; CC1(C)c2ccccc2-c2ccc(N3c4ccccc4-c4cccc5ccc6c(c45)Oc4c3cc3ccccc3c4O6)cc21]

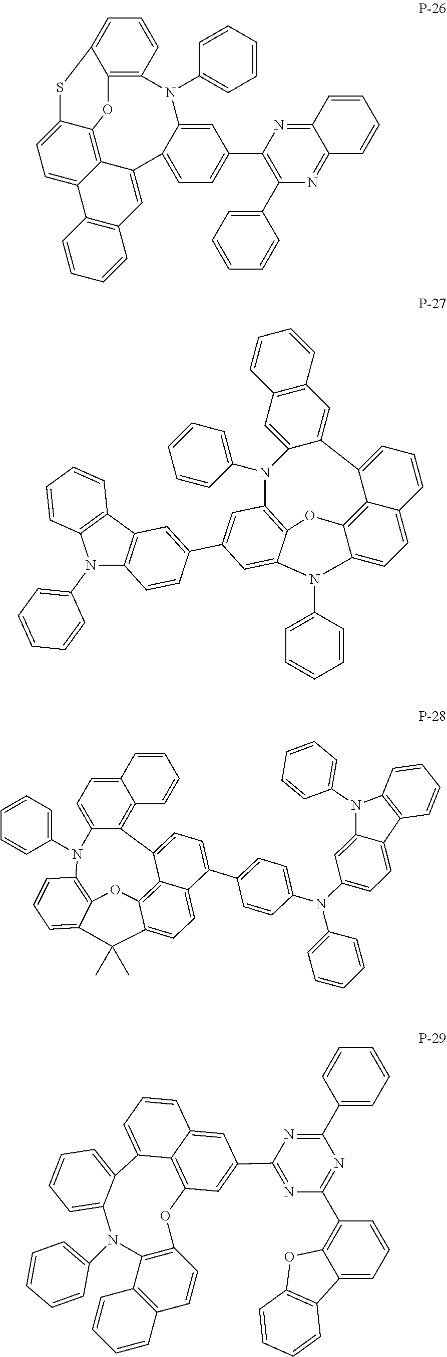 [SMILES: c1ccc(-c2nc3ccccc3nc2-c2ccc3c(c2)N(c2ccccc2)c2cccc4c2Oc2c(ccc5c2c-3cc2ccccc25)S4)cc1 ; c1ccc(-n2c3ccccc3c3cc(-c4cc5c6oc7c8c(cccc8c8cc9ccccc9cc8n5-c5ccccc5)ccc7n(-c5ccccc5)c6c4)ccc32)cc1 ; CC1(C)c2cccc3c2Oc2c1ccc1c(-c4ccc(N(c5ccccc5)c5ccc6c7ccccc7n(-c7ccccc7)c6c5)cc4)ccc(c21)-c1c(ccc2ccccc12)N3c1ccccc1 ; c1ccc(-c2nc(-c3cc4c5c(cccc5c3)-c3ccccc3N(c3ccccc3)c3c(ccc5ccccc35)O4)nc(-c3cccc4c3oc3ccccc34)n2)cc1]

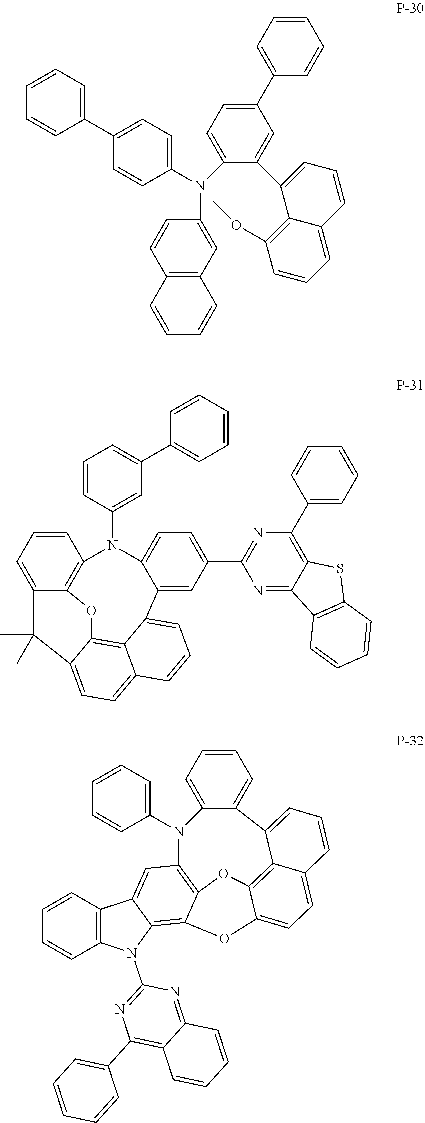 [SMILES: COc1cccc2cccc(-c3cc(-c4ccccc4)ccc3N(c3ccc(-c4ccccc4)cc3)c3ccc4ccccc4c3)c12 ; CC1(C)c2cccc3c2Oc2c1ccc1cccc(c21)-c1cc(-c2nc(-c4ccccc4)c4sc5ccccc5c4n2)ccc1N3c1cccc(-c2ccccc2)c1 ; c1ccc(-c2nc(-n3c4ccccc4c4cc5c6c(c43)Oc3ccc4cccc(c4c3O6)-c3ccccc3N5c3ccccc3)nc3ccccc23)cc1]

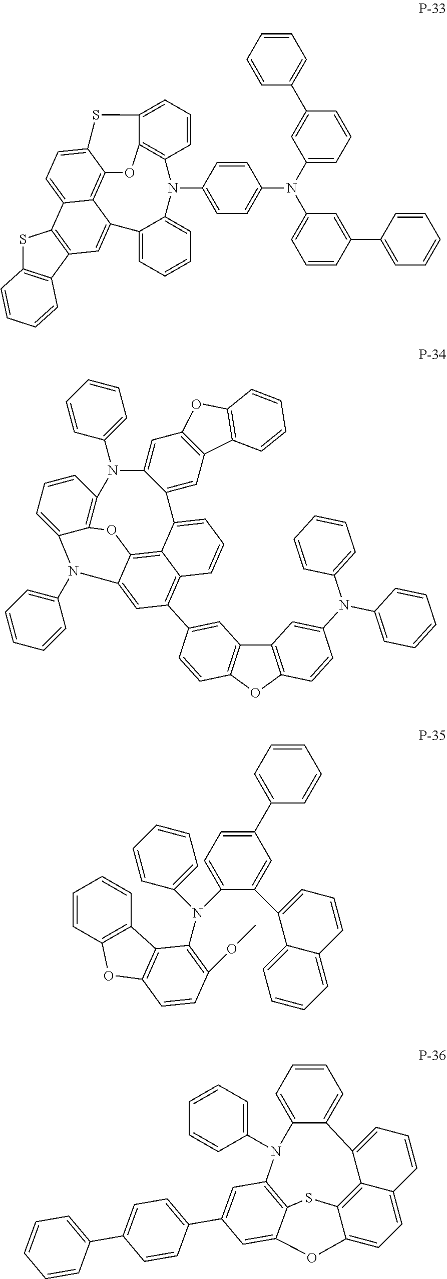 [SMILES: c1ccc(-c2cccc(N(c3ccc(N4c5ccccc5-c5cc6c7ccccc7sc6c6ccc7c(c56)Oc5c(cccc54)S7)cc3)c3cccc(-c4ccccc4)c3)c2)cc1 ; c1ccc(N(c2ccccc2)c2ccc3oc4ccc(-c5cc6c7oc8c(cccc8n6-c6ccccc6)n(-c6ccccc6)c6cc8oc9ccccc9c8cc6c6cccc5c67)cc4c3c2)cc1 ; COc1ccc2oc3ccccc3c2c1N(c1ccccc1)c1ccc(-c2ccccc2)cc1-c1cccc2ccccc12 ; c1ccc(-c2ccc(-c3cc4c5c(c3)N(c3ccccc3)c3ccccc3-c3cccc6ccc(c(c36)S5)O4)cc2)cc1]

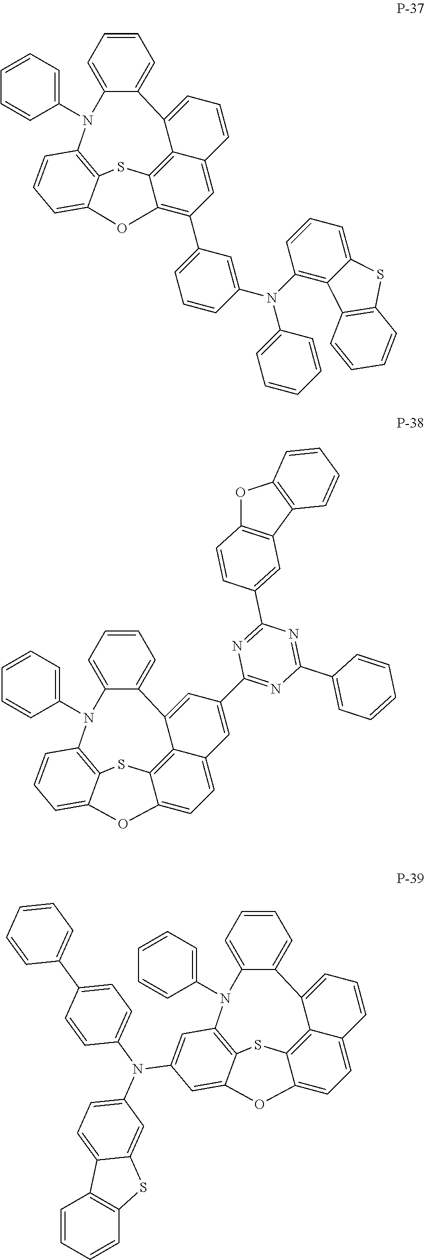 [SMILES: c1ccc(N2c3ccccc3-c3cccc4cc(-c5cccc(N(c6ccccc6)c6cccc7sc8ccccc8c67)c5)c5c(c34)Sc3c(cccc32)O5)cc1 ; c1ccc(-c2nc(-c3cc4c5c6c(ccc5c3)Oc3cccc(c3S6)N(c3ccccc3)c3ccccc3-4)nc(-c3ccc4oc5ccccc5c4c3)n2)cc1 ; c1ccc(-c2ccc(N(c3cc4c5c(c3)N(c3ccccc3)c3ccccc3-c3cccc6ccc(c(c36)S5)O4)c3ccc4c(c3)sc3ccccc34)cc2)cc1]

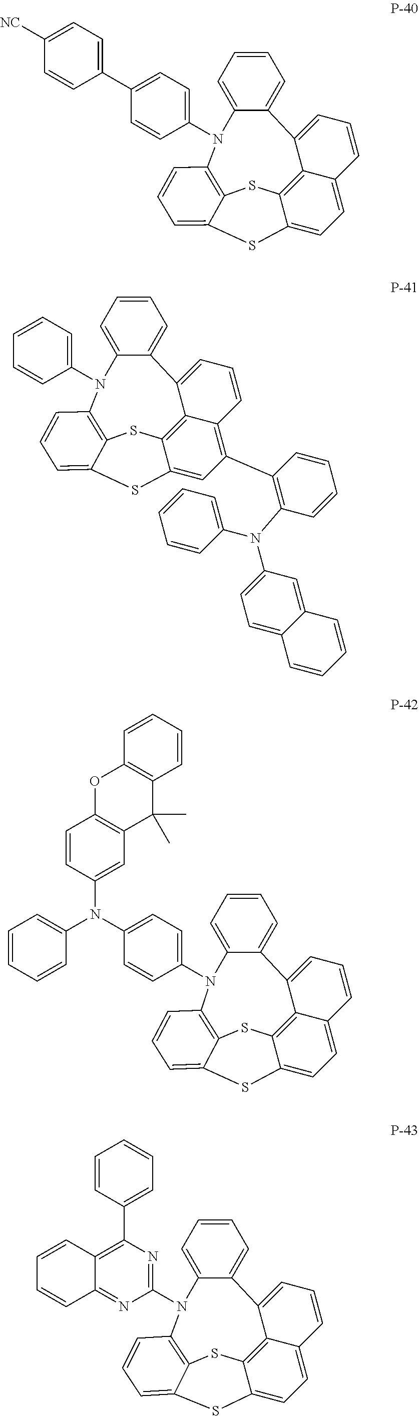 [SMILES: N#Cc1ccc(-c2ccc(N3c4ccccc4-c4cccc5ccc6c(c45)Sc4c(cccc43)S6)cc2)cc1 ; c1ccc(N(c2ccc3ccccc3c2)c2ccccc2-c2cc3c4c5c(cccc25)-c2ccccc2N(c2ccccc2)c2cccc(c2S4)S3)cc1 ; CC1(C)c2ccccc2Oc2ccc(N(c3ccccc3)c3ccc(N4c5ccccc5-c5cccc6ccc7c(c56)Sc5c(cccc54)S7)cc3)cc21 ; c1ccc(-c2nc(N3c4ccccc4-c4cccc5ccc6c(c45)Sc4c(cccc43)S6)nc3ccccc23)cc1]

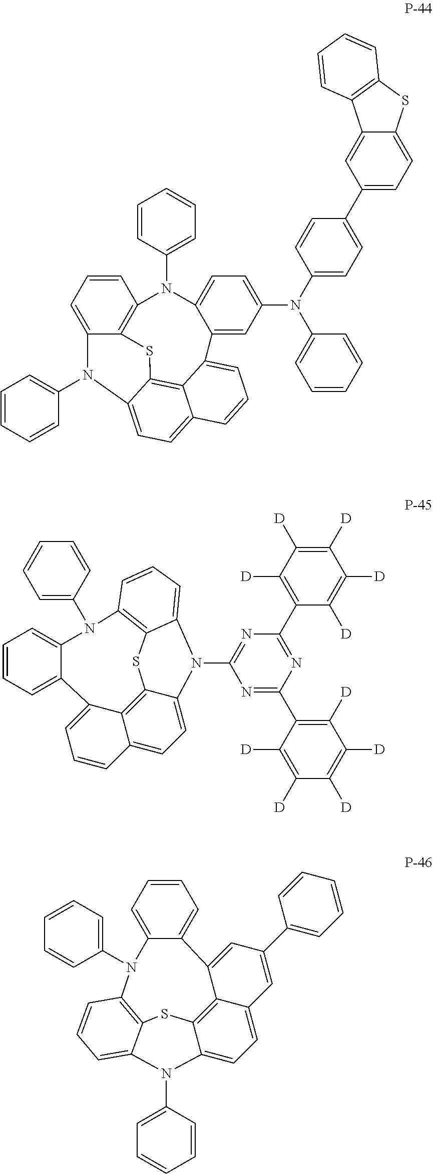 [SMILES: c1ccc(N(c2ccc(-c3ccc4sc5ccccc5c4c3)cc2)c2ccc3c(c2)c2cccc4ccc5c(sc6c(cccc6n5-c5ccccc5)n3-c3ccccc3)c42)cc1 ; [2H]c1c([2H])c([2H])c(-c2nc(-c3c([2H])c([2H])c([2H])c([2H])c3[2H])nc(-n3c4ccc5cccc6c7ccccc7n(-c7ccccc7)c7cccc3c7sc4c56)n2)c([2H])c1[2H] ; c1ccc(-c2cc3ccc4c5sc6c(cccc6n4-c4ccccc4)n(-c4ccccc4)c4ccccc4c(c2)c35)cc1]

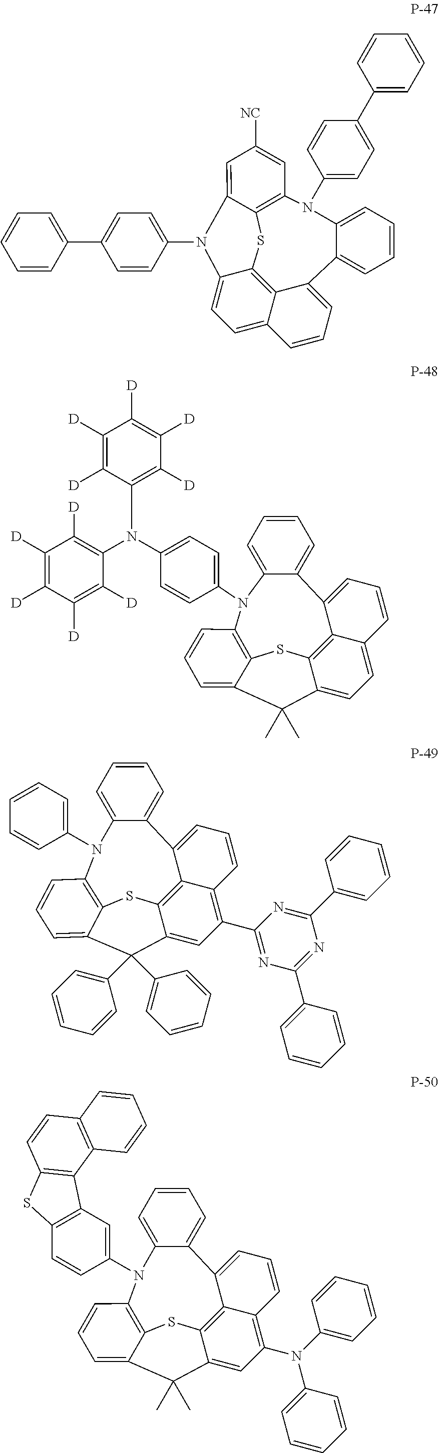 [SMILES: N#Cc1cc2c3sc4c5c(cccc5c5ccccc5n2-c2ccc(-c5ccccc5)cc2)ccc4n(-c2ccc(-c4ccccc4)cc2)c3c1 ; [2H]c1c([2H])c([2H])c(N(c2ccc(N3c4ccccc4-c4cccc5ccc6c(c45)Sc4c3cccc4C6(C)C)cc2)c2c([2H])c([2H])c([2H])c([2H])c2[2H])c([2H])c1[2H] ; c1ccc(-c2nc(-c3ccccc3)nc(-c3cc4c5c6c(cccc36)-c3ccccc3N(c3ccccc3)c3cccc(c3S5)C4(c3ccccc3)c3ccccc3)n2)cc1 ; CC1(C)c2cccc3c2Sc2c1cc(N(c1ccccc1)c1ccccc1)c1cccc(c21)-c1ccccc1N3c1ccc2sc3ccc4ccccc4c3c2c1]

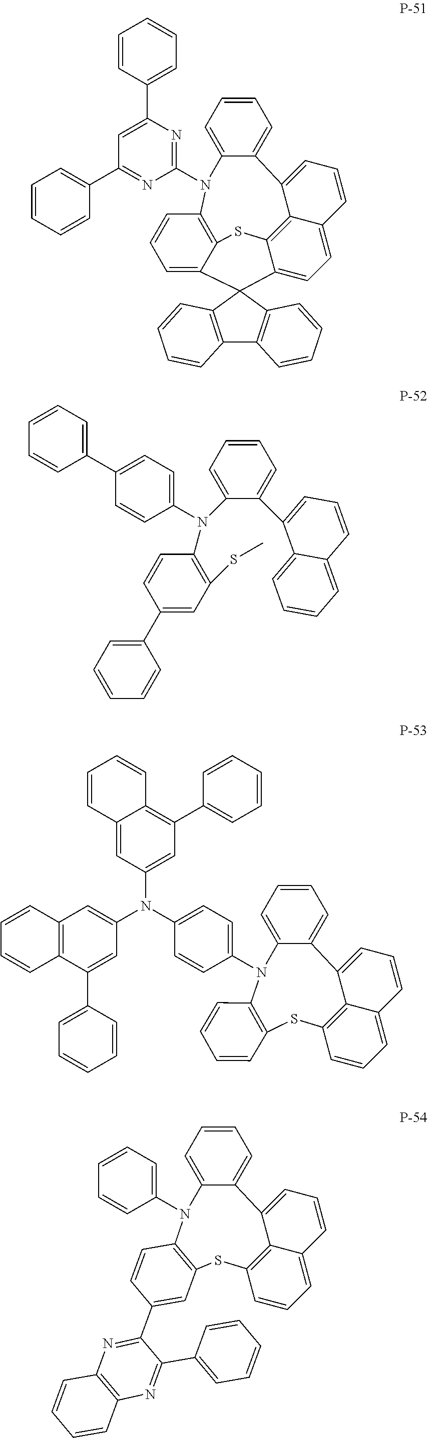 [SMILES: c1ccc(-c2cc(-c3ccccc3)nc(N3c4ccccc4-c4cccc5ccc6c(c45)Sc4c3cccc4C63c4ccccc4-c4ccccc43)n2)cc1 ; CSc1cc(-c2ccccc2)ccc1N(c1ccc(-c2ccccc2)cc1)c1ccccc1-c1cccc2ccccc12 ; c1ccc(-c2cc(N(c3ccc(N4c5ccccc5Sc5cccc6cccc(c56)-c5ccccc54)cc3)c3cc(-c4ccccc4)c4ccccc4c3)cc3ccccc23)cc1 ; c1ccc(-c2nc3ccccc3nc2-c2ccc3c(c2)Sc2cccc4cccc(c24)-c2ccccc2N3c2ccccc2)cc1]

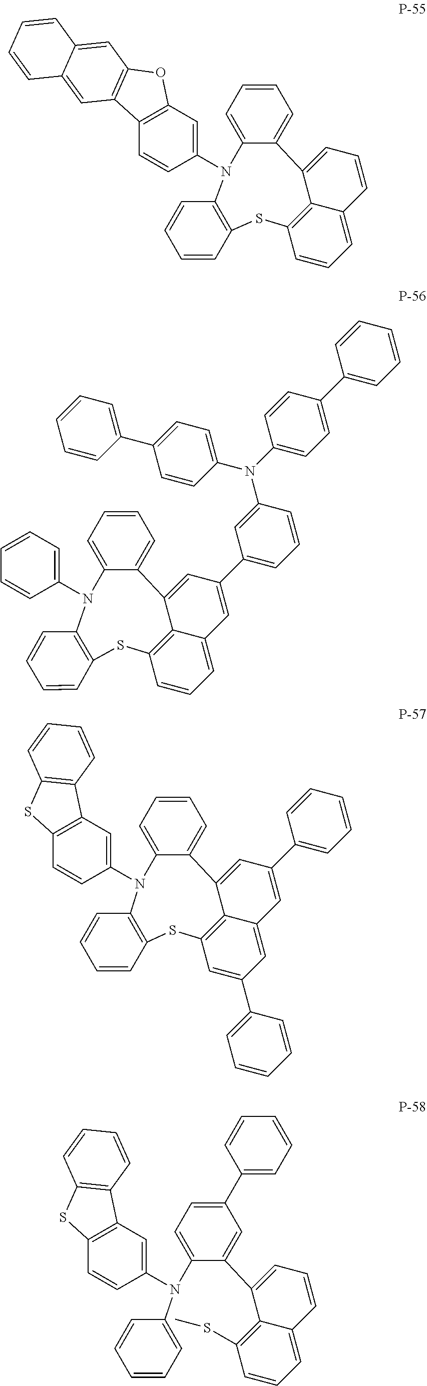

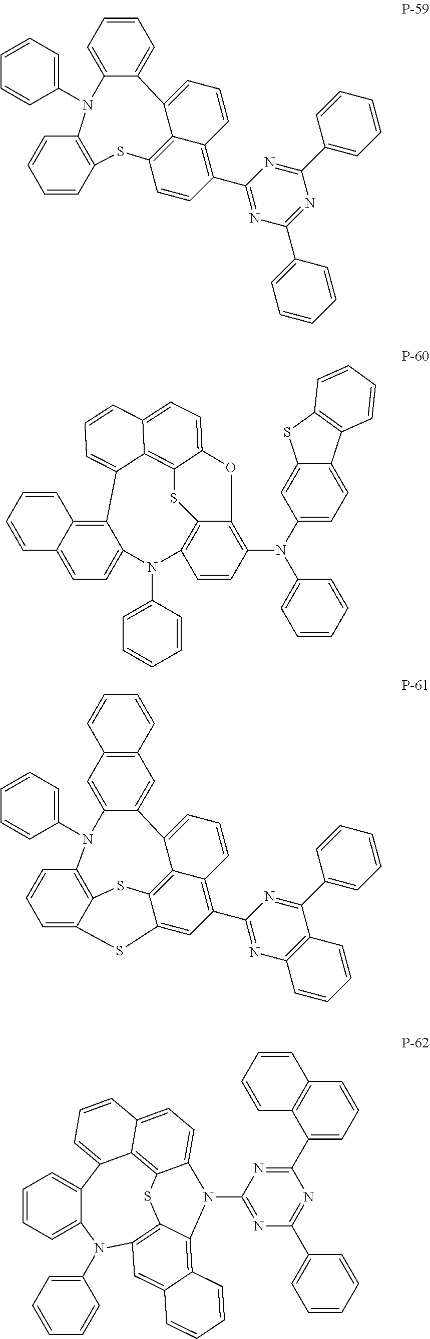

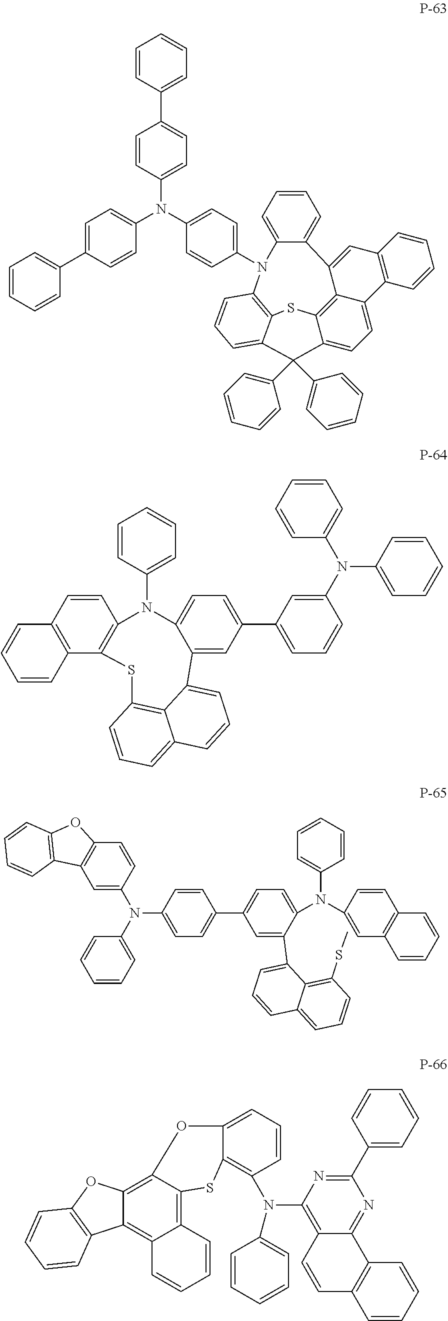 [SMILES: c1ccc(-c2ccc(N(c3ccc(-c4ccccc4)cc3)c3ccc(N4c5ccccc5-c5cc6ccccc6c6ccc7c(c56)Sc5c4cccc5C7(c4ccccc4)c4ccccc4)cc3)cc2)cc1 ; c1ccc(N(c2ccccc2)c2cccc(-c3ccc4c(c3)-c3cccc5cccc(c35)Sc3c(ccc5ccccc35)N4c3ccccc3)c2)cc1 ; CSc1cccc2cccc(-c3cc(-c4ccc(N(c5ccccc5)c5ccc6oc7ccccc7c6c5)cc4)ccc3N(c3ccccc3)c3ccc4ccccc4c3)c12 ; c1ccc(-c2nc(N(c3ccccc3)c3cccc4c3Sc3c(c5oc6ccccc6c5c5ccccc35)O4)c3ccc4ccccc4c3n2)cc1]

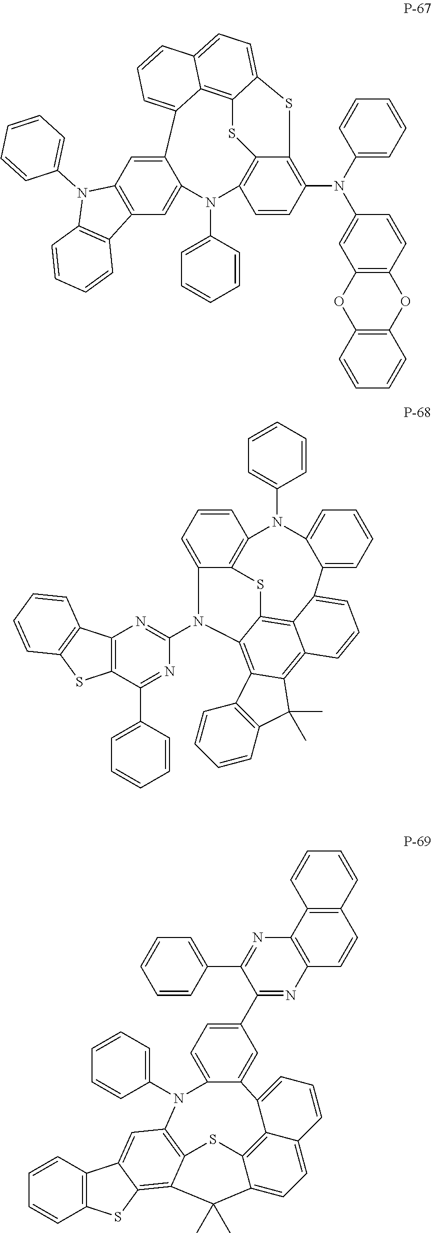 [SMILES: c1ccc(N(c2ccc3c(c2)Oc2ccccc2O3)c2ccc3c4c2Sc2ccc5cccc(c5c2S4)-c2cc4c(cc2N3c2ccccc2)c2ccccc2n4-c2ccccc2)cc1 ; CC1(C)c2ccccc2-c2c1c1cccc3c4ccccc4n(-c4ccccc4)c4cccc5c4sc(c13)c2n5-c1nc(-c2ccccc2)c2sc3ccccc3c2n1 ; CC1(C)c2ccc3cccc4c3c2Sc2c(cc3c(sc5ccccc53)c21)N(c1ccccc1)c1ccc(-c2nc3ccc5ccccc5c3nc2-c2ccccc2)cc1-4]

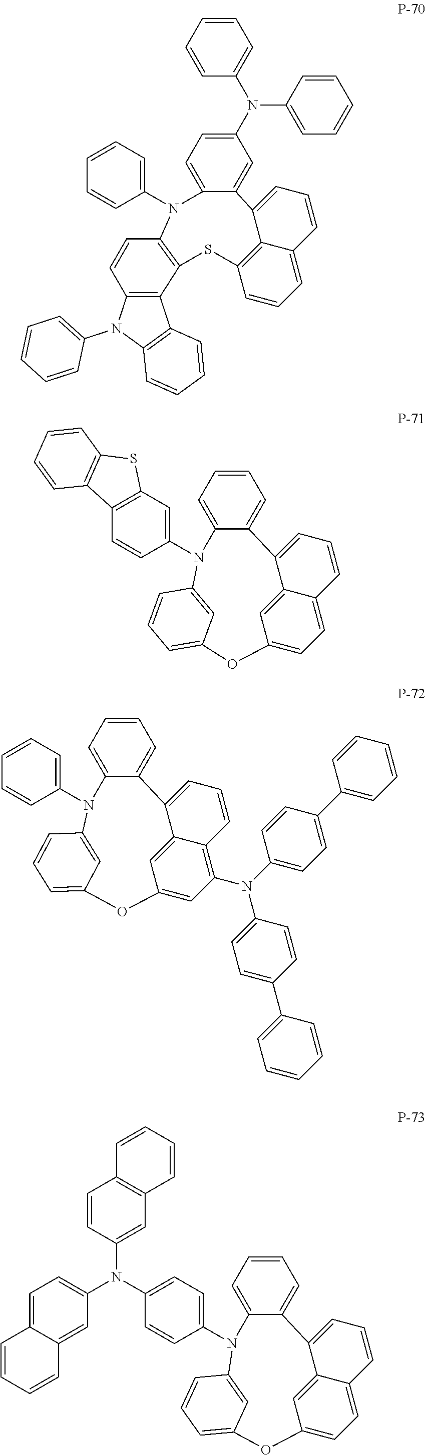 [SMILES: c1ccc(N(c2ccccc2)c2ccc3c(c2)-c2cccc4cccc(c24)Sc2c(ccc4c2c2ccccc2n4-c2ccccc2)N3c2ccccc2)cc1 ; c1cc2cc(c1)N(c1ccc3c(c1)sc1ccccc13)c1ccccc1-c1cccc3ccc(cc13)O2 ; c1ccc(-c2ccc(N(c3ccc(-c4ccccc4)cc3)c3cc4cc5c(cccc35)-c3ccccc3N(c3ccccc3)c3cccc(c3)O4)cc2)cc1 ; c1cc2cc(c1)N(c1ccc(N(c3ccc4ccccc4c3)c3ccc4ccccc4c3)cc1)c1ccccc1-c1cccc3ccc(cc13)O2]

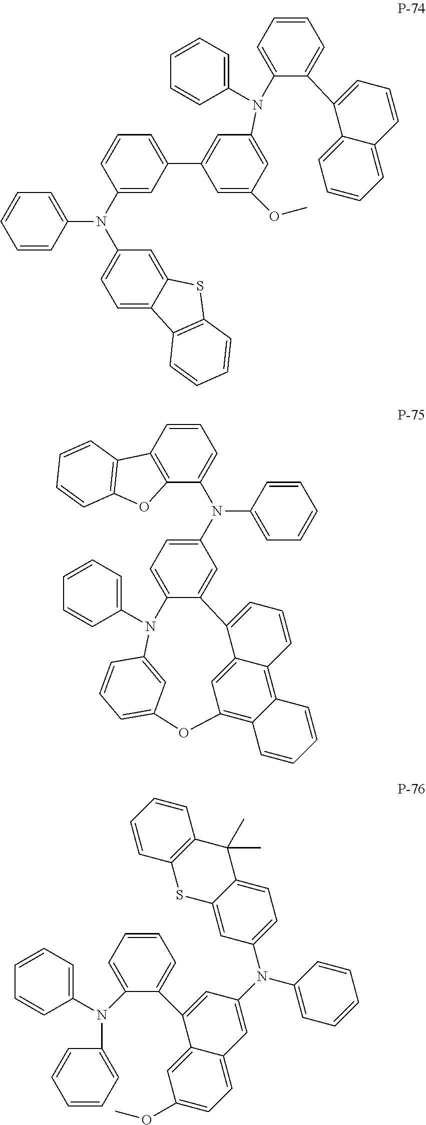

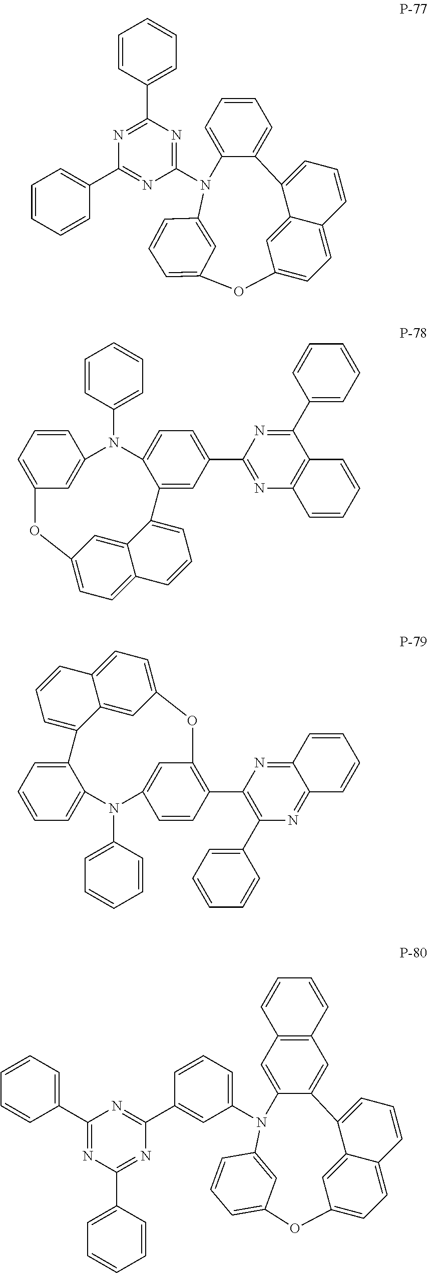 [SMILES: c1ccc(-c2nc(-c3ccccc3)nc(N3c4cccc(c4)Oc4ccc5cccc(c5c4)-c4ccccc43)n2)cc1 ; c1ccc(-c2nc(-c3ccc4c(c3)-c3cccc5ccc(cc35)Oc3cccc(c3)N4c3ccccc3)nc3ccccc23)cc1 ; c1ccc(-c2nc3ccccc3nc2-c2ccc3cc2Oc2ccc4cccc(c4c2)-c2ccccc2N3c2ccccc2)cc1 ; c1ccc(-c2nc(-c3ccccc3)nc(-c3cccc(N4c5cccc(c5)Oc5ccc6cccc(c6c5)-c5cc6ccccc6cc54)c3)n2)cc1]

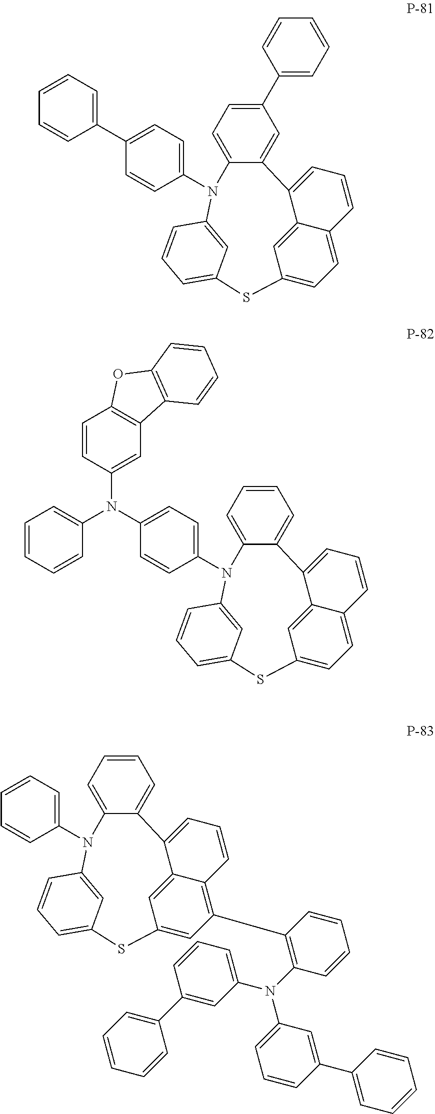 [SMILES: c1ccc(-c2ccc(N3c4cccc(c4)Sc4ccc5cccc(c5c4)-c4cc(-c5ccccc5)ccc43)cc2)cc1 ; c1ccc(N(c2ccc(N3c4cccc(c4)Sc4ccc5cccc(c5c4)-c4ccccc43)cc2)c2ccc3oc4ccccc4c3c2)cc1 ; c1ccc(-c2cccc(N(c3cccc(-c4ccccc4)c3)c3ccccc3-c3cc4cc5c(cccc35)-c3ccccc3N(c3ccccc3)c3cccc(c3)S4)c2)cc1]

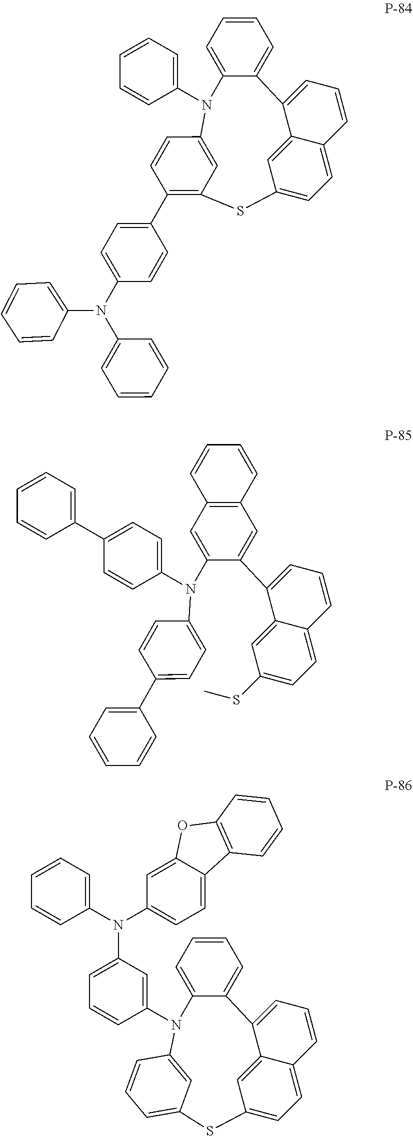 [SMILES: c1ccc(N(c2ccccc2)c2ccc(-c3ccc4cc3Sc3ccc5cccc(c5c3)-c3ccccc3N4c3ccccc3)cc2)cc1 ; CSc1ccc2cccc(-c3cc4ccccc4cc3N(c3ccc(-c4ccccc4)cc3)c3ccc(-c4ccccc4)cc3)c2c1 ; c1ccc(N(c2cccc(N3c4cccc(c4)Sc4ccc5cccc(c5c4)-c4ccccc43)c2)c2ccc3c(c2)oc2ccccc23)cc1]

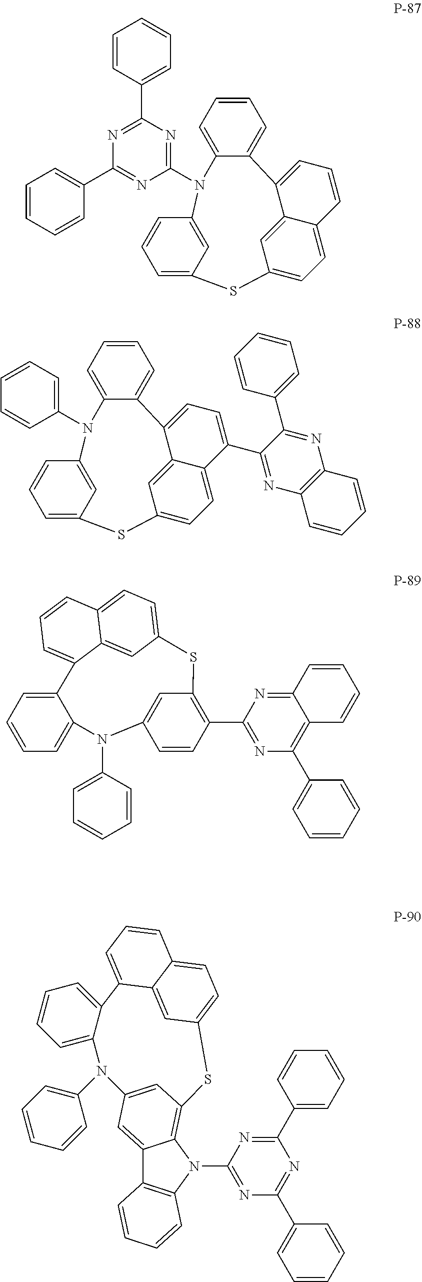 [SMILES: c1ccc(-c2nc(-c3ccccc3)nc(N3c4cccc(c4)Sc4ccc5cccc(c5c4)-c4ccccc43)n2)cc1 ; c1ccc(-c2nc3ccccc3nc2-c2ccc3c4cc(ccc24)Sc2cccc(c2)N(c2ccccc2)c2ccccc2-3)cc1 ; c1ccc(-c2nc(-c3ccc4cc3Sc3ccc5cccc(c5c3)-c3ccccc3N4c3ccccc3)nc3ccccc23)cc1 ; c1ccc(-c2nc(-c3ccccc3)nc(-n3c4ccccc4c4cc5cc(c43)Sc3ccc4cccc(c4c3)-c3ccccc3N5c3ccccc3)n2)cc1]

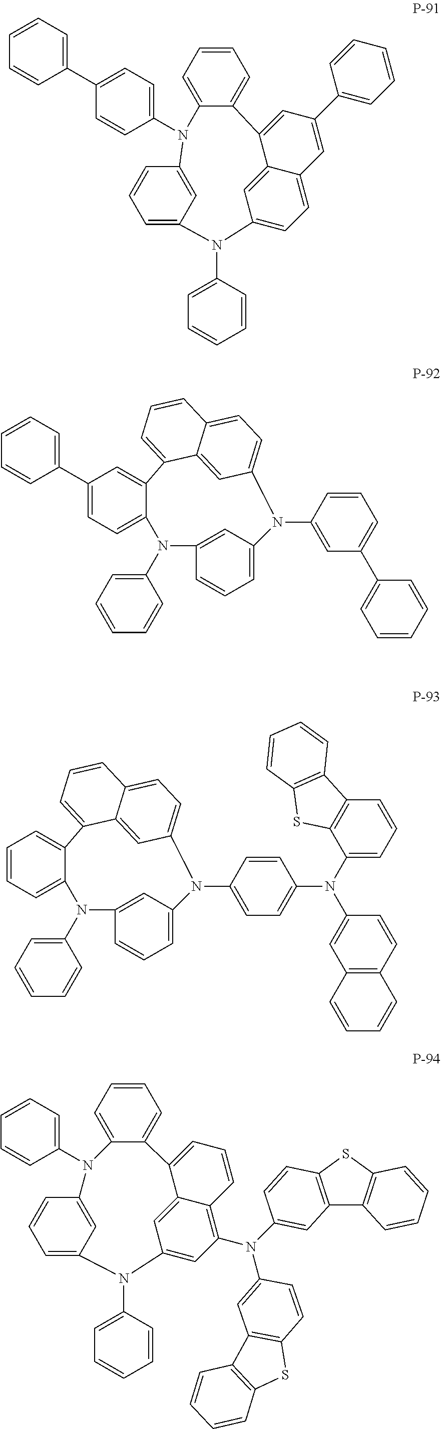 [SMILES: c1ccc(-c2ccc(-n3c4cccc(c4)n(-c4ccccc4)c4ccc5cc(-c6ccccc6)cc(c5c4)c4ccccc43)cc2)cc1 ; c1ccc(-c2cccc(-n3c4ccc5cccc(c5c4)c4cc(-c5ccccc5)ccc4n(-c4ccccc4)c4cccc3c4)c2)cc1 ; c1ccc(-n2c3cccc(c3)n(-c3ccc(N(c4ccc5ccccc5c4)c4cccc5c4sc4ccccc45)cc3)c3ccc4cccc(c4c3)c3ccccc32)cc1 ; c1ccc(-n2c3cc(N(c4ccc5sc6ccccc6c5c4)c4ccc5sc6ccccc6c5c4)c4cccc(c4c3)c3ccccc3n(-c3ccccc3)c3cccc2c3)cc1]

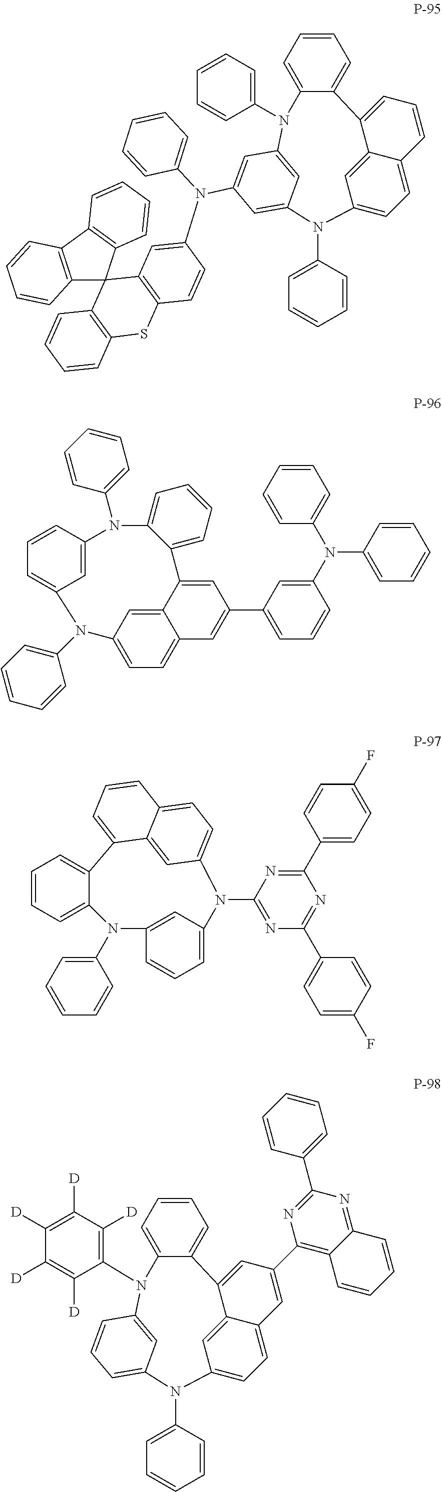 [SMILES: c1ccc(N(c2ccc3c(c2)C2(c4ccccc4S3)c3ccccc3-c3ccccc32)c2cc3cc(c2)n(-c2ccccc2)c2ccccc2c2cccc4ccc(cc42)n3-c2ccccc2)cc1 ; c1ccc(N(c2ccccc2)c2cccc(-c3cc4ccc5cc4c(c3)c3ccccc3n(-c3ccccc3)c3cccc(c3)n5-c3ccccc3)c2)cc1 ; Fc1ccc(-c2nc(-c3ccc(F)cc3)nc(-n3c4ccc5cccc(c5c4)c4ccccc4n(-c4ccccc4)c4cccc3c4)n2)cc1 ; [2H]c1c([2H])c([2H])c(-n2c3cccc(c3)n(-c3ccccc3)c3ccc4cc(-c5nc(-c6ccccc6)nc6ccccc56)cc(c4c3)c3ccccc32)c([2H])c1[2H]]

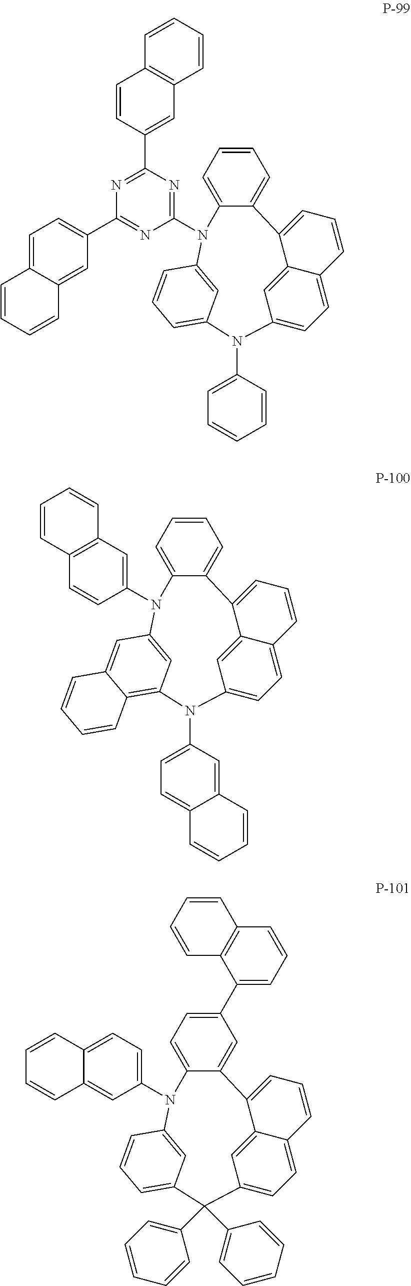

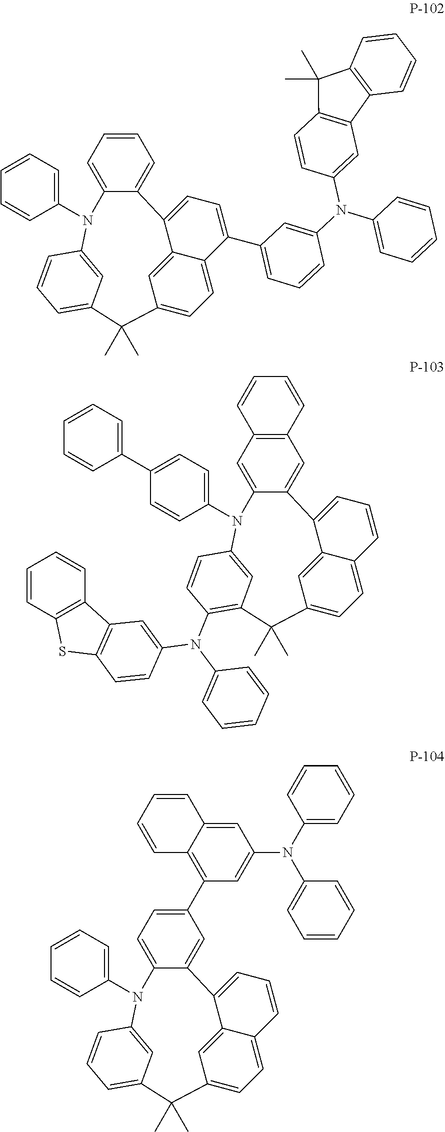 [SMILES: CC1(C)c2cccc(c2)N(c2ccccc2)c2ccccc2-c2ccc(-c3cccc(N(c4ccccc4)c4ccc5c(c4)-c4ccccc4C5(C)C)c3)c3ccc1cc23 ; CC1(C)c2ccc3cccc(c3c2)-c2cc3ccccc3cc2N(c2ccc(-c3ccccc3)cc2)c2ccc(N(c3ccccc3)c3ccc4sc5ccccc5c4c3)c1c2 ; CC1(C)c2cccc(c2)N(c2ccccc2)c2ccc(-c3cc(N(c4ccccc4)c4ccccc4)cc4ccccc34)cc2-c2cccc3ccc1cc23]

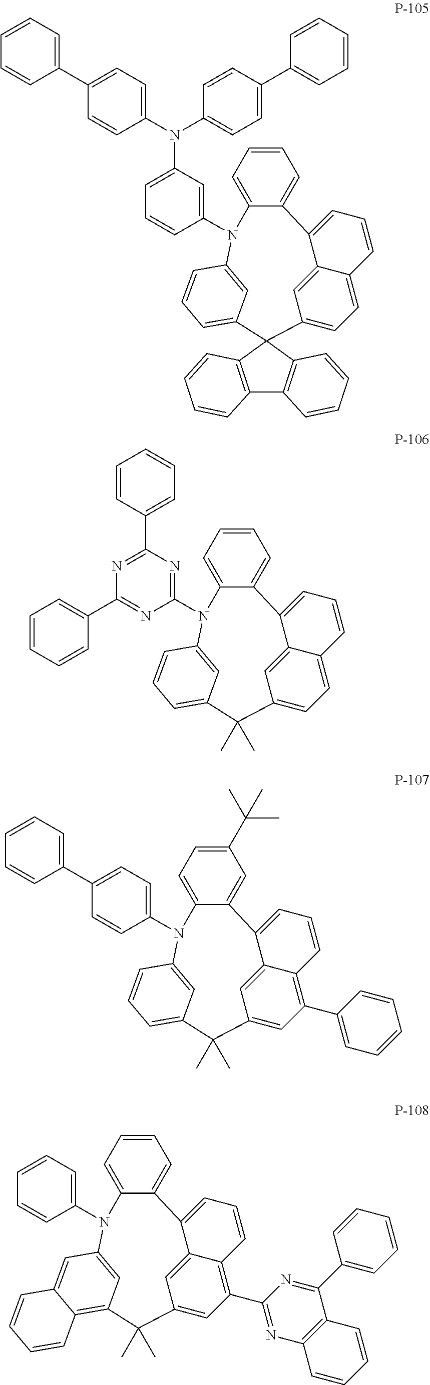

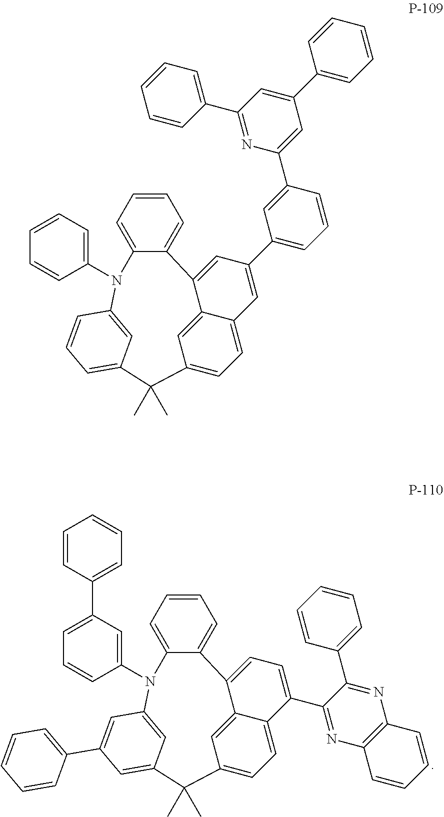 [SMILES: CC1(C)c2cccc(c2)N(c2ccccc2)c2ccccc2-c2cc(-c3cccc(-c4cc(-c5ccccc5)cc(-c5ccccc5)n4)c3)cc3ccc1cc23 ; CC1(C)c2cc(-c3ccccc3)cc(c2)N(c2cccc(-c3ccccc3)c2)c2ccccc2-c2ccc(-c3nc4ccccc4nc3-c3ccccc3)c3ccc1cc23]

7. An organic electronic element comprising an anode, a cathode, and an organic material layer formed between the anode and the cathode, wherein the organic material layer comprises a single compound or 2 or more compounds represented by Formula 1 of claim 1.

8. The organic electronic element of claim 7, wherein the organic material layer comprises at least one of a hole injection layer, a hole transport layer, an emitting-auxiliary layer, an emitting layer, an electron transport auxiliary layer, an electron transport layer, and an electron injection layer.

9. The organic electronic element of claim 7, wherein the organic material layer is an emitting-auxiliary layer.

10. The organic electronic element of claim 7, wherein the organic material layer is an emitting layer.

11. The organic electronic element of claim 7, further comprising a light efficiency enhancing layer formed on at least one surface opposite to the organic material layer among the surfaces of the anode and the cathode.

12. The organic electronic element of claim 7, wherein the organic material layer is a light efficiency improving layer.

13. The organic electronic element of claim 7, wherein the organic material layer comprises 2 or more stacks including a hole transport layer, an emitting layer, and an electron transport layer sequentially formed on the anode.

14. The organic electronic element of claim 7, wherein the organic material layer further comprises a charge generating layer formed between the 2 or more stacks.

15. An electronic device comprising: a display device including the organic electronic element of claim 7; and a control unit for driving the display device.

16. The organic electronic element of claim 15, wherein the organic electronic element is any one of an organic electroluminescent device (OLED), an organic solar cell, an organic photoreceptor (OPC), an organic transistor (organic TFT), and an element for monochromic or white illumination.
